(19) United States
(12) Patent Application Publication
(10) Pub. No.: US 2025/0360108 A1
(43) Pub. Date: Nov. 27, 2025

(54) CATALYTIC INHIBITOR OF PROTEIN PHOSPHATASE 5 ACTIVATES THE EXTRINSIC APOPTOTIC PATHWAY BY DISRUPTING COMPLEX II

(71) Applicant: The Research Foundation for the State University of New York, Albany, NY (US)

(72) Inventors: Mehdi Mollapour, Syracuse, NY (US); Gennady Bratslavsky, Syracuse, NY (US); John D Chisholm, Syracuse, NY (US); Dimitra Bourdoulia, Syracuse, NY (US); Mark R Woodford, Syracuse, NY (US)

(21) Appl. No.: 18/873,996

(22) PCT Filed: Jun. 12, 2023
(86) PCT No.: PCT/US23/25049

§ 371 Date: Dec. 11, 2024

Related U.S. Application Data
(60) Provisional application No. 63/351,433, filed on Jun. 12, 2022.

(51) Int. Cl. A61K 31/42; A61K 31/18; A61K 31/60; A61K 31/606; A61K 31/609; A61K 31/625

(52) U.S. Cl. CPC A61K 31/42; A61K 31/18; A61K 31/60; A61K 31/606; A61K 31/609; A61K 31/625

(57) ABSTRACT

Protein phosphatase 5 (PP5) is a serine/threonine protein phosphatase involved in the maturation and activation of numerous signaling pathways essential for cancer growth. PP5 activity is essential for the survival of clear cell renal cell carcinoma (ccRCC), however the mechanism remains unclear. Data demonstrates that PP5 interacts with caspase-8, FADD, and RIPK1, components of extrinsic apoptotic pathway Complex II. Specifically, PP5 dephosphorylates and inactivates the death effector proteins RIPK1 and FADD, preserving Complex II integrity and regulating extrinsic apoptosis. Protein phosphatases are considered to be ‘undruggable,’ however we have developed a specific inhibitor of PP5 (P-053) that prevents substrate binding to the active site. Encouragingly, PP5 inhibition using P-53 in VHL-null ccRCC robustly induces extrinsic apoptosis. Taken together, the data suggests that PP5 promotes ccRCC survival by suppressing extrinsic apoptosis, and small molecule inhibition of PP5 presents a viable therapeutic strategy for ccRCC.

### FIELD OF THE INVENTION

The present disclosure is in the fields of organic chemistry, medicine, and cancer therapy. More particularly, it concerns therapeutic and preventative agents, such as protein phosphatase-5 (PP5) inhibitors for treating or preventing diseases, disorders and conditions involving cancer. For example, protein phosphatase-5 (PP5) promotes clear cell renal cell carcinoma (ccRCC) survival by suppressing extrinsic apoptosis. Small molecule specific inhibition of PP5 presents a viable therapeutic strategy for cancer such as ccRCC.

### BACKGROUND

The serine/threonine protein phosphatase PP5 regulates several signaling-cascades that are responsible for tumor initiation, progression and metastasis (Golden et al., 2008) (Dushukyan et al., 2017). A single gene encodes PP5 and its regulatory and catalytic domains are all contained within the same polypeptide (Oberoi, et al., 2016, Shi, 2009). PP5 generally has low basal activity because the tetratricopeptide repeat (TPR) motif at its amino-terminus interacts with the αJ-helix in the carboxy-terminus. This prevents substrates from entering the active site of PP5 (Cliff, et al., 2006, Haslbeck, et al., 2015, Kang, et al., 2001, Ramsey and Chinkers, 2002, Yang, et al., 2005). PP5 is a co-chaperone of the molecular chaperone Heat shock protein-90 (Hsp90). Activation of PP5 depends on binding of its TPR-domain to the molecular chaperone Hsp90 and client substrates. Other cellular factors such as polyunsaturated fatty acids also activate PP5 (Chatterjee, et al., 2010, Ramsey and Chinkers, 2002, Yang, et al., 2005). Post-translational modifications of PP5 play a major role in its switching “on” and “off” in cells.

However, even though PP5 regulates several signaling cascades responsible for tumor initiation, progression and metastasis, protein phosphatases are considered to be ‘undruggable,’ and active pharmaceutical ingredients or drugs relating to these compounds are not specific resulting in detrimental side effects.

There is a continuous need for PP5 inhibitors that possess the appropriate pharmacological properties to be safe and therapeutically useful while providing effective inhibition. Such compounds would be extremely useful in treating the disease states where inhibition could play a role.

### SUMMARY

The present disclosure now provides PP5 inhibitors including the appropriate pharmacological properties that are safe and therapeutically useful while providing effective inhibition. Such compounds are excellent for treating the disease states where inhibition could play a role. Moreover, the present disclosure provides one or more specific inhibitors of PP5 that, among other things, prevent substrate binding to the active site of PP5. Further, the present disclosure now provides for PP5 inhibition using pharmaceutically acceptable PP5 inhibitors to robustly induce extrinsic apoptosis in cancer cells.

In embodiments, the present disclosure provides a method of treating cancer, including: a) providing i) a subject with cancer, ii) one or more protein phosphatase-5 inhibitors, and b) treating said subject with the one or more PP5 inhibitors of the present disclosure. In embodiments, treating includes administering a therapeutically effective amount of one or more protein phosphatase-5 inhibitors to the subject in need thereof.

In embodiments, the present disclosure includes a method of treating cancer, including: a) providing i) a subject with cancer and one or more compounds of:

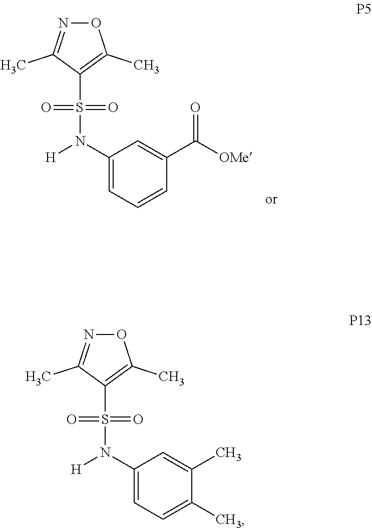

and
b) treating the subject with the one or more compounds or pharmaceutically acceptable salts thereof.

In embodiments, the present disclosure includes a method of treating cancer, including: a) providing i) a subject with cancer and one or more compounds of:

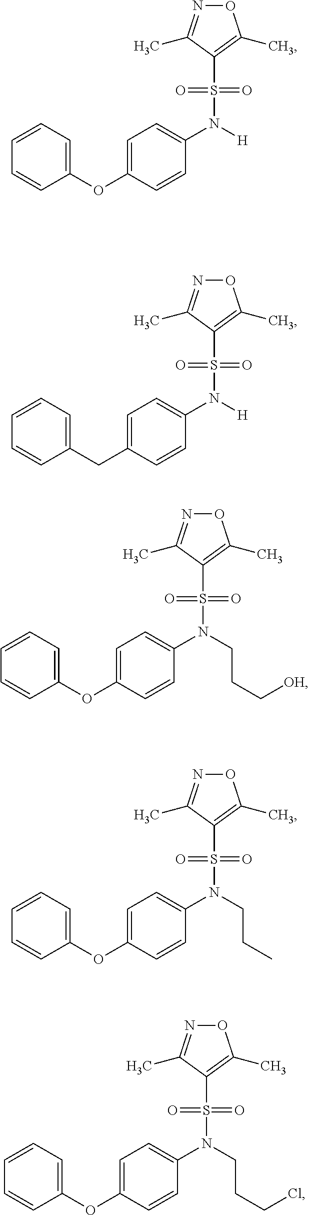 [SMILES: Cc1noc(C)c1S(=O)(=O)Nc1ccc(Oc2ccccc2)cc1 ; Cc1noc(C)c1S(=O)(=O)Nc1ccc(Cc2ccccc2)cc1 ; Cc1noc(C)c1S(=O)(=O)N(CCCO)c1ccc(Oc2ccccc2)cc1 ; CCCN(c1ccc(Oc2ccccc2)cc1)S(=O)(=O)c1c(C)noc1C ; Cc1noc(C)c1S(=O)(=O)N(CCCCl)c1ccc(Oc2ccccc2)cc1]

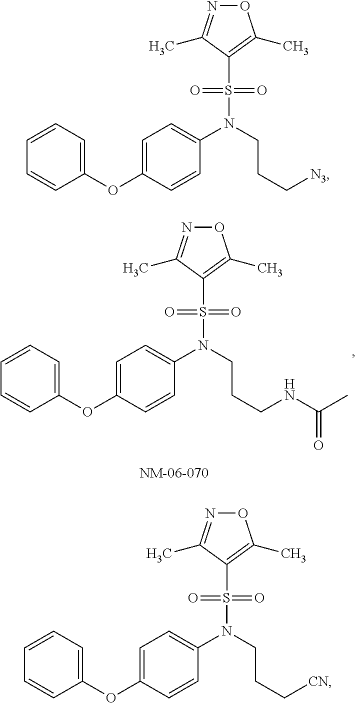

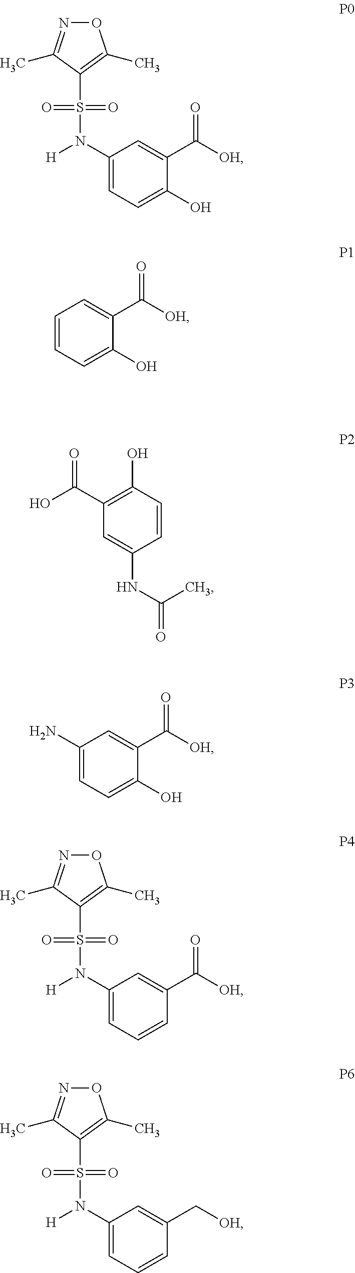 [SMILES: Cc1noc(C)c1S(=O)(=O)Nc1ccc(O)c(C(=O)O)c1 ; O=C(O)c1ccccc1O ; CC(=O)Nc1ccc(O)c(C(=O)O)c1 ; Nc1ccc(O)c(C(=O)O)c1 ; Cc1noc(C)c1S(=O)(=O)Nc1cccc(C(=O)O)c1 ; Cc1noc(C)c1S(=O)(=O)Nc1cccc(CO)c1]

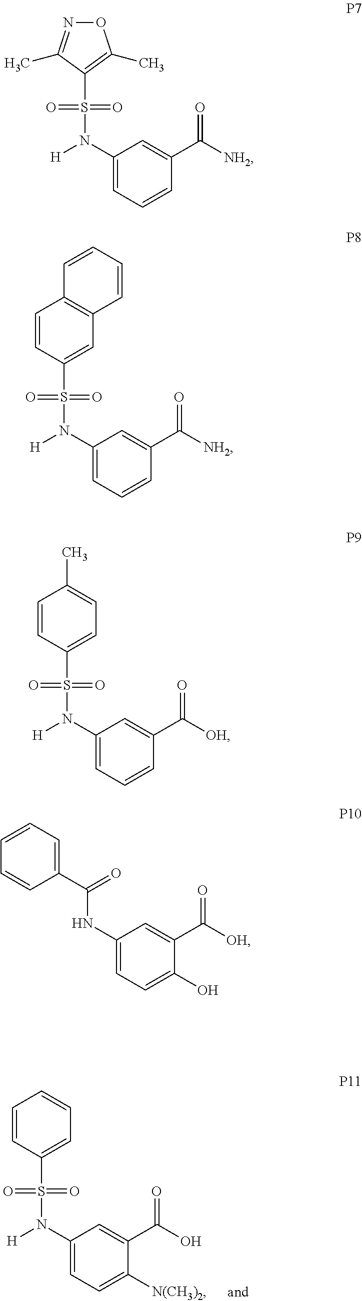 [SMILES: Cc1noc(C)c1S(=O)(=O)Nc1cccc(C(N)=O)c1 ; NC(=O)c1cccc(NS(=O)(=O)c2ccc3ccccc3c2)c1 ; Cc1ccc(S(=O)(=O)Nc2cccc(C(=O)O)c2)cc1 ; O=C(Nc1ccc(O)c(C(=O)O)c1)c1ccccc1 ; CN(C)c1ccc(NS(=O)(=O)c2ccccc2)cc1C(=O)O]

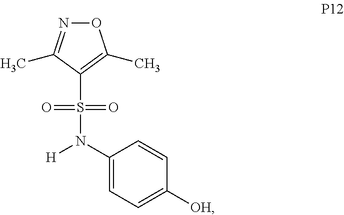 [SMILES: Cc1noc(C)c1S(=O)(=O)Nc1ccc(O)cc1]

and b) treating the subject with the one or more compounds. In embodiments, the one or more compounds are administered to a subject in need thereof in a pharmaceutically effective amount.

In embodiments, the present disclosure includes a pharmaceutical anticancer composition including one or more PP5 inhibitors of the present disclosure, or pharmaceutically acceptable salts thereof.

In embodiments, the present disclosure includes a method of destabilizing complex IIA and inducing an extrinsic apoptotic pathway, including, contacting one or more cancer cells with one or more PP5 inhibitors of the present disclosure.

The described features, structures, or characteristics of the invention may be combined in any suitable manner in one or more embodiments. In the following description, numerous specific details are recited to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

The illustrative aspects of the present disclosure are designed to solve the problems herein described and/or other problems not discussed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1H present the identification and characterization of small molecule inhibitors of PP5. FIG. 1A presents a schematic workflow of in silico screening a library of ˜3.7-million compounds to identify potential PP5 inhibitors. A single overlapping hit, P0 was used to identify a set of analogs for cell-based screening. Compounds 5 (P5) and 13 (P13) were selected as candidate PP5 inhibitors. FIG. 1B presents 786-O cells were treated with the indicated amounts of P5 and P13 for 48 hrs. The effect of PP5 inhibitors on cell viability was assessed by MTT assay. Error bars represent the standard deviation (S.D.) of three independent experiments. A Student's t-test was performed to assess statistical significance **P<0.01; ***P<0.001. FIG. 1C presents 786-O cells were treated with 10 μM P5 or P13 or DMSO vehicle control for 24 hrs. Induction of apoptosis was assessed by immunoblotting. GAPDH was used as a loading control. FIG. 1D presents a PP5 enzyme kinetic data with P5 (orange line), P13 (red line) or no inhibitor (blue line) presented as a Lineweaver-Burk plot (n=3 independent samples). FIG. 1E presents Lysates from HEK293 cells transfected with PP5-FLAG were incubated with indicated amounts of biotin-labeled P5 and P13 followed by streptavidin agarose pulldown. Co-pulldown of PP5-FLAG was detected by immunoblotting. FIG. 1F presents crystal structure of PP5 active site (gray; PDB:3H60) with predicted residues responsible (green) for contact with P13 (orange) modeled with PyMOL software (v4.6.0). FIG. 1G presents T-PP5-FLAG and predicted binding residue mutants were transiently expressed in HEK293 cells. Lysate was incubated with indicated amounts of biotinylated P13 followed by streptavidin pulldown (above). PP5 binding to biotinylated P13 was examined by immunoblot. Input expression of PP5-FLAG and mutants is below. EV was used as a control. FIG. 1H presents P13-BODIPY binding to PP5 measured by fluorescence anisotropy. Points are replicate means±SEM from independent trials for display. (n=2).

FIGS. 2A-2H present inhibitory effect of P053 compound on PP5 and induction apoptosis in VHL-null ccRCC. FIG. 2A presents 786-O cells were treated with 10 μM of the indicated second generation of PP5 inhibitors for 24 hrs. DMSO was used as control. PP5 inhibition was evaluated by immunoblotting for the bono fide substrate phospho-S211-GR. SE (short exposure) and LE (long exposure) of the radiographic film. GAPDH was used as a loading control. FIG. 2B presents 786-O cells were again treated with 10 μM of the indicated second generation of PP5 inhibitors for 24 hrs. DMSO was used as control. Induction of apoptosis was evaluated by immunoblotting using apoptotic markers cleaved PARP and cleaved caspase 8. GAPDH was used as a loading control. GIG 2C presents 786-O cells were treated with indicated amounts of P13, P053, P058, P059, and P075 for 24 hrs and then cell proliferation was assessed by the MTT assay. A Student's t-test was performed to assess statistical significance *P<0.05; **P<0.01 or non-significant (ns). FIG. 2D presents PP5 enzyme kinetic data with P053 (purple) or no inhibitor (blue line) presented as a Lineweaver-Burk plot (n=3 independent samples). FIG. 2E presents the structure of PP5 catalytic domain (gray; PDB 3H60) bound to PP5 inhibitors P5 (green), P13 (orange), and P053 (yellow). FIG. 2F presents lysate from 786-O cells was collected and incubated with 1 μM biotinylated P5 or P13 for 1 hr and then challenged with 1 μM P053 for 30 min. Streptavidin-coated agarose beads were used to pull down the biotinylated compounds. Co-pulldown of endogenous PP5 and PP2A was assessed by immunoblotting. SE (short exposure) and LE (long exposure) of the radiographic film. FIG. 2G presents VHL containing HK2, Caki-2, and Caki-1 cells and VHL-null cells, A498 and 786-O, were treated with the indicated amount of P053 for 24 hrs, and then cell proliferation was assessed by the MTT assay. A Student's t-test was performed to assess statistical significance *P<0.05; **P<0.01 or non-significant (ns). FIG. 2H presents Caki-1, Caki-2, A498, and 786-O cells were treated with 30 or 40 μM P053 or DMSO control (0 μM) for 24 hrs. Following propidium iodide (PI) staining cell death was assessed by flow cytometry. Percentage of PI stained cells was normalized to the vehicle control for each cell line individually.

FIGS. 3A-3H present targeting PP5 activates the extrinsic apoptotic pathway. FIG. 3A presents ccRCC 786-O cells were treated with 10 μM compound P053 for 24 hrs. Induction of apoptosis was evaluated by immunoblotting using apoptotic markers as indicated. GAPDH was used as a loading control. FIG. 3B presents PP5 was silenced by small interfering RNA (siRNA) in VHL-null ccRCC cells 786-O and A498. Induction of apoptosis was evaluated by immunoblotting using apoptotic markers as indicated. siCtrl represents the non-targeting siRNA control. GAPDH was used as a loading control. FIG. 3C presents Inhibition of CK1δ by indicated amounts of IC261 for 16 hrs in 786-O cells. Induction of apoptotic markers was assessed by immunoblotting. GAPDH was used as a loading control. FIG. 3D presents ccRCC 786-O cells were treated in presence (+) or absence (−) of 10 μM apoptotic inhibitor z-VAD-fmk for 1 hr followed by the addition of indicated amounts of CK1δ inhibitor, IC261, for an additional 16 hrs. Induction of apoptosis was evaluated by the immunoblotting. GAPDH was used as a loading control. FIG. 3E presents a schematic representation of the extrinsic apoptotic pathway. Death receptors are activated by binding of death ligands. The leads to binding of adaptors and ultimately formation of complex II containing FADD, RIPK1, and pro-caspase 8. Upon complex II formation, caspase 8 is activated and then released from the complex leading to downstream caspase induction and apoptosis. FIG. 3F presents an endogenous PP5 was immunoprecipitated (IP) from 786-O cells. Coimmunoprecipitation (co-IP) of FADD, RIPK1, and caspase 8 was examined by immunoblot. IgG was used as a control. GAPDH was used as a loading control. FIG. 3G presents WT-PP5-FLAG was transiently expressed and isolated from 786-O cells. Co-IP of FADD and GR was examined by immunoblotting. Phosphorylation level of S194-FADD was assessed by immunoblot. PP5 activity was evaluated by immunoblotting for the bono fide substrate phospho-S211-GR as a control. GAPDH was used as a loading control. FIG. 3H presents CK1δ was inhibited with indicated amounts of IC261 for 24 hrs in ccRCC cells 786-O and A498. Induction of apoptotic markers shown by immunoblotting using anti-cleaved caspase-3 antibody. Phosphorylation of S194-FADD was evaluated by western blot. GAPDH was used as a loading control.

FIGS. 4A-4H present PP5 mediates complex II formation. FIG. 4A presents endogenous FADD (left) and RIPK1 (right) were IP from WT-HAP1 and PP5-KO HAP1 cells. Co-IPs of RIPK1 and FADD were examined by immunoblot. GAPDH was used as a loading control. FIG. 4B presents endogenous FADD (left) and PP5 (right) were IP from WT-HAP1 and RIPK1-KO HAP1 cells. Co-IPs of PP5 and FADD were examined by immunoblot. GAPDH was used as a loading control. FIG. 4C presents endogenous RIPK1 (left) and PP5 (right) were IP from WT-HAP1 and FADD-KO HAP1 cells. Co-IPs of PP5 and RIPK1 were examined by immunoblot. GAPDH was used as a loading control. FIG. 4D presents FADD-FLAG and death domain deleted FADD (FADD-FLAG-ADD) were transiently transfected and IP from HEK293 cells. EV was used as a control. Co-IP of RIPK1 and PP5 was examined by immunoblot. GAPDH was used as a loading control. FIG. 4E presents RIPK1-HA and death domain deleted RIPK1 (RIPK1-HA-ADD) were transiently transfected and isolated from HEK293 cells. EV was used as a control. Co-IP of FADD and PP5 was examined by immunoblot. GAPDH was used as a loading control. FIG. 4F presents HEK293 cells were treated with 2 μM SNX-2112 for the indicated times. FADD and RIPK1 protein levels were examined by immunoblot. Bona fide Hsp90 clients Tsc2, Akt and phos-Ser473-Akt were used as a positive control. GAPDH was used as a loading control. FIG. 4G presents FADD was IP from 786-O cells. Co-IP of PP5 and Hsp90 was evaluated by immunoblot. IgG was used as a negative control. GAPDH was used as a loading control. FIG. 4H presents a schematic illustration of PP5-mediated downregulation of the extrinsic apoptotic pathway. On the left, in the absence of PP5, following activation of the death receptors and complex II formation, caspase 8 is cleaved resulting in activation of apoptosis. On the right, when PP5 associates with complex II and dephosphorylates FADD on S194, the downstream apoptotic pathway is suppressed and leads to ccRCC cell survival.

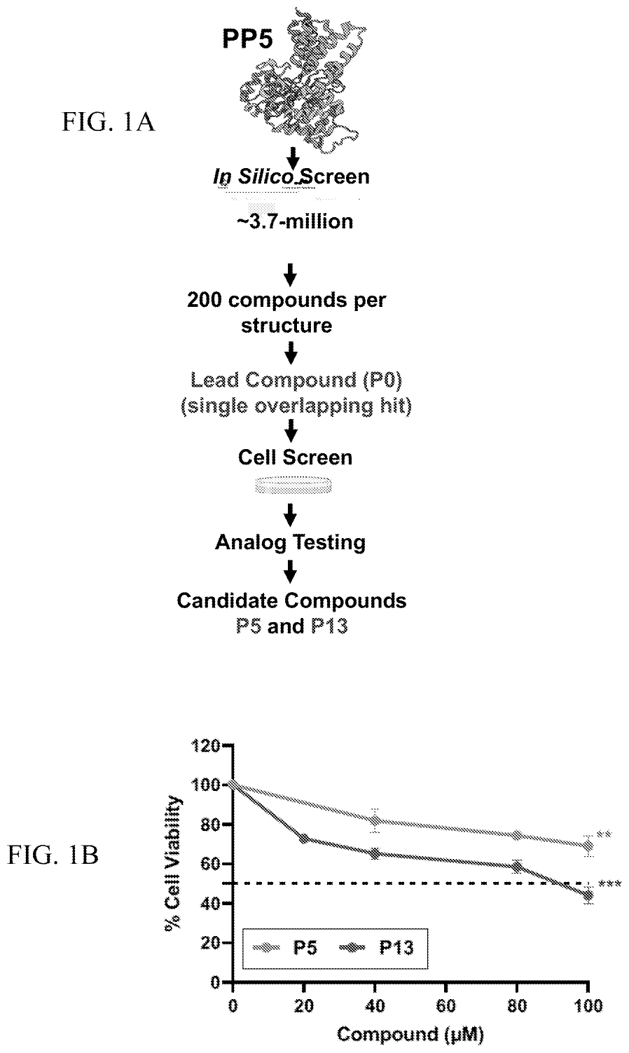

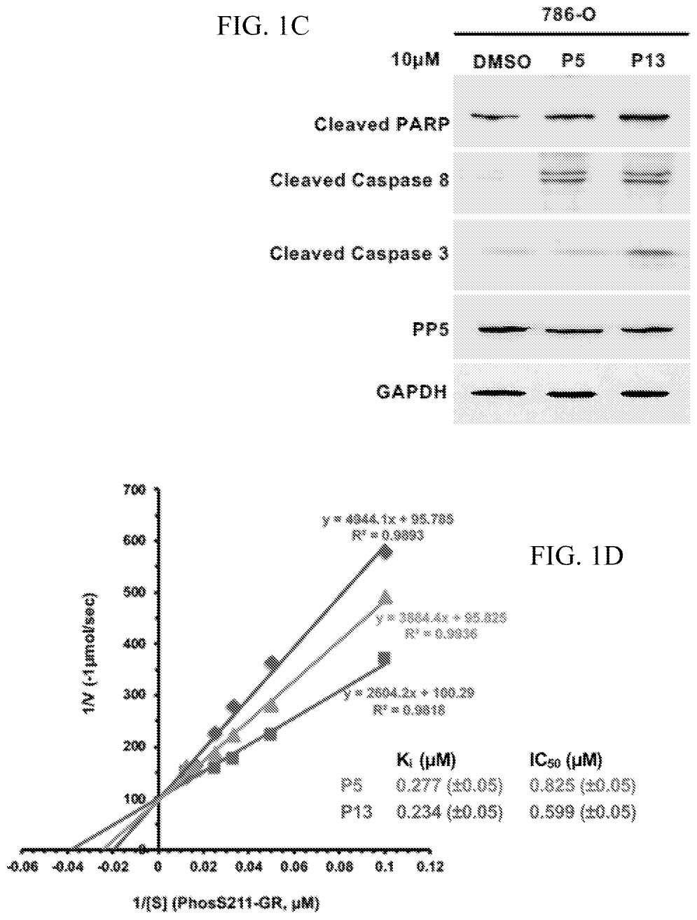

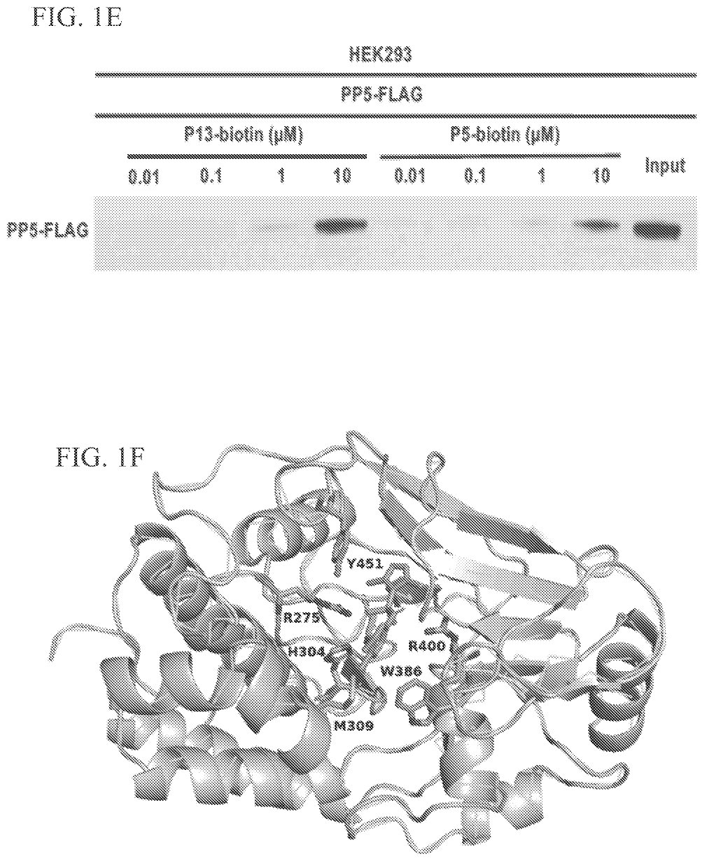

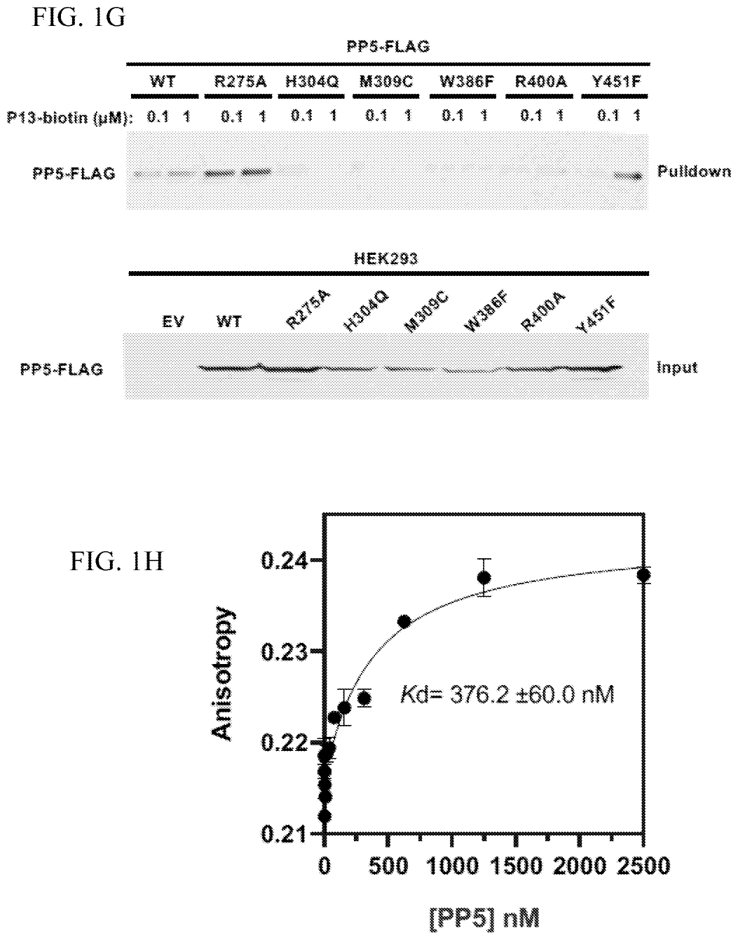

FIGS. S1A-S1F present development of specific inhibitors of PP5 (Related to FIG. 1). FIG. S1A presents In silico docking study used to screen a library of ˜3.7 million compounds using PP5 crystal structure (PDB:3H60). Top-ranked hits were filtered and used for in vitro experiments. Lead compounds are shown bound to PP5 at the final step. FIG. S1B presents P0 inhibitor of PP5 (PDB:3H60) visualized on PyMOL v4.6.0. FIG. S1C presents 786-O cells were treated with indicated amounts of P0 for 24 hrs. DMSO was used as a control. PP5 inhibition was evaluated by immunoblotting for the bono fide substrates phospho-S211-GR and phospho-S13-Cdc37. GAPDH was used as a loading control. FIG. S1D presents chemical structures of 14 identified candidate PP5 inhibitors based on P0 that were used in cell-based screening. FIG. S1E presents 786-O cells were treated with 20 μM P0-P13 for 24 hrs. DMSO was used as a control. Induction of apoptosis was assessed by immunoblotting using cleaved caspase 3. PP5 inhibition was evaluated by immunoblotting for the bono fide substrates phospho-S211-GR and phospho-S13-Cdc37. GAPDH was used as a loading control. FIG. S1F presents 786-O cells were treated with the indicated amounts of selected PP5 inhibitors for 48 hrs. The effect of PP5 inhibitors on cell viability was assessed by MTT assay. Errors bars represent the mean±S.D. of three independent experiments. A Student's t-test was performed to assess statistical significance. *P<0.05; **P<0.01; ***P<0.001; ****P<0.0001.

FIGS. S2A-S2F present chemical synthesis of PP5 inhibitors (Related to FIG. 1). FIG. S2A presents ¹H NMR of P5. FIG. S2B presents ¹³C NMR of P5. FIG. S2C presents ¹H NMR of P13. FIG. S2D presents ¹³C NMR of P13. FIG. S2E presents synthesis of biotin conjugated P5. FIG. S2F presents synthesis of biotin conjugated P13.

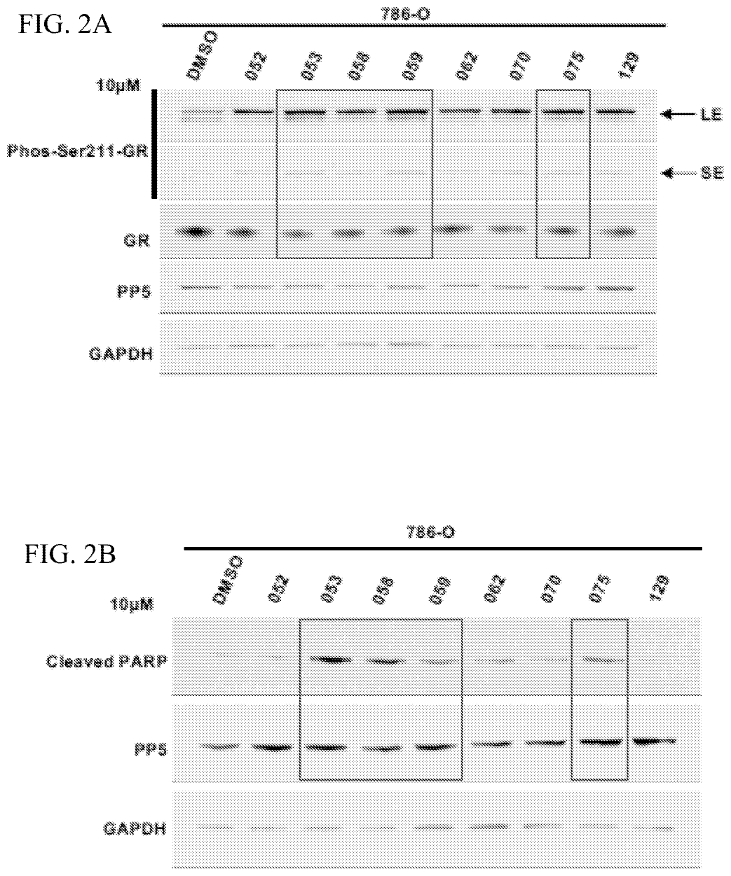

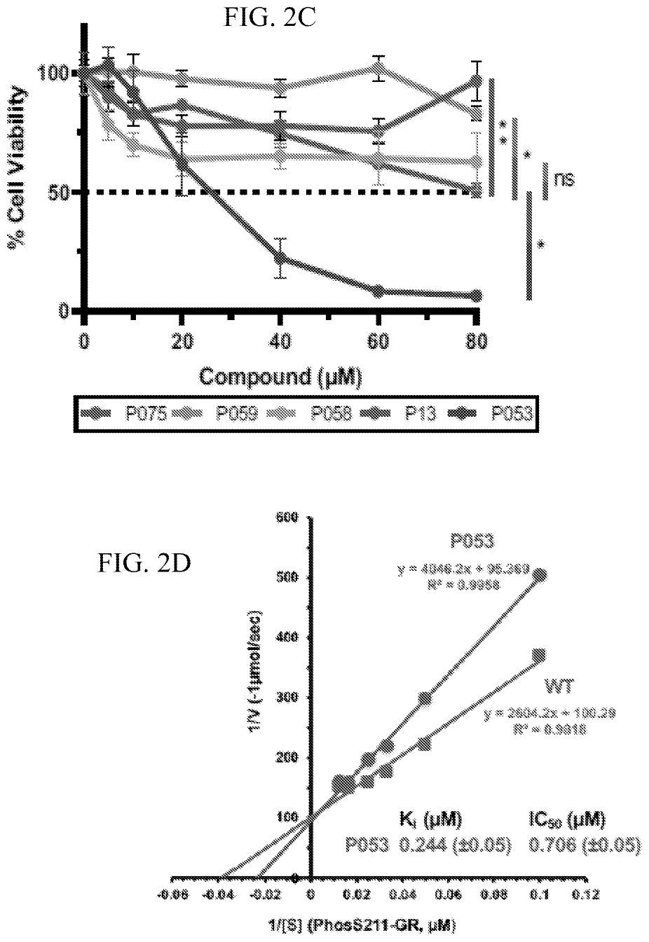

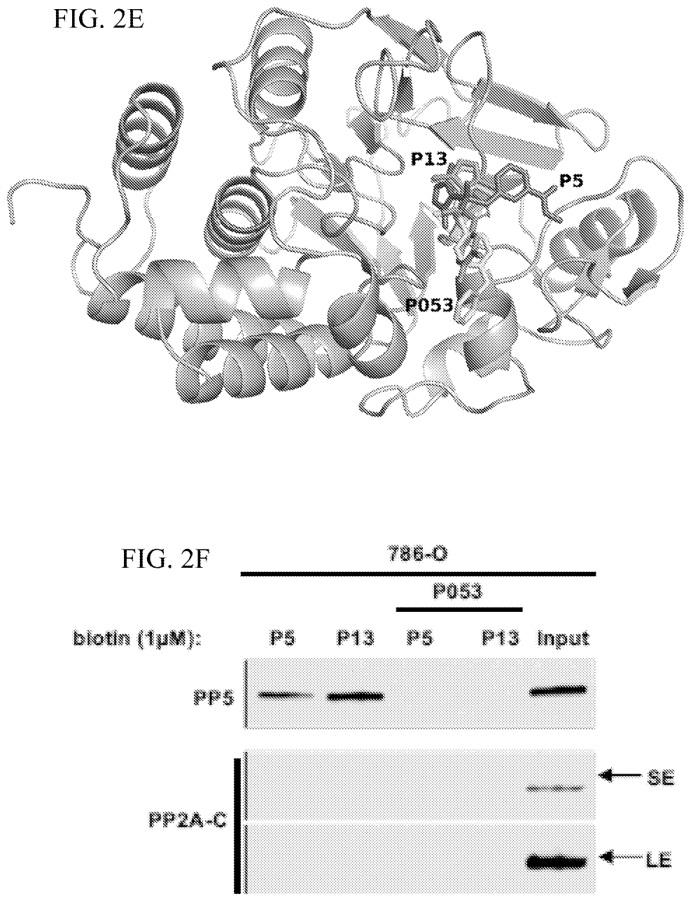

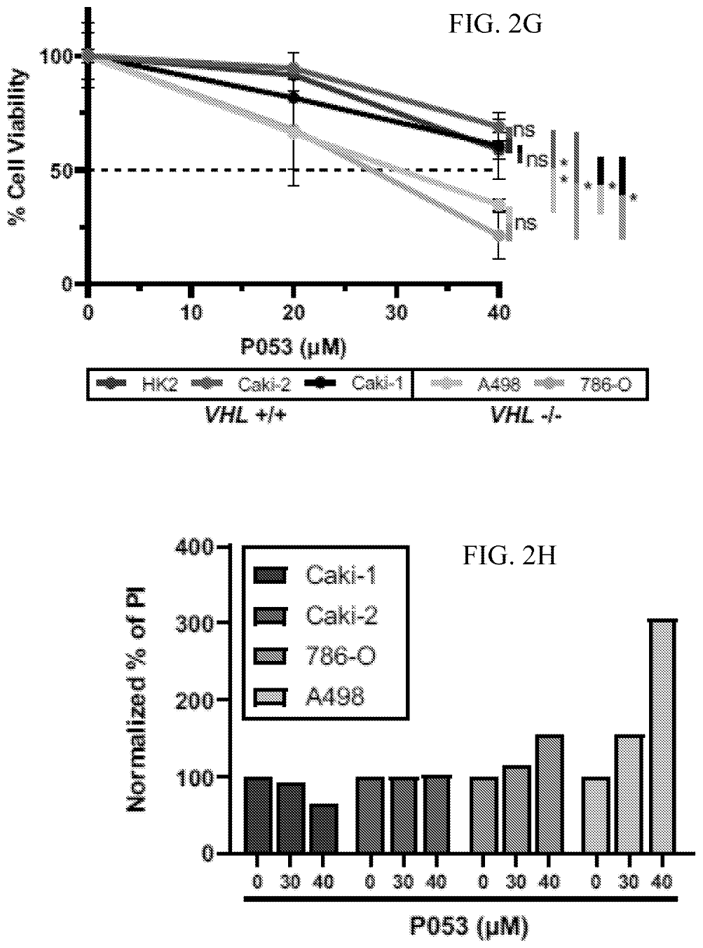

FIGS. S3A-S3F present chemical synthesis of fluorescently labeled P13 second generation of PP5 inhibitors (Related to FIGS. 1-2). FIG. S3A presents synthesis and structure of BODIPY-labeled P13. FIG. S3B presents coomassie-stained SDS PAGE gel showing purified PP5 protein used in FIG. 1H. FIG. S3C presents chemical structures of the second generation PP5 inhibitors derived from P13. FIG. S3D presents synthesis scheme of compounds 16, 17, 18, and 19 from Figure S4A. FIG. S3E presents ¹H NMR of P053. FIG. S3F presents ¹³C NMR of P053.

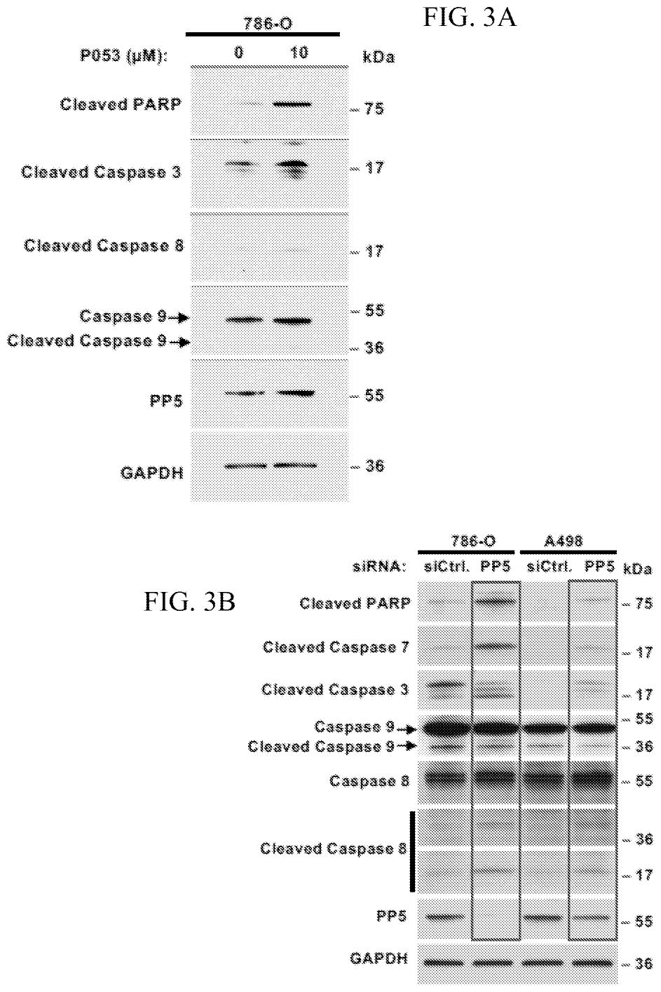

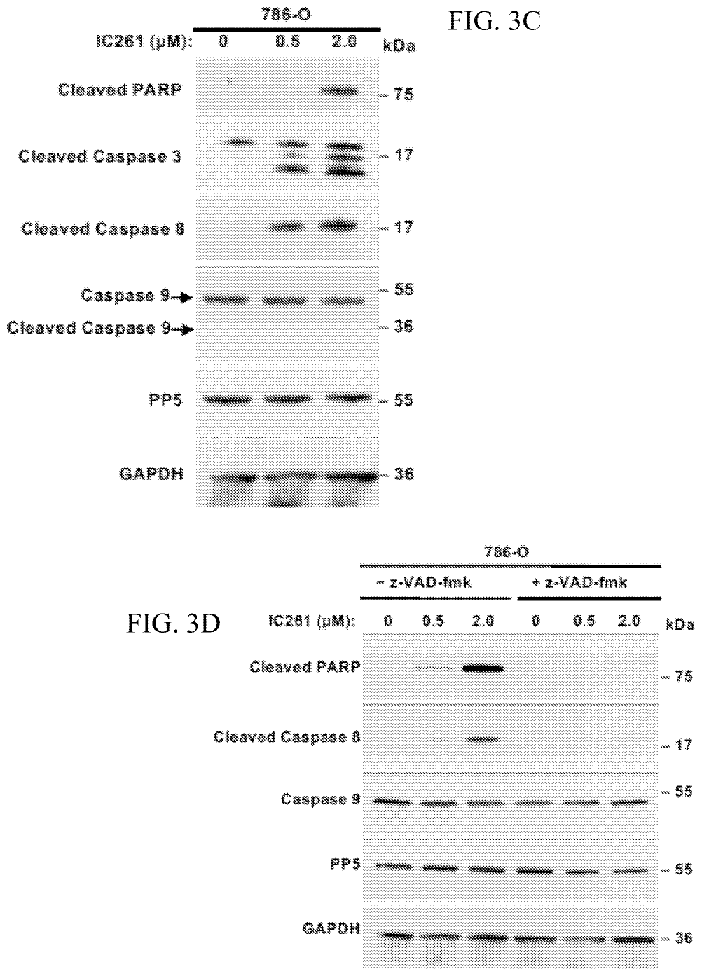

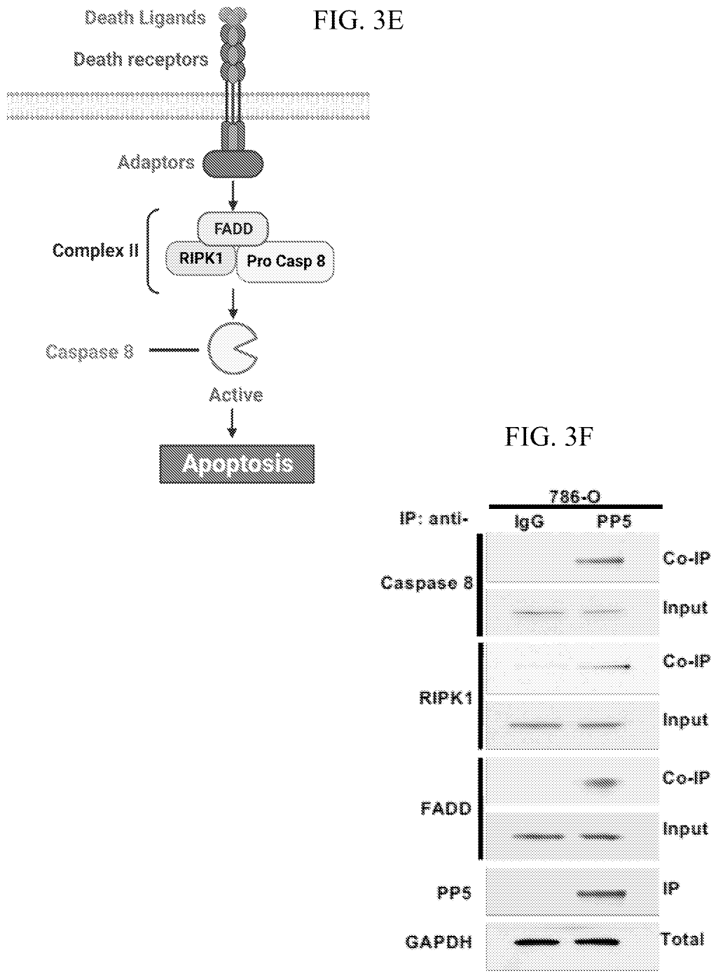

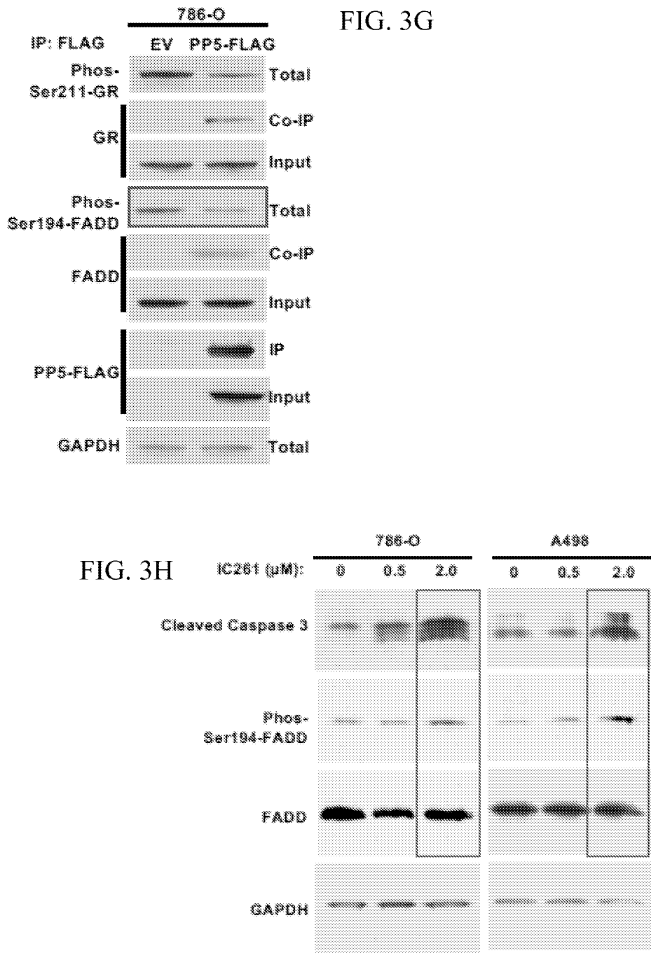

FIGS. S4A-S4F present specific small molecule inhibitor of PP5 induces apoptosis in VHL-null ccRCC (Related to FIGS. 2-3). FIG. S4A presents IC₅₀ measurements for PP5 in the presence of P5 (orange line), P13 (red line), or P053 (purple line) (n=2). Data shown as mean±S.D. FIG. S4B presents VHL protein from HEK293, HK2, Caki-1, Caki-2, A498 and 786-O cells was assessed by immunoblotting. GAPDH was used as a loading control. FIG. S4C presents Flow-cytometric assessment of cell death in Caki-1, Caki-2, A498, and 786-O following treatment with 30 μM P053 for 24 hrs (blue) or 40 μM for 24 hrs (orange), as determined by PI staining. Pink indicates no treatment (NT). FIG. S4D presents targeted siRNA was used to knockdown (KD) PP5 in 786-O cells. PP5 KD was confirmed by immunoblotting. Induction of apoptotic markers shown by immunoblotting using anti-cleaved caspase-3 and -8 and cleaved PARP antibodies. Phosphorylation level of S194-FADD was assessed by immunoblot. GAPDH was used as a control. FIG. S4E presents increased phosphorylation of S194-FADD in PP5 KO HAP1 cells was assessed by immunoblotting. GAPDH was used as a control. FIG. S4F presents WT-PP5-FLAG was transiently expressed in 786-O cells. EV was used as a control. Phosphorylation level of S161-RIPK1 and S166-RIPK1 was assessed by immunoblot. GAPDH was used as a loading control.

It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings.

### Definitions

As used in the present specification, the following words and phrases are generally intended to have the meanings as set forth below, except to the extent that the context in which they are used indicates otherwise.

As used herein, the singular forms “a”, “an”, and “the” include plural references unless the context clearly dictates otherwise. Thus, for example, references to “a compound” include the use of one or more compound(s). “A step” of a method means at least one step, and it could be one, two, three, four, five or even more method steps.

As used herein the terms “about,” “approximately,” and the like, when used in connection with a numerical variable, generally refers to the value of the variable and to all values of the variable that are within the experimental error (e.g., within the 95% confidence interval [CI 95%] for the mean) or within ±10% of the indicated value, whichever is greater.

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers to C₁₋₈ straight-chain alkyls. In other embodiments, “alkyl” refers to C₁₋₈ branched-chain alkyls. In embodiments, alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl. Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

As used herein, the term “heteroalkyl” by itself or in combination with another term means, unless otherwise stated, a stable straight or branched chain alkyl group consisting of the stated number of carbon atoms and one or two heteroatoms of O, N, and S, and wherein the nitrogen and sulfur atoms may be optionally oxidized and the nitrogen heteroatom may be optionally quaternized. In embodiments, the heteroatom(s) may be placed at any position of the heteroalkyl group, including between the rest of the heteroalkyl group and the fragment to which it is attached, as well as attached to the most distal carbon atom in the heteroalkyl group. Non-limiting examples include: —O—CH₂—CH₂—CH₃, —CH₂—CH₂—CH₂—OH, —CH₂—CH₂CH₂—NH—CH₃, and —CH₂—S—CH₂—CH₃. In embodiments, up to two heteroatoms may be consecutive, such as, for example, —CH₂—NH—OCH₃, or —CH₂—CH₂—S—S—CH₃. In embodiments, heteroalkyl groups have 1-12 carbons.

As used herein, the term “alkenyl,” denotes a monovalent group derived from a hydrocarbon moiety containing at least two carbon atoms and at least one carbon-carbon double bond. In embodiments, the double bond may or may not be the point of attachment to another group. Alkenyl groups (e.g., C₂-C₈-alkenyl) include, but are not limited to, for example, ethenyl, propenyl, prop-1-en-2-yl, butenyl, 1-methyl-2-buten-1-yl, heptenyl, octenyl and the like.

As used herein the term “apoptosis”, refers to a form of programmed cell death in multicellular organisms that involves a series of biochemical events that lead to a variety of morphological changes, including blebbing, changes to the cell membrane such as loss of membrane asymmetry and attachment, cell shrinkage, nuclear fragmentation, chromatin condensation, and chromosomal DNA fragmentation. Defective apoptotic processes have been implicated in an extensive variety of diseases; for example, defects in the apoptotic pathway have been implicated in diseases associated with uncontrolled cell proliferations, such as cancer. See e.g. U.S. Patent Publication No. 20200289513 herein entirely incorporated by reference.

A number of terms herein relate to cancer. “Cancer” is intended herein to encompass all forms of abnormal or improperly regulated reproduction of cells in a subject.

The growth of cancer cells (“growth” herein referring generally to cell division but also to the growth in size of masses of cells) is characteristically uncontrolled or inadequately controlled, as is the death (e.g., “apoptosis”) of such cells. Local accumulations of such cells result in a tumor. More broadly, and still denoting “tumors” herein are accumulations ranging from a cluster of lymphocytes at a site of infection to vascularized overgrowths, both benign and malignant. A “malignant” tumor (as opposed to a “benign” tumor) herein includes cells that tend to migrate to nearby tissues, including cells that may travel through the circulatory system to invade or colonize tissues or organs at considerable remove from their site of origin in the “primary tumor,” so-called herein. Metastatic cells are adapted to penetrate blood vessel wells to enter (“intravasate”) and exit (“extravasate”) blood vessels. Tumors capable of releasing such cells are also referred to herein as “metastatic.” The term is used herein also to denote any cell in such a tumor that is capable of such travel, or that is en route, or that has established a foothold in a target tissue. For example, a metastatic breast cancer cell that has taken root in the lung is referred to herein as a “lung metastasis.” Metastatic cells may be identified herein by their respective sites of origin and destination, such as “breast-to-bone metastatic.” In the target tissue, a colony of metastatic cells can grow into a “secondary tumor,” so called herein.

The terms “modified,” “mutant,” and “variant” (when the context so admits) refer to a gene or gene product that displays modifications in sequence and or functional properties (i.e., altered characteristics) when compared to the wild-type gene or gene product. In some embodiments, the modification includes at least one nucleotide insertion, deletion, or substitution.

The terms “carrier” and “vehicle” as used herein refer to usually inactive accessory substances into which a pharmaceutical substance is suspended. Exemplary carriers include liquid carriers (such as water, saline, culture medium, saline, aqueous dextrose, and glycols) and solid carriers (such as carbohydrates exemplified by starch, glucose, lactose, sucrose, and dextrans, anti-oxidants exemplified by ascorbic acid and glutathione, and hydrolyzed proteins.

As used herein, the term “halo” or “halogen” alone or as part of another substituent means, unless otherwise stated, a fluorine, chlorine, bromine, or iodine atom.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. In embodiments, a cycloalkyl group can be optionally partially unsaturated. In embodiments, the cycloalkyl group also can be optionally substituted with an alkyl group substituent as defined herein, oxo, and/or alkylene. In embodiments, there can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Non-limiting examples of monocyclic cycloalkyl rings include cyclopentyl, cyclohexyl, and cycloheptyl. Non-limiting examples of multicyclic cycloalkyl rings include adamantyl, octahydronaphthyl, decalin, camphor, camphane, and noradamantyl.

As used herein, the term “heterocycloalkyl” or “heterocyclyl” refers to a heteroalicyclic group including one to four ring heteroatoms each selected from O, S, and N. In embodiments, each heterocyclyl group has from 3 to 10 atoms in its ring system, with the proviso that the ring of said group does not contain two adjacent O or S atoms. In embodiments, heterocyclyl substituents may be alternatively defined by the number of carbon atoms, e.g., C₂-C₈-heterocyclyl indicates the number of carbon atoms contained in the heterocyclic group without including the number of heteroatoms. For example, a C₂-C₈-heterocyclyl will include an additional one to four heteroatoms. In embodiments, the heterocyclyl group has less than three heteroatoms. In embodiments, the heterocyclyl group has one to two heteroatoms. In embodiments, the heterocycloalkyl group is fused with an aromatic ring. In embodiments, nitrogen and sulfur heteroatoms may be optionally oxidized, and the nitrogen atom may be optionally quaternized. The heterocyclic system may be attached, unless otherwise stated, at any heteroatom or carbon atom that affords a stable structure.

As used herein, the term “aromatic” refers to a carbocycle or heterocycle with one or more polyunsaturated rings and having aromatic character, i.e., having (4n+2) delocalized π (pi) electrons, where n is an integer.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. In embodiments, the term “aryl” specifically encompasses heterocyclic aromatic compounds. The aromatic ring(s) can include phenyl, naphthyl, biphenyl, diphenylether, diphenylamine and benzophenone, among others. In particular embodiments, the term “aryl” means a cyclic aromatic including about 5 to about 10 carbon atoms, e.g., 5, 6, 7, 8, 9, or 10 carbon atoms, and including 5- and 6-membered hydrocarbon and heterocyclic aromatic rings. In embodiments, an aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl. Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto. Non-limiting examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

As generally discussed herein, a structure represented generally by the formula:

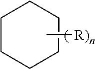

as used herein refers to a ring structure, for example, but not limited to a 3-carbon, a 4-carbon, a 5-carbon, a 6-carbon, and the like, aliphatic and/or aromatic cyclic compound, including a saturated ring structure, a partially saturated ring structure, and an unsaturated ring structure as defined herein, including a substituent R group. In embodiments, the R group can be present or absent, and when present, one or more R groups can each be substituted on one or more available carbon atoms of the ring structure. The presence or absence of the R group and number of R groups is determined by the value of the integer n. Each R group, if more than one, is substituted on an available carbon of the ring structure rather than on another R group. For example, the structure above where n is 0 to 2 would comprise compound groups including, but not limited to:

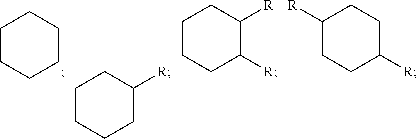

and the like.

In embodiments, a dashed line representing a bond in a cyclic ring structure indicates that the bond can be either present or absent in the ring. That is a dashed line representing a bond in a cyclic ring structure indicates that the ring structure is one of a saturated ring structure, a partially saturated ring structure, and an unsaturated ring structure.

In some embodiments, the compounds described by the presently disclosed subject matter contain a linking group. As used herein, the term “linking group” includes a chemical moiety, such as a furanyl, phenylene, thienyl, and pyrrolyl radical, which is bonded to two or more other chemical moieties, in particular aryl groups, to form a stable structure.

In embodiments, a named “R”, or “L,” group will generally have the structure that is recognized in the art as corresponding to a group having that name, unless specified otherwise herein. These definitions are intended to supplement and illustrate, not preclude, the definitions that would be apparent to one of ordinary skill in the art upon review of the present disclosure.

As used herein, the term “target activity” refers to a biological activity capable of being modulated by a selective modulator. Certain exemplary target activities include, but are not limited to, binding affinity, signal transduction, enzymatic activity, tumor growth, inflammation or inflammation-related processes, or amelioration of one or more symptoms associated with a disease or condition such as a disease or condition relating to cancer.

As used herein, the term “target protein” refers to a molecule or a portion of a protein capable of being bound by a selective binding compound. For example PP5 is a target protein for the PP5 inhibitor compositions or selective binding compounds of the present disclosure.

As used herein, the term “treatment” or “treating” is defined as the application or administration of a therapeutic agent, i.e., a one or more polycyclic compounds of the present disclosure such as one or more PP5 inhibitors, or pharmaceutically acceptable salts or solvates thereof, (alone or in combination with another pharmaceutical agent, carrier, or vehicle), to a patient, or application or administration of a therapeutic agent.

A “therapeutically effective amount” is that amount that will generate the desired therapeutic outcome (i.e., achieve therapeutic efficacy). For example, a therapeutically effective dose of a compound of the present disclosure is an amount that is sufficient to palliate, ameliorate, stabilize, reverse, prevent, slow or delay the progression of the disease state (e.g., cancer). A therapeutically effective amount can be an amount administered in a dosage protocol that includes days or weeks of administration. In certain embodiments, a therapeutically effective dose of a compound is able to improve at least one sign or symptom of a disease state. As used herein, the terms “effective amount,” and “pharmaceutically effective amount,” have the same meaning as “therapeutically effective amount”. In embodiments, a therapeutically effective amount alters the natural state of a subject.

As used herein, the term “patient,” “individual” or “subject” refers to a human or a non-human mammal. Non-human mammals include, for example, livestock and pets, such as ovine, bovine, porcine, canine, feline and murine mammals. Non-human mammals also include non-human primates, rats, rabbits and camelids. In certain embodiments, the patient, subject, or individual is human.

As used herein, the phrases “selective inhibition” or “selectively inhibit” refer to a molecule's ability to inhibit the activity or expression of a particular protein or protein isoform, or RNA, while being unable to inhibit the protein activity or expression of another protein or protein isoform, or RNA, by more than 5%. In embodiments, the PP5 inhibitors of the present disclosure selectively inhibit PP5.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of one or more of the polycyclic compounds of the present disclosure such as PP5 inhibitors, wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “solvate” refers to complexes of the compounds disclosed herein or salts thereof with solvent molecules, e.g. organic solvent molecules and/or water.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. In embodiments, there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes I-III; Celis, ed., 1994, “Cell Biology: A Laboratory Handbook” Volumes I-III; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984, “Transcription And Translation”; Freshney, ed., 1986, “Animal Cell Culture”; IRL Press, 1986, “Immobilized Cells And Enzymes”; Perbal, 1984, “A Practical Guide To Molecular Cloning.”

Before embodiments are further described, it is to be understood that this disclosure is not limited to particular embodiments described, as such may, of course, vary. It is also to be understood that the terminology used herein is for the purpose of describing particular embodiments only and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

### DETAILED DESCRIPTION OF THE EMBODIMENTS

Throughout the specification and claims, a given chemical formula or name shall encompass all optical and stereoisomers, as well as racemic mixtures where such isomers and mixtures exist.

The present disclosure provides therapeutic and preventative agents for treating or preventing diseases, disorders and conditions involving cancer. Non-limiting examples of diseases, disorders and conditions involving cancer such as kidney cancer, include one or more renal cell carcinomas (RCC) such as clear cell (ccRCC), papillary, chromophobe, collecting duct, or otherwise unclassified renal cell carcinoma. In embodiments, conditions for treatment include metastasis.

In embodiments, the present disclosure includes one or more polycyclic compounds, or pharmaceutically acceptable salts or solvates thereof, including: one or more PP5 inhibitors, such as, for example:

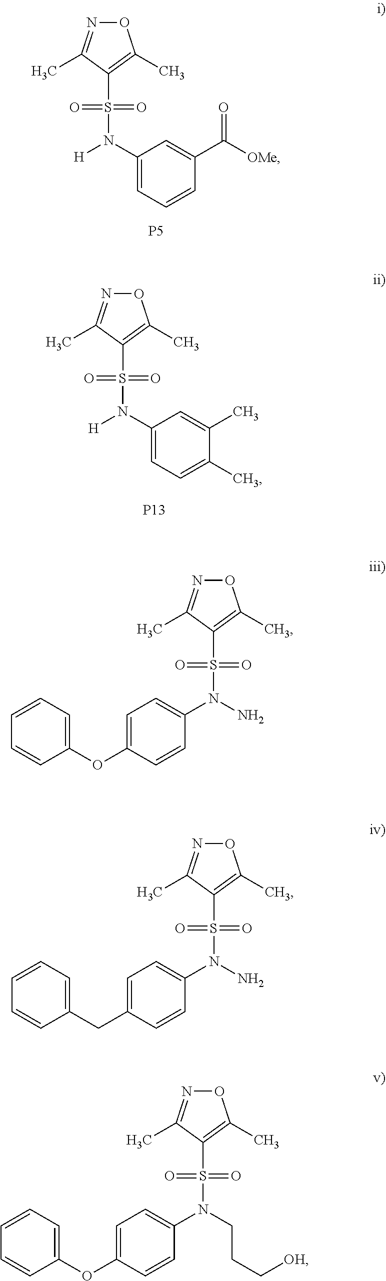

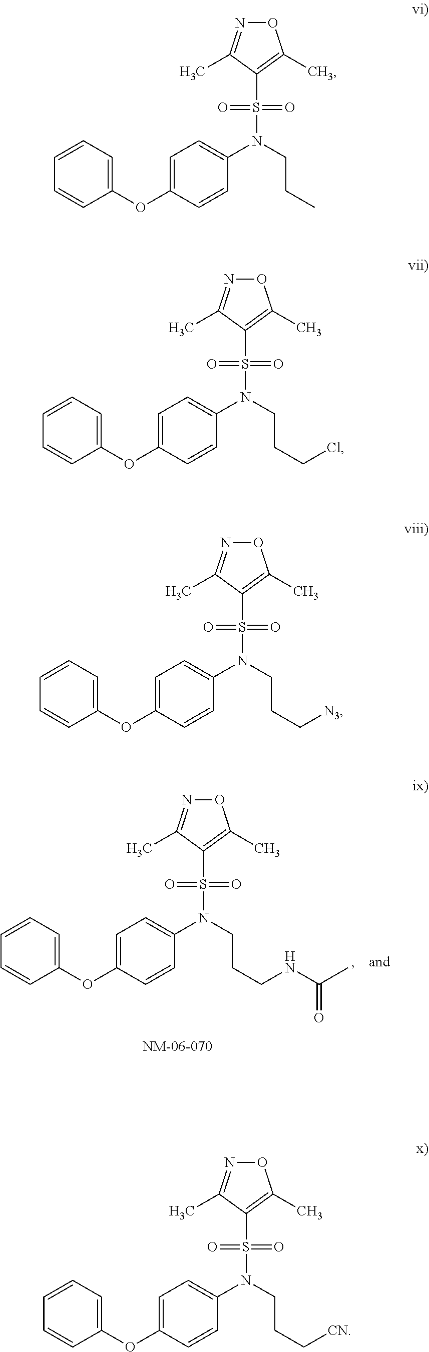 [SMILES: CCCN(c1ccc(Oc2ccccc2)cc1)S(=O)(=O)c1c(C)noc1C ; Cc1noc(C)c1S(=O)(=O)N(CCCCl)c1ccc(Oc2ccccc2)cc1 ; Cc1noc(C)c1S(=O)(=O)N(CCCN=[N+]=[N-])c1ccc(Oc2ccccc2)cc1 ; CC(=O)NCCCN(c1ccc(Oc2ccccc2)cc1)S(=O)(=O)c1c(C)noc1C ; Cc1noc(C)c1S(=O)(=O)N(CCCC#N)c1ccc(Oc2ccccc2)cc1]

In embodiments, the one or more compounds target complex Ila and/or induce the extrinsic apoptotic pathway in cancer cells. For example, the compounds of the present disclosure destabilize complex Ila, which typically includes PP5, FADD, ProCasp8, and RIPK1 constituents.

Advantages of the present disclosure include compositions and methods for treating, ameliorating, or preventing cancer. In embodiments, compositions are provided that are pharmaceutically acceptable. In embodiments, compositions of the present disclosure target, bind, and/or selectively bind to PP5. In embodiments, compositions of the present disclosure induce the extrinsic apoptotic pathway in cancer cells.

In embodiments, a compound of the present disclosure is

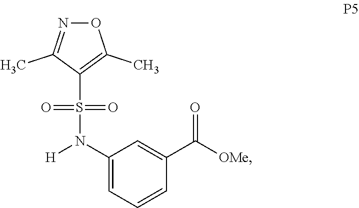 [SMILES: COC(=O)c1cccc(NS(=O)(=O)c2c(C)noc2C)c1]

or a pharmaceutically acceptable salt or solvate thereof.

In embodiments, a compound of the present disclosure is

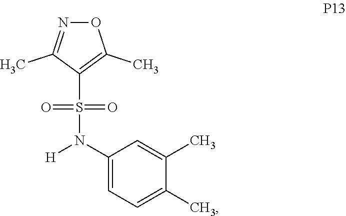 [SMILES: Cc1ccc(NS(=O)(=O)c2c(C)noc2C)cc1C]

or a pharmaceutically acceptable salt or solvate thereof.

In embodiments, a compound of the present disclosure is

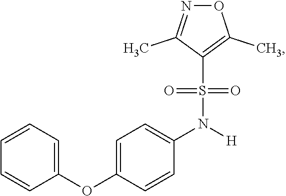 [SMILES: Cc1noc(C)c1S(=O)(=O)Nc1ccc(Oc2ccccc2)cc1]

In embodiments, a compound of the present disclosure is

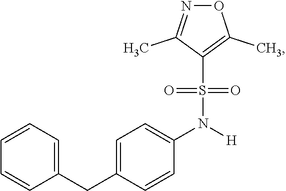 [SMILES: Cc1noc(C)c1S(=O)(=O)Nc1ccc(Cc2ccccc2)cc1]

or a pharmaceutically acceptable salt or solvate thereof.

In embodiments, a compound of the present disclosure is

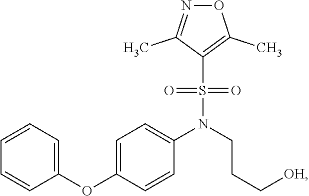 [SMILES: Cc1noc(C)c1S(=O)(=O)N(CCCO)c1ccc(Oc2ccccc2)cc1]

or a pharmaceutically acceptable salt or solvate thereof.

In embodiments, a compound of the present disclosure is

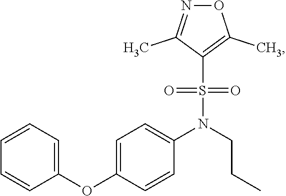 [SMILES: CCCN(c1ccc(Oc2ccccc2)cc1)S(=O)(=O)c1c(C)noc1C]

or a pharmaceutically acceptable salt or solvate thereof.

In embodiments, a compound of the present disclosure is

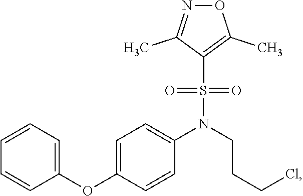 [SMILES: Cc1noc(C)c1S(=O)(=O)N(CCCCl)c1ccc(Oc2ccccc2)cc1]

or a pharmaceutically acceptable salt or solvate thereof.

In embodiments, a compound of the present disclosure is

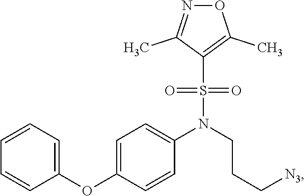 [SMILES: Cc1noc(C)c1S(=O)(=O)N(CCCN=[N+]=[N-])c1ccc(Oc2ccccc2)cc1]

or a pharmaceutically acceptable salt or solvate thereof.

In embodiments, a compound of the present disclosure is

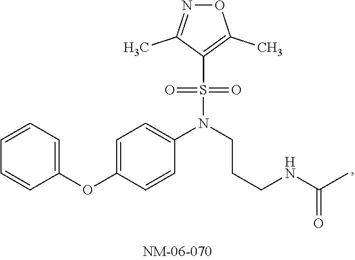 [SMILES: CC(=O)NCCCN(c1ccc(Oc2ccccc2)cc1)S(=O)(=O)c1c(C)noc1C]

or a pharmaceutically acceptable salt or solvate thereof.

In embodiments, a compound of the present disclosure is

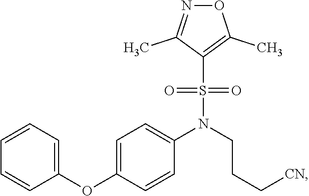 [SMILES: Cc1noc(C)c1S(=O)(=O)N(CCCC#N)c1ccc(Oc2ccccc2)cc1]

or a pharmaceutically acceptable salt or solvate thereof, and combinations thereof.

In embodiments, one or more compounds or formulas as disclosure herein is provided as a pharmaceutically acceptable salt such as derivatives of one or more of the polycyclic compounds of the present disclosure. In embodiments, a parent compound such as a polycyclic compound of the present disclosure is modified by converting an existing acid or base moiety thereof to a salt form. Examples of pharmaceutically acceptable salts include, but are not limited to, mineral or organic acid salts of basic residues such as amines; alkali or organic salts of acidic residues such as carboxylic acids; and the like. In embodiments, the pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile.

In embodiments, the polycyclic compounds of the present disclosure may possess one or more stereocenters, and each stereocenter may exist independently in either the R or S configuration.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers,” for example, diastereomers, enantiomers, and atropisomers. Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

Unless indicated otherwise, the description or naming of a particular compound in the specification and claims is intended to include both individual enantiomers and mixtures, racemic or otherwise, thereof. Within the present disclosure, any open valency appearing on a carbon, oxygen, or nitrogen atom in any structure described herein indicates the presence of a hydrogen atom. Where a chiral center exists in a structure, but no specific stereochemistry is shown for that center, both enantiomers, separately or as a mixture, are encompassed by that structure. The methods for the determination of stereochemistry and the separation of stereoisomers are well-known in the art.

In embodiments, the polycyclic compounds may exist as tautomers. All tautomers are included within the scope of the compounds presented herein.

Compounds described herein also include isotopically-labeled compounds wherein one or more atoms is replaced by an atom having the same atomic number, but an atomic mass or mass number different from the atomic mass or mass number usually found in nature. Non-limiting examples of isotopes suitable for inclusion in the compounds described herein include and are not limited to ²H or deuterium. In one embodiment, isotopically-labeled compounds are useful in drug or substrate tissue distribution studies. In another embodiment, substitution with heavier isotopes such as deuterium affords greater metabolic stability (for example, increased in vivo half-life or reduced dosage requirements).

In embodiments, the compounds described herein are labeled by other means, including, but not limited to, the use of chromophores or fluorescent moieties, bioluminescent labels, or chemiluminescent labels.

In embodiments, the compounds described herein, and other related compounds having different substituents are synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4th Ed., Vols. A and B (Plenum 2000, 2001), and Green and Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein. See also Sergey I. Filimonov, Mikhail K. Korsakov, Dmitry V. Kravchenko, Mikhail V. Dorogov, Sergey E. Tkachenko, Alexandre V. Ivachtchenko, Convenient Synthesis of Novel 5-Substituted 3-Methylisoxazole-4-sulfonamides, J. Heterocyclic Chem., 43, 663 (2006)(e.g., see the left hand column of page 664, and Scheme 1, step iii shown therein and organic synthesis described therein); Hewings, Fedorov, Filippakopoulos, Martin, Picaud, Tumber, Wells, Olcina, Freeman, Gill, Ritchie, Sheppard, Russell, Hammond, Knapp, Brennan, and Conway, Optimization of 3,5-Dimethylisoxazole Derivatives as Potent Bromodomain Ligands, Journal of Medicinal Chemistry 2013 56 (8), 3217-3227; and A. Alberola, A. M. Gonzalez, M. A. Laguna, F. J. Pulido, Synthesis of 4-Functionalized 2-Isoxazolines by Reduction of the Isoxazole Ring with Complex Metal Hydrides, Synthesis 1983; 1983(5): 413-414 DOI: 10.1055/s-1983-30361 (all of which are entirely incorporated by reference).

Compounds described herein are synthesized using any suitable procedures starting from compounds that are available from commercial sources or are prepared using procedures described herein.

In embodiments, the one or more compounds of the present disclosure, or pharmaceutically acceptable salts or solvates thereof of the present disclosure are suitable for use in treating, ameliorating, or preventing diseases, disorders and conditions involving cancer. In embodiments, compositions are provided that are pharmaceutically acceptable, and/or may cross the blood/brain barrier. In embodiments, compositions of the present disclosure target, bind, and/or selectively bind to PP5 to minimize, alleviate or end downstream deleterious aspects associated therewith and/or induce an extrinsic apoptotic pathway. Non-limiting examples of diseases, disorders and conditions involving cancer include those described herein above such as sarcomas and the like.

Provided herein are methods of treating a medical condition involving cancer in a subject in need thereof by administering to the subject an effective amount of a polycyclic compound of the present disclosure such as those identified above and in the Figures. In embodiments, the medical condition involving cancer include one or more renal cell carcinomas (RCC) such as clear cell (ccRCC), papillary, chromophobe, collecting duct, or otherwise unclassified renal cell carcinoma. Other kidney cancers may include transitional cell carcinoma, Wilms' tumor, or renal sarcoma. In embodiments, conditions for treatment include metastasis.

In some embodiments, the effective amount is an amount sufficient to block one or more harmful effects of a nonfunctional extrinsic apoptotic pathway in one or more cancer cells. In embodiment, the effective amount is an amount sufficient to enable a nonfunctional extrinsic apoptotic pathway in a cancer cell or tumor. In embodiments, pharmaceutical agents of the present disclosure are provided in a therapeutically acceptable amount, in a form characterized as pharmaceutically acceptable.

In embodiments, the present disclosure includes a method of inhibiting cancer cells or a tumor by targeting PP5 in a subject in need thereof by administering to the subject a therapeutically effective amount of a polycyclic compound of the present disclosure such as those shown above and in the accompanying figures, or a pharmaceutically acceptable salt or solvate thereof.

In embodiments, the compositions of the present disclosure are suitable for use in a pharmaceutically acceptable formulation. In embodiments, the present disclosure includes a pharmaceutical formulation including a polycyclic compound of the present disclosure, or a pharmaceutically acceptable salt, and a pharmaceutically acceptable carrier. In embodiments, a polycyclic compound of the present disclosure may be referred to as an active compound. Pharmaceutical formulations including the active compounds also are provided herein. These pharmaceutical formulations include active compounds as described herein, in a pharmaceutically acceptable carrier. Pharmaceutical formulations can be prepared for oral, intravenous, or aerosol administration. Also, the presently disclosed subject matter provides such active compounds that have been lyophilized and that can be reconstituted to form pharmaceutically acceptable formulations for administration, for example, as by intravenous or intramuscular injection. In embodiments, a therapeutically effective dosage of any specific active compound, the use of which is within the scope of embodiments described herein, will vary somewhat from compound to compound, and patient to patient, and will depend upon the condition of the patient and the route of delivery.

Administration/Dosage/Formulations: In embodiments, provided herein is a pharmaceutical composition including at least one polycyclic compound of the present disclosure, or a pharmaceutically acceptable salt or solvate thereof. Actual dosage levels of the active ingredients in the pharmaceutical compositions of this disclosure may be varied to obtain an amount of the active ingredient that is effective to achieve the desired therapeutic response for a particular patient, composition, and mode of administration, without being toxic to the patient.

In embodiments, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could begin administration of the pharmaceutical composition to dose the polycyclic compound of the present disclosure, at levels lower than that required to achieve the desired therapeutic effect and gradually increase the dosage until the desired effect is achieved.

In some embodiments, the compounds and salts disclosed herein may be administered as a solvate in a continuous manner. For example, a single dose may be administered to a subject as a solvate (e.g., intravenously or a delayed release capsule) for an administration period of 1, 2, 3, 4, 5, 6, 7, 8, 9, 10, 15, 30, 45, or 60 minutes. In some embodiments, a single dose may be administered to a subject as a solvate for an administration period of 1-5, 5-10, 10-15, 15-30, 30-45, or 45-60 minutes. In some embodiments, a single dose may be administered to a subject as a solvate for an administration period of 1-60 minutes. In some embodiments a single dose may be administered to a subject for an administration period of 1, 2, 3, 4, 5, 6, 7, 8, 9, 10, 11, 12, 24, 36, 48, 60, or 72 hours. In some embodiments a single dose may be administered to a subject for an administration period of 1-6, 6-12, 12-24, 24-48, 48-60, 60-72 hours. In some embodiments a single dose may be administered to a subject for an administration period of 1-72 hours.

In some embodiments, multiple doses of the compounds and salts described herein may be administered to a subject in a pulsatile or intermittent manner. As used herein the term “pulsatile dose regimen” or “intermittent dose regimen” refers to a dose administration regimen which includes at least two dosing cycles. Each subsequent dosing cycle is separated by a rest period from the preceding dosing cycle.

In particular embodiments, it is especially advantageous to formulate the compound in dosage unit form for ease of administration and uniformity of dosage. “Dosage unit form” as used herein refers to physically discrete units suited as unitary dosages for the patients to be treated, each unit containing a predetermined quantity of the polycyclic compound of the present disclosure calculated to produce the desired therapeutic effect in association with the required pharmaceutical vehicle. The dosage unit forms of the disclosure are dictated by and directly dependent on (a) the unique characteristics of the polycyclic compound of the present disclosure and the particular therapeutic effect to be achieved, and (b) the limitations inherent in the art of compounding/formulating such a polycyclic compound of the present disclosure for treatment in a patient.

In one embodiment, the compounds of the disclosure are formulated using one or more pharmaceutically acceptable excipients or carriers. In one embodiment, the pharmaceutical compositions of the invention comprise a therapeutically effective amount of a polycyclic compound of the present disclosure, and a pharmaceutically acceptable carrier.

Routes of administration of any of the compositions of the disclosure include oral, nasal, rectal, intravaginal, parenteral, buccal, sublingual or topical. The compounds for use in the disclosure may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For parenteral administration, the polycyclic compound of the present disclosure may be formulated for injection or infusion, for example, intravenous, intramuscular or subcutaneous injection or infusion, or for administration in a bolus dose or continuous infusion. Suspensions, solutions or emulsions in an oily or aqueous vehicle, optionally containing other formulatory agents such as suspending, stabilizing or dispersing agents may be used.

In some embodiments, the present disclosure includes a method of treating cancer, including: a) providing i) a subject with cancer, ii) one or more protein phosphatase-5 inhibitors, and b) treating said subject with the one or more PP5 inhibitors. In embodiments, treating includes administering a therapeutically effective amount of one or more protein phosphatase-5 inhibitors to the subject. In embodiments, the cancer is renal cancer such as renal cell carcinoma. In embodiments, the one or more protein phosphatase-5 inhibitors are characterized as a PP5 specific inhibitor. In embodiments, the one or more protein phosphatase-5 inhibitors are selected from the group consisting of:

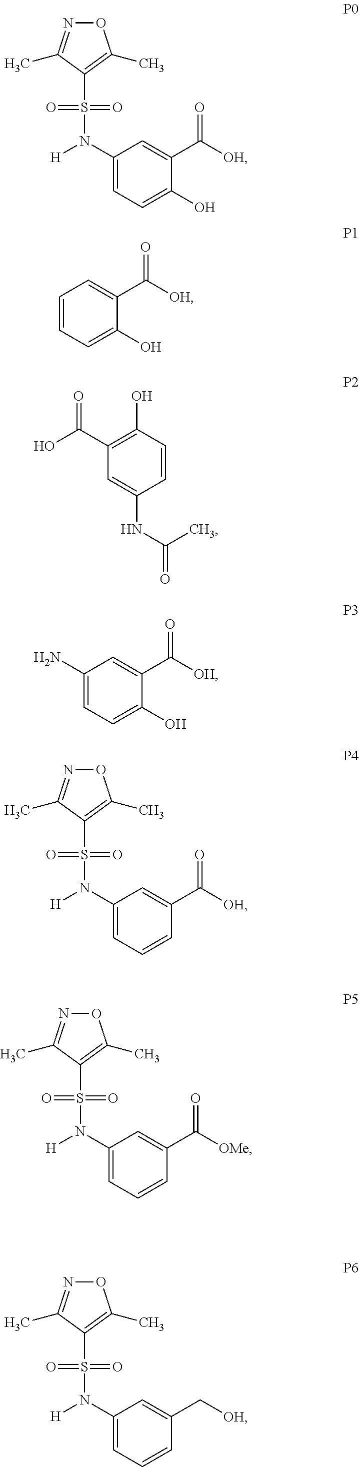

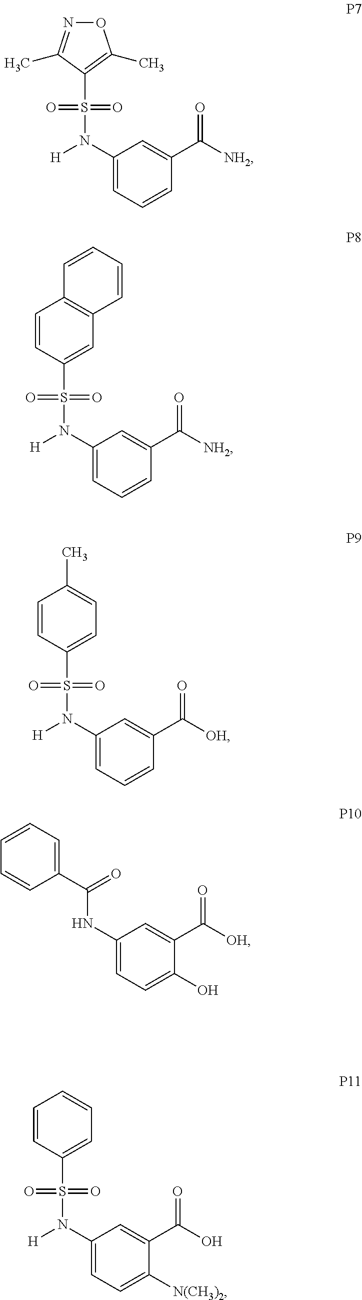 [SMILES: Cc1noc(C)c1S(=O)(=O)Nc1cccc(C(N)=O)c1 ; NC(=O)c1cccc(NS(=O)(=O)c2ccc3ccccc3c2)c1 ; Cc1ccc(S(=O)(=O)Nc2cccc(C(=O)O)c2)cc1 ; O=C(Nc1ccc(O)c(C(=O)O)c1)c1ccccc1 ; CN(C)c1ccc(NS(=O)(=O)c2ccccc2)cc1C(=O)O]

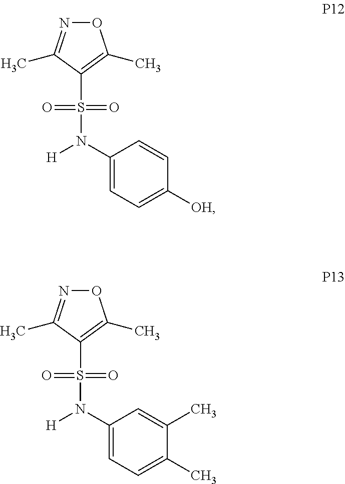

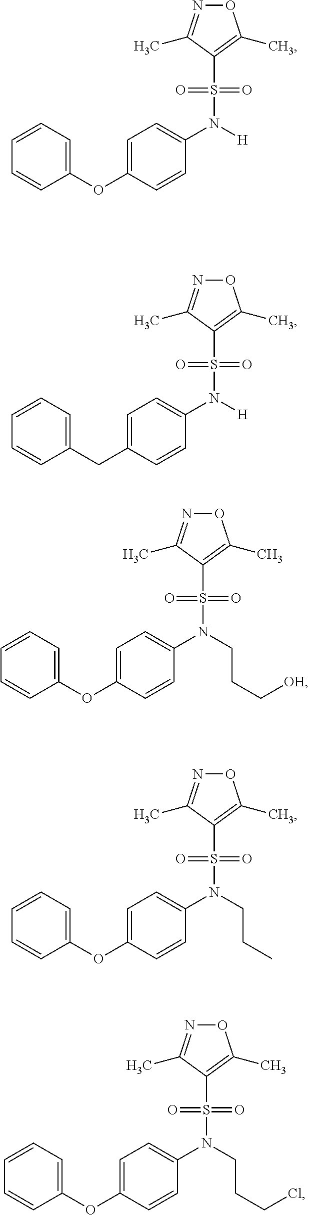 [SMILES: Cc1noc(C)c1S(=O)(=O)Nc1ccc(Oc2ccccc2)cc1 ; Cc1noc(C)c1S(=O)(=O)Nc1ccc(Cc2ccccc2)cc1 ; Cc1noc(C)c1S(=O)(=O)N(CCCO)c1ccc(Oc2ccccc2)cc1 ; CCCN(c1ccc(Oc2ccccc2)cc1)S(=O)(=O)c1c(C)noc1C ; Cc1noc(C)c1S(=O)(=O)N(CCCCl)c1ccc(Oc2ccccc2)cc1]

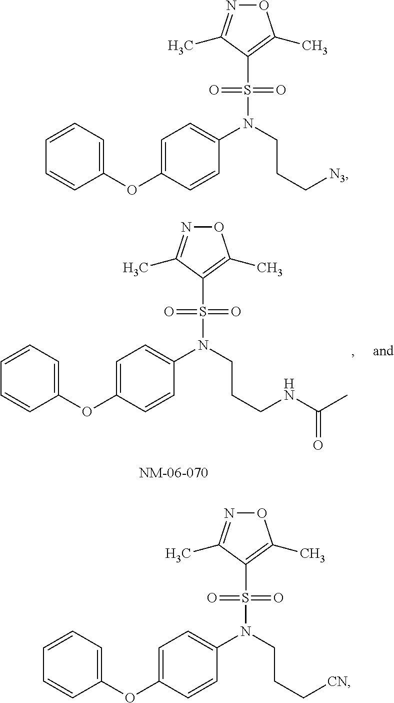

or a pharmaceutically acceptable salt thereof, and combinations thereof. In some embodiments, treating the subject with the one or more protein phosphatase-5 inhibitors is sequential. In embodiments, treating the subject with the one or more protein phosphatase-5 inhibitors is simultaneous. In embodiments, treating with the one or more protein phosphatase-5 inhibitors results in reduced proliferation of at least some of the cancer cells within said subject.

In some embodiments, the present disclosure includes a method of treating cancer, including a) providing i) a subject with cancer and one or more compounds of: selected from the group consisting of

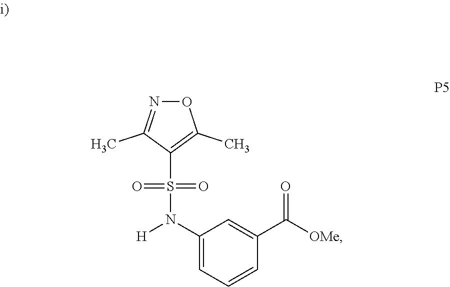 [SMILES: COC(=O)c1cccc(NS(=O)(=O)c2c(C)noc2C)c1]

or a pharmaceutically acceptable salt thereof,

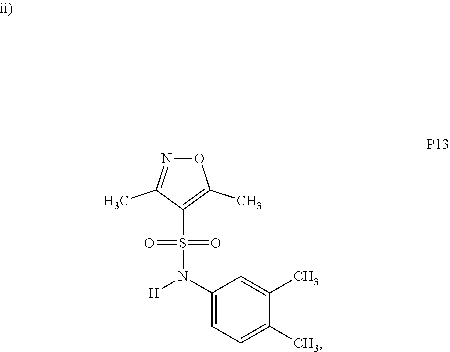 [SMILES: Cc1ccc(NS(=O)(=O)c2c(C)noc2C)cc1C]

or a pharmaceutically acceptable salt thereof,

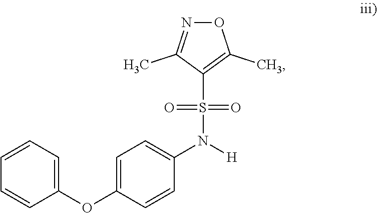 [SMILES: Cc1noc(C)c1S(=O)(=O)Nc1ccc(Oc2ccccc2)cc1]

or a pharmaceutically acceptable salt thereof,

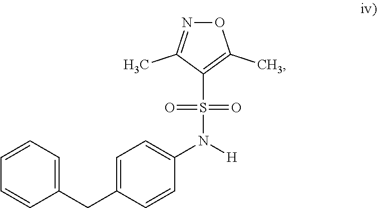 [SMILES: Cc1noc(C)c1S(=O)(=O)Nc1ccc(Cc2ccccc2)cc1]

or a pharmaceutically acceptable salt thereof,

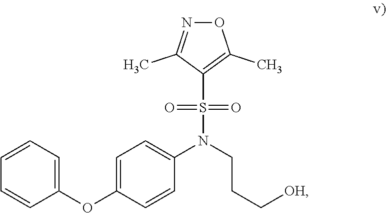 [SMILES: Cc1noc(C)c1S(=O)(=O)N(CCCO)c1ccc(Oc2ccccc2)cc1]

or a pharmaceutically acceptable salt thereof,

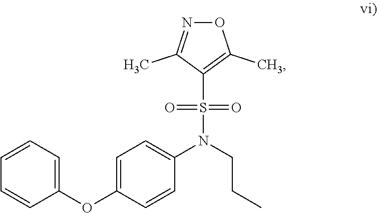 [SMILES: CCCN(c1ccc(Oc2ccccc2)cc1)S(=O)(=O)c1c(C)noc1C]

or a pharmaceutically acceptable salt thereof,

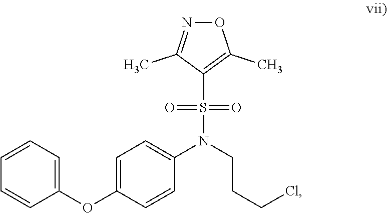 [SMILES: Cc1noc(C)c1S(=O)(=O)N(CCCCl)c1ccc(Oc2ccccc2)cc1]

or a pharmaceutically acceptable salt thereof,

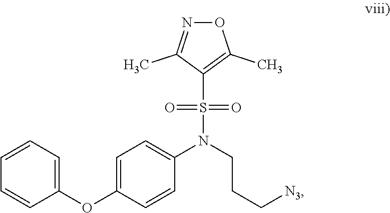 [SMILES: Cc1noc(C)c1S(=O)(=O)N(CCCN=[N+]=[N-])c1ccc(Oc2ccccc2)cc1]

or a pharmaceutically acceptable salt thereof,

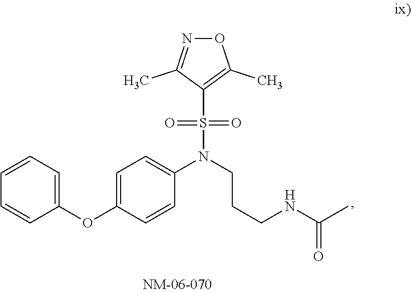 [SMILES: CC(=O)NCCCN(c1ccc(Oc2ccccc2)cc1)S(=O)(=O)c1c(C)noc1C]

or a pharmaceutically acceptable salt thereof,

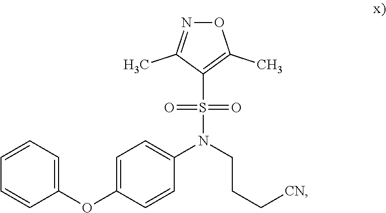 [SMILES: Cc1noc(C)c1S(=O)(=O)N(CCCC#N)c1ccc(Oc2ccccc2)cc1]

or a pharmaceutically acceptable salt thereof, and combinations thereof, and b) treating the subject with the one or more compounds or pharmaceutically acceptable salts thereof.

In some embodiments, the present disclosure includes a pharmaceutical anticancer composition including one or more PP5 inhibitors, or pharmaceutically acceptable salts thereof. In embodiments, the composition is characterized as PP5 specific. In embodiments, the one or more PP5 inhibitors are selected from the group consisting of:

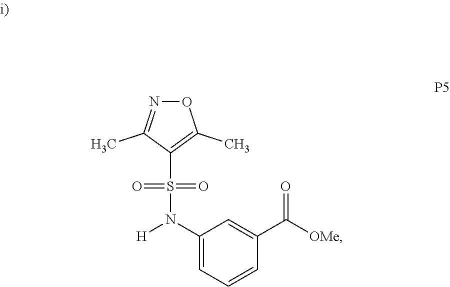 [SMILES: COC(=O)c1cccc(NS(=O)(=O)c2c(C)noc2C)c1]

or a pharmaceutically acceptable salt thereof,

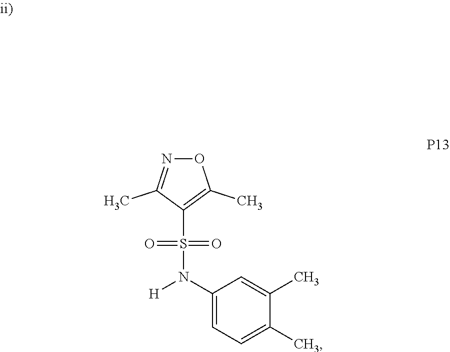 [SMILES: Cc1ccc(NS(=O)(=O)c2c(C)noc2C)cc1C]

or a pharmaceutically acceptable salt thereof,

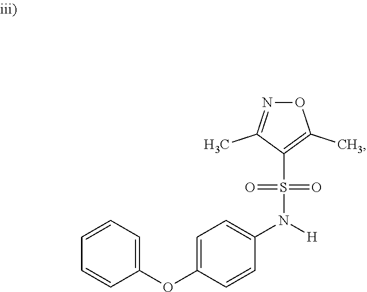 [SMILES: Cc1noc(C)c1S(=O)(=O)Nc1ccc(Oc2ccccc2)cc1]

or a pharmaceutically acceptable salt thereof,

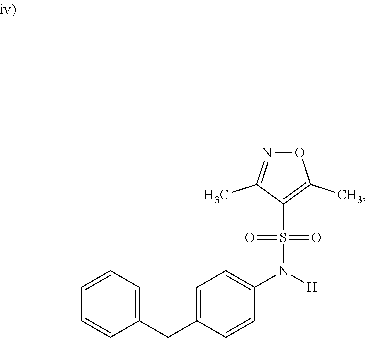 [SMILES: Cc1noc(C)c1S(=O)(=O)Nc1ccc(Cc2ccccc2)cc1]

or a pharmaceutically acceptable salt thereof,

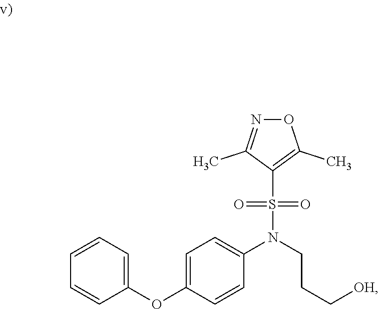 [SMILES: Cc1noc(C)c1S(=O)(=O)N(CCCO)c1ccc(Oc2ccccc2)cc1]

or a pharmaceutically acceptable salt thereof,

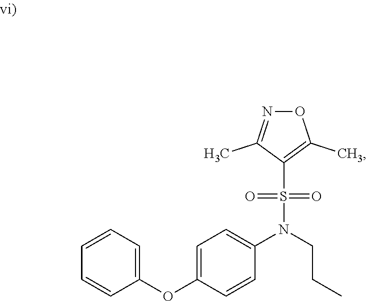 [SMILES: CCCN(c1ccc(Oc2ccccc2)cc1)S(=O)(=O)c1c(C)noc1C]

or a pharmaceutically acceptable salt thereof,

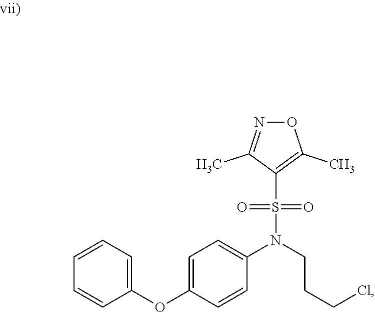 [SMILES: Cc1noc(C)c1S(=O)(=O)N(CCCCl)c1ccc(Oc2ccccc2)cc1]

or a pharmaceutically acceptable salt thereof,

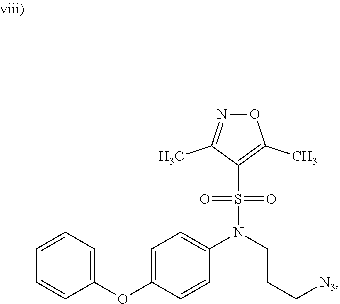 [SMILES: Cc1noc(C)c1S(=O)(=O)N(CCCN=[N+]=[N-])c1ccc(Oc2ccccc2)cc1]

or a pharmaceutically acceptable salt thereof,

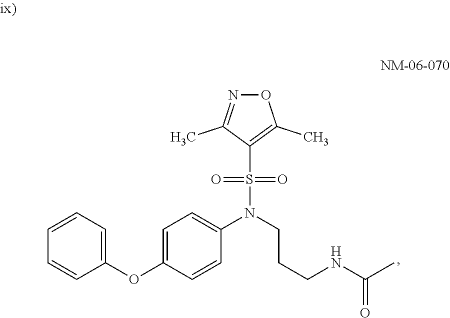 [SMILES: CC(=O)NCCCN(c1ccc(Oc2ccccc2)cc1)S(=O)(=O)c1c(C)noc1C]

or a pharmaceutically acceptable salt thereof,

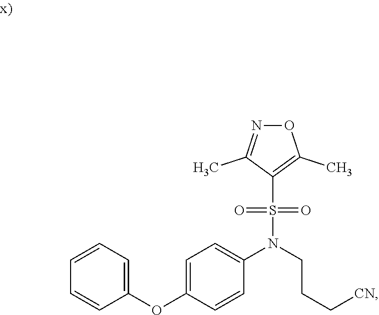 [SMILES: Cc1noc(C)c1S(=O)(=O)N(CCCC#N)c1ccc(Oc2ccccc2)cc1]

or a pharmaceutically acceptable salt thereof, and combinations thereof.

In some embodiments, the present disclosure includes a method of treating cancer, including contacting one or more cancer cells, or a cancerous tumor, with a therapeutically effective amount of one or more protein phosphatase-5 inhibitors. In embodiments, the contacting is characterized as in vivo, and activates an inactivated extrinsic apoptosis pathway.

In some embodiments, the present disclosure includes a method of treating cancer, including a) providing i) a subject with cancer, ii) one or more protein phosphatase-5 inhibitors, and b) treating said subject with the one or more PP5 inhibitors. In embodiments, treating includes administering a therapeutically effective amount of one or more protein phosphatase-5 inhibitors to the subject. In embodiments, the cancer is renal cancer. In embodiments, the renal cancer is renal cell carcinoma. In embodiments, the one or more protein phosphatase-5 inhibitors are characterized as a PP5 specific inhibitor. In embodiments, the one or more protein phosphatase-5 inhibitors are selected from the group consisting of:

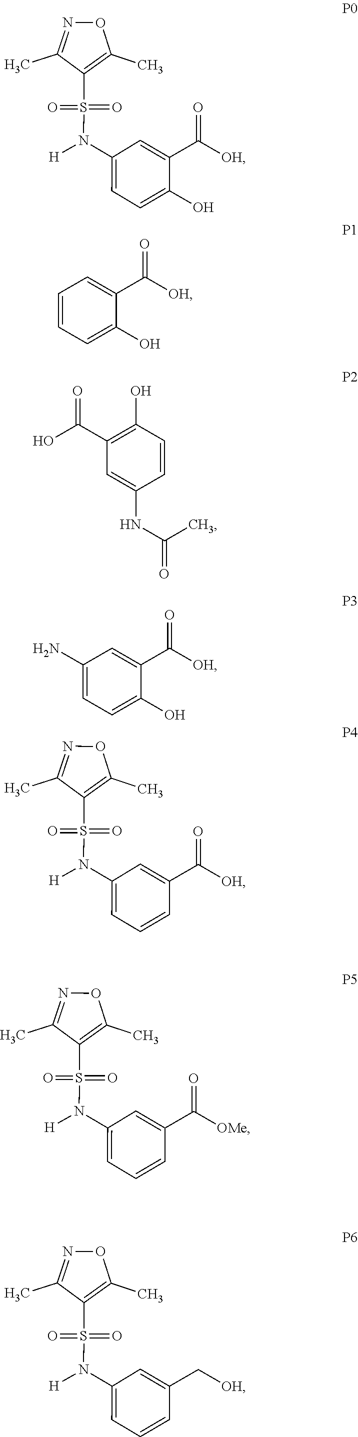

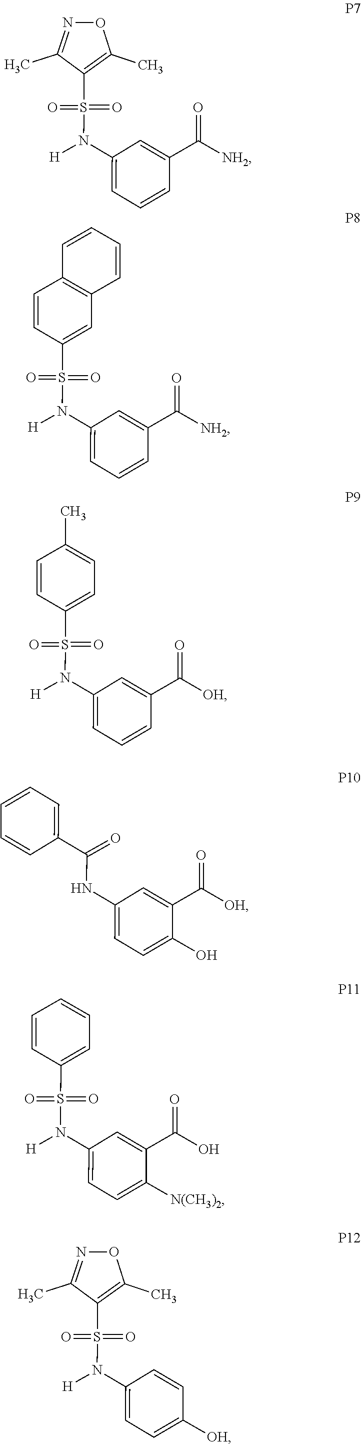

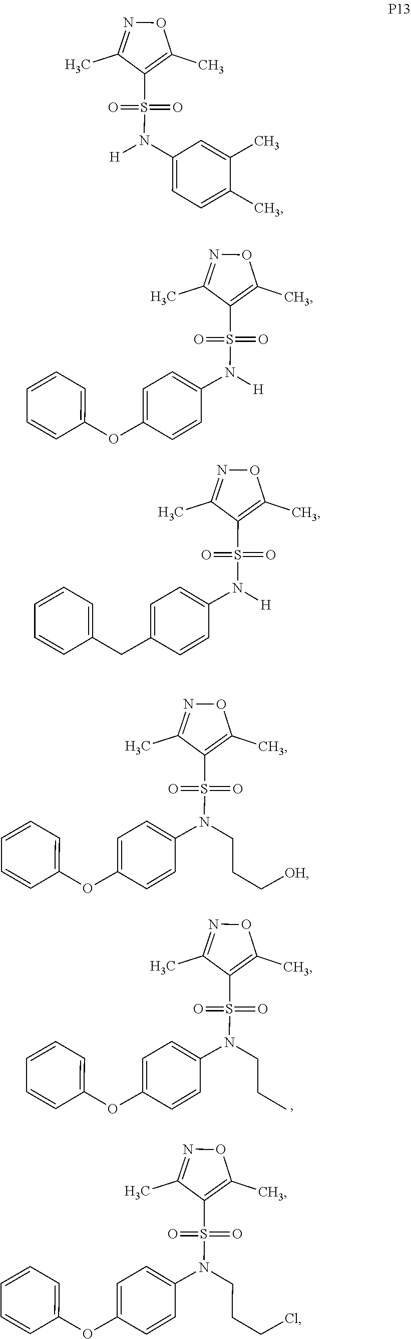

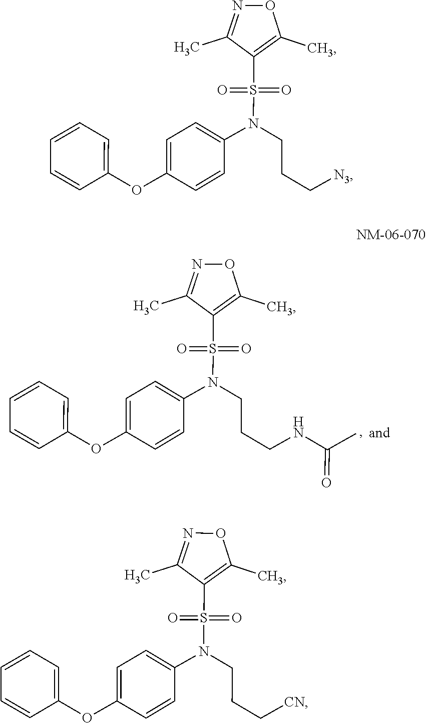 [SMILES: Cc1noc(C)c1S(=O)(=O)N(CCCN=[N+]=[N-])c1ccc(Oc2ccccc2)cc1 ; CC(=O)NCCCN(c1ccc(Oc2ccccc2)cc1)S(=O)(=O)c1c(C)noc1C ; Cc1noc(C)c1S(=O)(=O)N(CCCC#N)c1ccc(Oc2ccccc2)cc1]

and combinations thereof. In some embodiments, treating the subject with the one or more protein phosphatase-5 inhibitors is sequential. In embodiments, treating the subject with the one or more protein phosphatase-5 inhibitors is simultaneous. In embodiments, treating with the one or more protein phosphatase-5 inhibitors results in reduced proliferation of at least some of the cancer cells within said subject.

In embodiments the present disclosure includes a method of treating cancer, including: a) providing i) a subject with cancer and one or more compounds of:

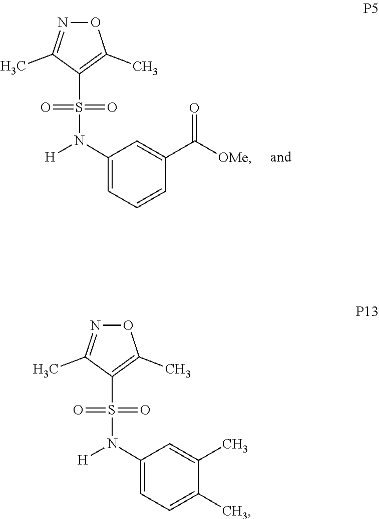

and b) treating the subject with the one or more compounds. In embodiments, the one or more compounds is provided in a therapeutically acceptable amount.

In some embodiments, the present disclosure includes a method of treating cancer, including: a) providing i) a subject with cancer and one or more compounds of:

and b) treating the subject with the one or more compounds.

In embodiments, the present disclosure includes a pharmaceutical anticancer composition including one or more PP5 inhibitors. In embodiments, the composition is characterized as PP5 specific. In embodiments, the one or more PP5 inhibitors are selected from the group consisting of:

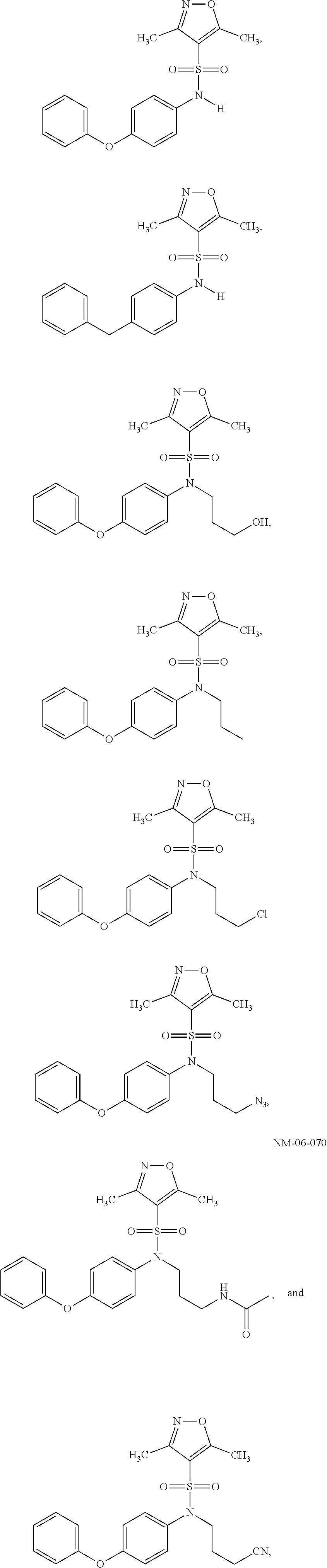

and combinations thereof.

In embodiments, the present disclosure includes a pharmaceutical anticancer composition including one or more PP5 inhibitors. In embodiments, the composition is characterized as PP5 specific. In embodiments, the one or more PP5 inhibitors are selected from the group consisting of:

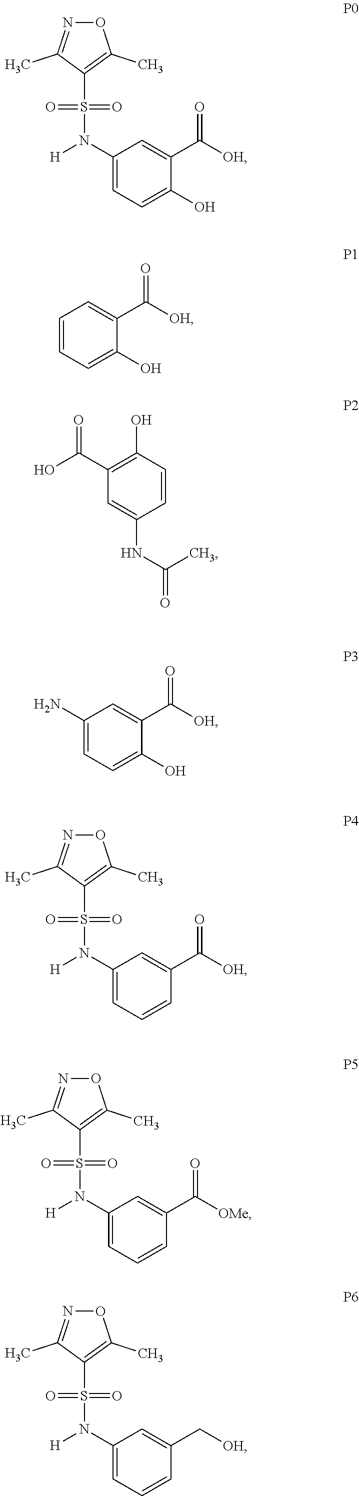

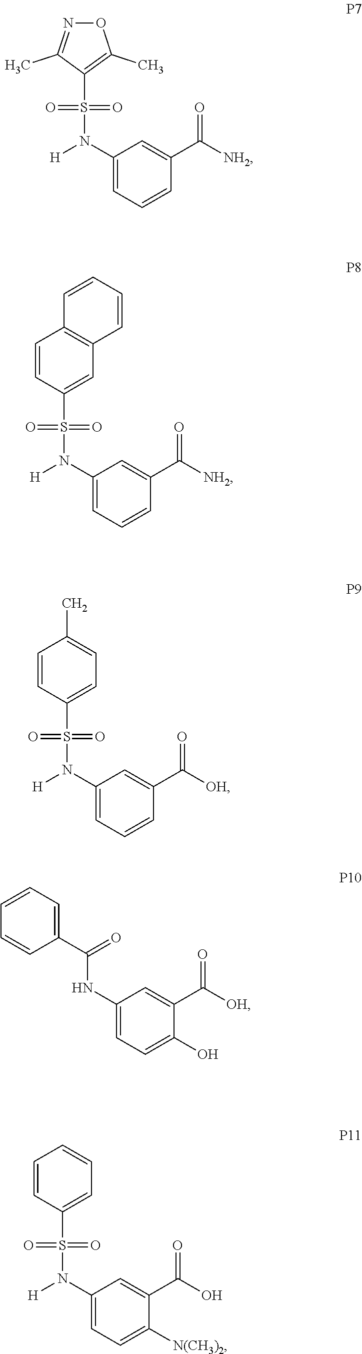 [SMILES: Cc1noc(C)c1S(=O)(=O)Nc1cccc(C(N)=O)c1 ; NC(=O)c1cccc(NS(=O)(=O)c2ccc3ccccc3c2)c1 ; Cc1ccc(S(=O)(=O)Nc2cccc(C(=O)O)c2)cc1 ; O=C(Nc1ccc(O)c(C(=O)O)c1)c1ccccc1 ; CN(C)c1ccc(NS(=O)(=O)c2ccccc2)cc1C(=O)O]

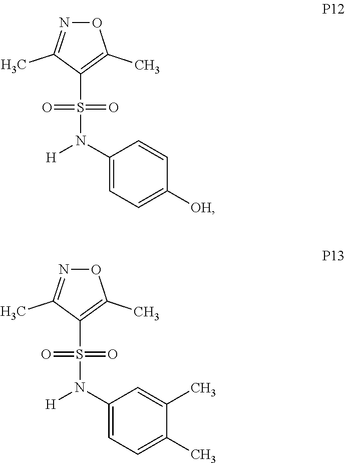

and combinations thereof.

Those skilled in the art will recognize or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, and reducing/oxidizing agents, with art-recognized alternatives and using no more than routine experimentation, are within the scope of the present disclosure.

It is to be understood that wherever values and ranges are provided herein, all values and ranges encompassed by these values and ranges, are meant to be is encompassed within the scope of the present invention. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

### DETAILED DESCRIPTION OF THE INVENTION

The serine/threonine protein phosphatase PP5 regulates several signaling cascades that are responsible for tumor initiation, progression and metastasis. Unlike other family members, a single gene encodes PP5, and its regulatory and catalytic domains are all contained within the same polypeptide. PP5 generally has low basal activity due to the interaction of the tetratricopeptide repeat (TPR) motif at its amino-terminus with the αJ-helix in the carboxy-terminus. This autoinhibitory state prevents substrates from entering the active site of PP5. Additionally, PP5 is a co-chaperone of the molecular chaperone heat shock protein-90 (Hsp90). Binding of PP5's TPR domain Hsp90 releases its autoinhibition and activates PP5. Other cellular factors such as polyunsaturated fatty acids have also been described to activate PP5 in vitro. Furthermore, we have shown that post-translational modifications of PP5 play a major is role in its switching “on” and “off” in cells. The Von Hippel-Lindau (VHL) tumor suppressor gene is the recognition subunit of an E3 ubiquitin ligase that canonically recognizes its substrates following an oxygen-dependent prolyl-hydroxylase (PHD) reaction, with hypoxia-inducible factor (HIFα) being its most-studied substrate. However, previous studies, including from our group, have demonstrated an oxygen and PHD-independent function for VHL. VHL is involved in multi-monoubiquitination and subsequent proteasomal degradation of PP5 in a hypoxia- and prolyl-hydroxylation-independent manner in normal cells therefore providing an “off” switch for PP5. The most common type of kidney cancer, clear cell renal cell carcinoma (ccRCC), is closely associated with mutations and inactivation of the VHL tumor suppressor gene. We have previously shown that VHL-deficient renal cancer cell lines and patient-derived ccRCC tumors exhibit elevated PP5 levels. Additionally, casein kinase 1 δ (CK1δ)-mediated phosphorylation of T362 in the catalytic domain of PP5 activates this phosphatase. Pharmacological inhibition of CK1δ or down-regulation of PP5 induced apoptosis and reduced proliferation in VHL-null ccRCC cells, suggesting a prosurvival role for PP5 in kidney cancer. The mechanism of PP5-dependent cell survival and whether PP5 can be directly targeted, however, remains elusive.

In this study we used in silico structure-based drug discovery approach and identified a small molecule (P053) that specifically binds and inhibits PP5 activity. Inhibition of PP5 in VHL-null ccRCC cells leads to induction of the extrinsic apoptotic pathway. We found that PP5 interacts with the extrinsic apoptotic complex II members FADD, RIPK1, and caspase 8 and dephosphorylates S194-FADD in an Hsp90 independent manner. Inhibition or down-regulation of PP5 leads to disassociation of this complex, cleavage of caspase 8 and activation of the extrinsic apoptotic pathway in ccRCC.

Characterization of PP5 specific inhibitors: Since PP5 plays a major role in survival of ccRCC, we sought to design, develop, and test specific small molecule inhibitors of this phosphatase 3. In order to identify small molecule antagonists of PP5 an in silico docking study was initiated using our previously solved X-ray crystal structure of the PP5 active site (PDB:5HPE) 6. The PP5 structure allowed us to employ a virtual screening strategy to identify new inhibitors of PP5 (FIG. 1A). Docking was performed three times with three different structures, the only differences in the X-ray structures being the nature of the metal ion bound in the active site; Mn2+ for PDB:3H60, Zn2+ for PDB:3H68 and Fe2+ was substituted for the Zn2+ ion in the PDB:3H68 structure 27 (FIGS. 1A and S1A). The threefold docking was performed due to the promiscuity of PP5 when it comes to metal ion in the active site, which can be Zn, Mn or Fe based. The active site residues of D271, N303, H304, M309 and W386 were chosen to define the site for docking. Conducting a virtual screen with the Zinc library of drug like compounds (˜3.7 million compounds) 28,29 and DOCK Blaster 30 provided a set of 200 compounds for each crystal structure that were predicted to bind to the active site of PP5. Interestingly, only one compound (P0) was found to overlap between two of these hit-sets (Mn2+ and Zn2+) despite the significant similarity in the crystal structures (FIG. S1B).

We first treated the ccRCC cell line 786-O with the overlapping hit compound P0 and showed a dose dependent increase in phosphorylation of the known PP5 substrates phospho-S13-Cdc37 and phospho-S211-GR (FIG. S1C). This suggested inhibition of PP5-mediated dephosphorylation of these known substrates. Based on the structure of P0, a number of similar molecules were purchased and assayed (FIG. S1D, compounds P1-P13) to determine which section of the molecule contained the pharmacophore. 786-O cells were treated with 20 μM compounds P0-P13 for 18 hours, and induction of apoptosis and inhibition of PP5 was assessed by immunoblotting (FIG. S1E). Compounds P4 to P13 led to marked increases in cleaved caspase 3 (indication of apoptosis) and elevated phosphorylation of PP5 substrates phospho-S13-Cdc37 and phospho-S211-GR (indication of PP5 inhibition) (FIG. S1E). We next examined the effect of varying doses of compounds P4 to P13 on proliferation of ccRCC cells by the 3-(4,5-dimethylthiazolyl-2)-2,5-diphenyltetrazolium bromide (MTT) assay (FIG. S1F). Compounds P5 and P13 significantly inhibited the proliferation of 786-O cells (FIG. 1B) and were therefore selected for further evaluation. These compounds were resynthesized and their structures were confirmed by 1H NMR, 13C NMR and high-resolution mass spectrometry (FIGS. S2A-D). We further examined the effect of P5 and P13 on 786-O cells by immunoblotting and demonstrate cleavage of the apoptotic markers caspase 3 and 8 and PARP (FIG. 1C). Taken together, we have identified two potential PP5 small molecule inhibitors that led to induction of apoptosis in ccRCC cells.

PP5 inhibitors specifically bind to the phosphatase-catalytic domain: We next sought to measure the inhibition of PP5 activity in vitro with P5 and P13. This was done using custom synthesized phospho-S211-glucocorticoid receptor (GR) peptide as a specific substrate and measuring PP5 activity by assessing free phosphate release as a result of PP5-mediated peptide dephosphorylation. As expected, our enzyme kinetics confirmed P5 and P13 to be competitive inhibitors of PP5 (FIG. 1D) with Ki of P5=277±50 nM and P13=234±50 nM. The binding data are summarized in FIG. 1D. To obtain further evidence to support the binding of P5 and P13 compounds to PP5 in vivo, we synthesized biotinylated-P5 and biotinylated-P13 (FIGS. S2E and S2F). For compound P5, this was accomplished by hydrolysis of the ester on P5, amide formation with a piperazine linker, and attachment of biotin to the distal piperazine nitrogen (FIG. S2E). In the case of compound P13, an amminopropyl linker was added to the sulfonamide nitrogen, with biotin then being attached to the far end of the linker (FIG. S2F). We next transiently expressed PP5-FLAG in HEK293 cells and then challenged the protein lysate with different amounts of biotin-P5 and biotin-P13 (FIG. 1E). Our data suggest that PP5-FLAG binds to biotinylated P5 and P13. Furthermore, at 10 μM biotin-P13 bound more to PP5-FLAG compared to biotin-P5 (FIG. 1E). Due to the improved binding and activity of P13 relative to P5 we chose to continue further characterization with only P13.

We then mutated docking site residues in the active site of PP5 to obtain further insight into the mechanism of P13 binding to PP5. These included H304Q (catalytically inactive), M309C (prevents dephosphorylation of substrate phospho-Ser13-Cdc37), and W386F (hyperactivity against substrates) as well as additional mutants which help coordinate substrate binding based on our previously published work: R275A, R400A and Y451F (FIG. 1F) 6. These mutants were transiently expressed in HEK293 cells, and then the protein lysates were challenged with different amounts of biotin-P13 (FIG. 1G). Our data showed that H304Q, M309C and W386F mutants were unable to bind to biotin-P13, confirming the P13 binding sites within the PP5 protein (FIG. 1G). Additionally, the R400A mutant had reduced binding to biotin-P13 compared to the wild-type (WT) PP5 while R275A and Y451F had increased binding (FIG. 1G).

We next synthesized BODIPY-labelled version of compound P13 (P13-BODIPY) in order to obtain the binding affinity of this inhibitor to PP5 in vitro (FIG. S3A). This was accomplished by coupling the azidopropyl-P13 10 with 4,4-difluoro-5,7-dimethyl-4-bora-3a,4a-diaza-s-indacene-3-propionic acid (BODIPY Acid 13). Using fluorescence anisotropy with recombinant PP5 protein (FIG. S3B), we determined the binding affinity of P13 compound to PP5 to be 376.2±60 nM (FIG. 1H).

Design, synthesis and characterization of a more potent PP5 inhibitor: Next used an in silico docking strategy to design series of small molecule inhibitors of PP5 based on the structure of P13 as a starting point. Examination of the docking pose of compound P13 in the active site of PP5 where the isoxazole is coordinating to the metal ions yielded some ideas about modifications that could be beneficial for binding. Neighboring the binding site was a nonpolar pocket near W386 and M309, which might accommodate a tethered nonpolar group off the xylene ring of P13. Additionally, a more polar pocket near R400 and D388 might also be accessible for an alcohol or amide tethered through the sulfonamide nitrogen of P13. Using this model as a guide, a number of sulfonamide analogs were designed and docked into the PP5 active site using Autodock Vina. This docking gave a number of potential new PP5 inhibitors that were predicted to bind to the active site of PP5 with greater affinity than the parent P13 compound. This led to synthesis of a series of compounds: P052 (16), P053 (20), P058 (22), P059 (17), P062 (18), P070 (19), P075 (23) and P129 (21) (FIG. S3C). Compounds 16 and 20 were synthesized from the addition of the respective aniline to the isoxazole sulfonyl chloride 15 (FIGS. S3D-F). Further modification of the system was accomplished by alkylation with 3-chloropropyl p-toluenesulfonate to provide the alkyl chloride 17. The chloride 17 could then be displaced with sodium azide to access azide 18, which was then reduced to the amide and acylated to provide acetamide 19. Alcohol 21 and sulfonamide 22 were prepared by alkylation of sulfonamide 16 with the appropriate alkyl halide, while nitrile 23 was prepared by displacement of alkyl chloride 17 with sodium cyanide.

We next treated the ccRCC cell line 786-O with 10 μM of the compounds shown in FIG. S3C for 18 hours and confirmed that they inhibit PP5 as evidenced by increased phosphorylation of S211-GR, which is a known substrate of PP5 (FIG. 2A). We next showed that only compounds P053, P058, P059 and P075 had the ability to induce apoptosis in 786-O cells (FIG. 2B). We then treated 786-O cells with different amounts of P053, P058, P059, P075 as well as the parent compound P13 for 18 hours and measured proliferation by MTT assay (FIG. 2C). Compound P053 significantly inhibited the proliferation of 786-O cells compared to the other compounds including P13 (FIG. 2C). Using our in vitro PP5 phosphatase specific assay we showed that P053 also is a competitive inhibitor of PP5 with Ki of 244±50 nM (FIG. 2D and FIG. S4A). Predicted P053 binding compared to P5 and P13 within PP5 protein structure is also demonstrated (FIG. 2E). We next challenged the protein lysate from 786-O cells with 1 μM biotin-P5 and P13 followed by competition with 1 μM P053 compound (FIG. 2F). Our data showed that 1 μM P053 can compete and completely displace PP5 from biotin-P5 and P13 (FIG. 2F). This suggests higher affinity of P053 towards PP5. It is also noteworthy that biotin-P5 and P13 did not bind to PP2A suggesting their specificity towards PP5 (FIG. 2F). Our current, as well as previously published work 2, has shown that PP5 plays a pro-survival role specifically in VHL-null ccRCC cells. We further demonstrated here that P053 significantly inhibited cell proliferation in VHL-null ccRCC cells, 786-O and A498 compared to normal epithelial renal cell HK2 as well as VHL-positive ccRCC cell lines Caki-1 and Caki-2 (FIG. 2G and FIG. S4B). Using propidium iodide (PI) staining of the ccRCC cells treated with different amounts of P053 compound we confirmed that P053 selectively caused cell death in VHL-null ccRCC cells in a dose dependent manner (FIG. 2H and FIG. S4C). Taken together, we have designed, and synthesized a highly specific PP5 inhibitor that caused induction of apoptosis in VHL-null ccRCC.

PP5 attenuation induces extrinsic apoptosis in clear cell renal cell cancer: Our data obtained here as well as our previous work showed that pharmacologic inhibition or silencing of PP5 in VHL-null ccRCC induced apoptosis². Given this, we asked whether the observed apoptosis resulted from the intrinsic or extrinsic signaling pathway. First, treatment of VHL-null ccRCC lines 786-O with P053 caused increased cleavage of the executioner caspase 3 and the downstream target poly-ADP ribose polymerase (PARP), hallmarks of generalized cell death (FIG. 3A). Interestingly, cleavage of caspase 8 but not caspase 9 indicated that PP5 inhibition resulted in activation of extrinsic apoptosis (FIG. 3A). We obtained similar results with small interfering RNA (siRNA) mediated knock-down (KD) of PP5 in the VHL-null ccRCC lines 786-O and A498 (FIG. 3B). T362-PP5 phosphorylation by CK1δ is coupled to its activity². We have shown that inhibition of CK1δ using the small molecule IC261 decreased PP5 activity and induced apoptosis in VHL-null ccRCC cell lines². Accordingly, treatment of 786-O cells with IC261 induced extrinsic apoptosis in a dose-dependent manner (FIG. 3C). Inhibition of caspase activity using the pan-caspase inhibitor z-VAD-fmk reversed this effect (FIG. 3D). Our data here suggests that KD or inhibition of PP5 activates the extrinsic apoptotic pathway in VHL-null ccRCC.

PP5 dephosphorylates FADD: To determine which aspect of extrinsic apoptosis is regulated by PP5, we examined PP5 association with extrinsic apoptosis proteins Fas-Associated by Death Domain (FADD) and Receptor Interacting Serine/Threonine Kinase 1 (RIPK1). These two proteins, along with caspase 8, comprise complex II of the extrinsic apoptotic pathway (FIG. 3E). We found that PP5 interacts with the members of complex II (FIG. 3F). Immunoprecipitation (IP) of PP5 from 786-O cells demonstrated co-IP of FADD, RIPK1, and caspase 8 (FIG. 3F).

Phosphorylation of S194-FADD is important for its pro-apoptotic activity, therefore we hypothesized that PP5 targets and dephosphorylates S194-FADD to suppress apoptosis. Indeed, we found that overexpression of PP5 in 786-O cells led to decreased S194-FADD phosphorylation (FIG. 3G). The bona fide PP5 substrate glucocorticoid receptor (S211-GR) has been included as a control for PP5 activity (FIG. 3G). In further support of FADD as a PP5 substrate, siRNA-mediated KD of PP5 in these cells led to increased phosphorylation of S194-FADD (FIG. S4D). Additionally, we made a similar observation of increased phosphorylation of S194-FADD in CRISPR/Cas9 mediated-knockout (KO) of PP5 in HAP1 cells (FIG. S4E). In agreement with these findings, inhibition of PP5 activity in 786-O and A498 cells using IC261 led to an increase in S194-FADD phosphorylation (FIG. 3H). Of note, overexpression of PP5-FLAG does not appear to impact the phosphorylation of S161-RIPK1 and S166-RIPK1 (FIG. S4F). Taken together, our findings here indicate that PP5 can potentially regulate extrinsic apoptosis by dephosphorylating FADD.

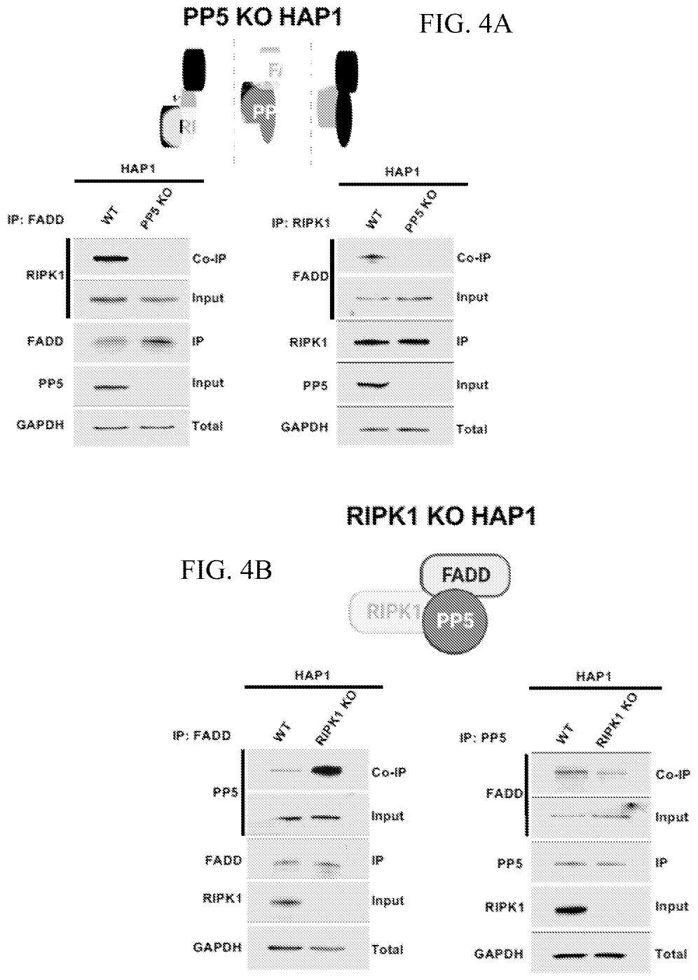

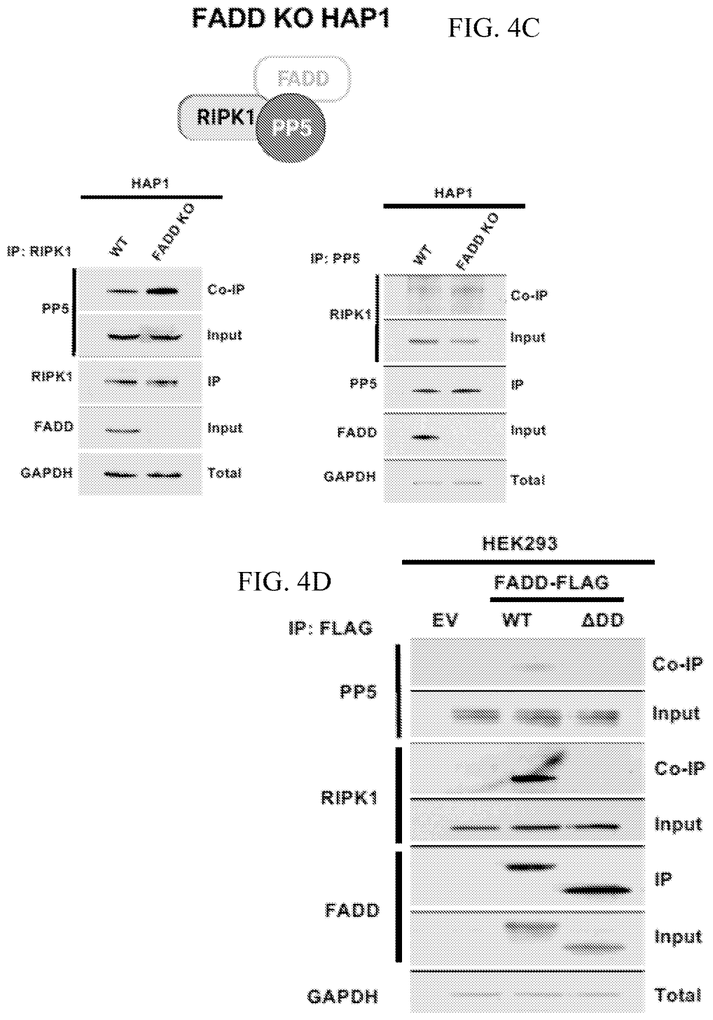

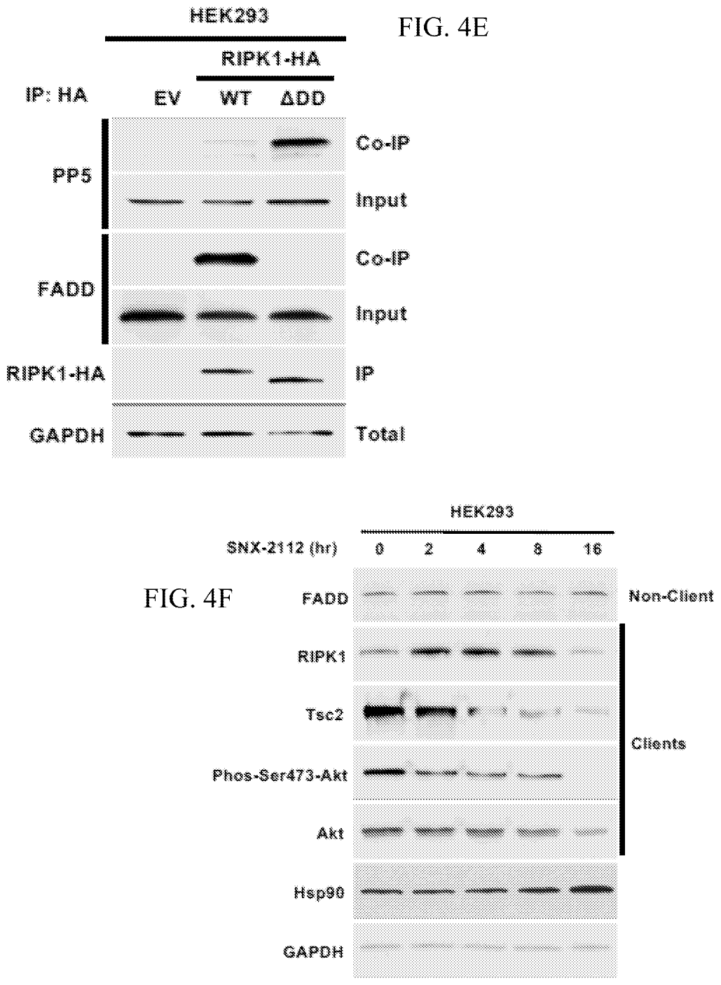

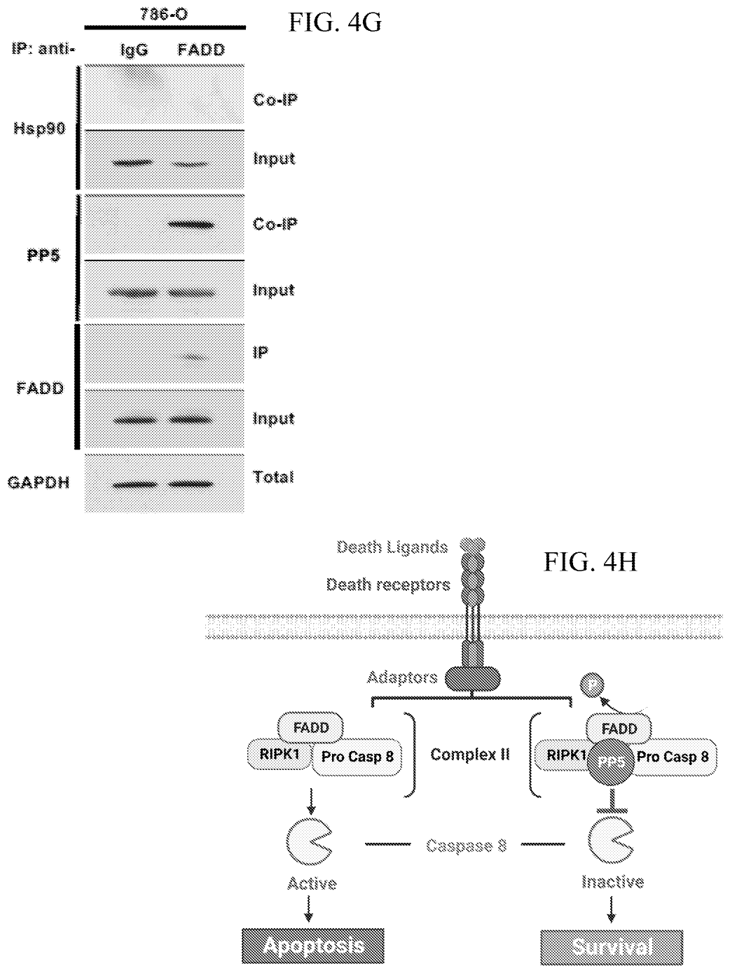

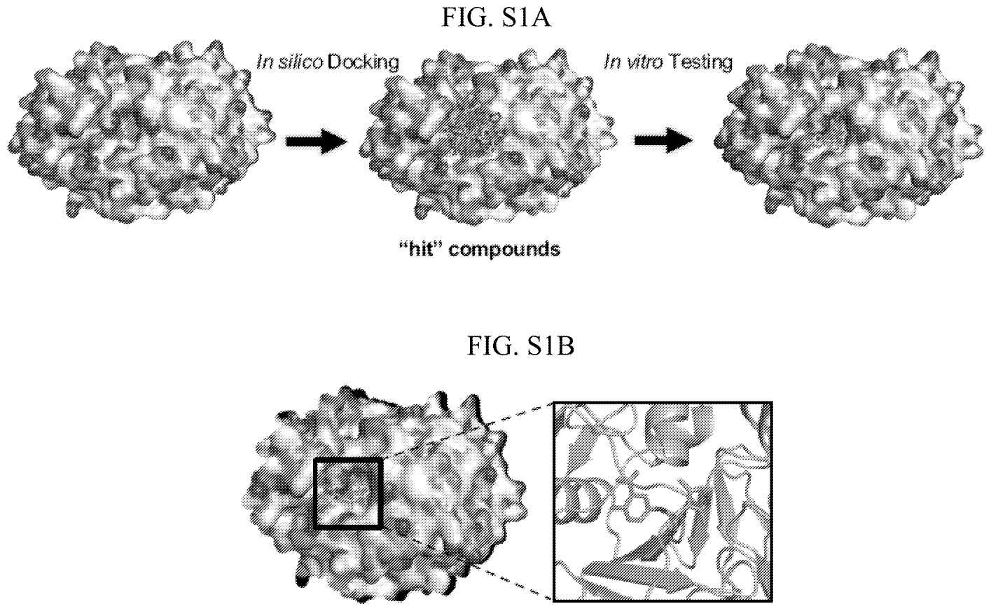

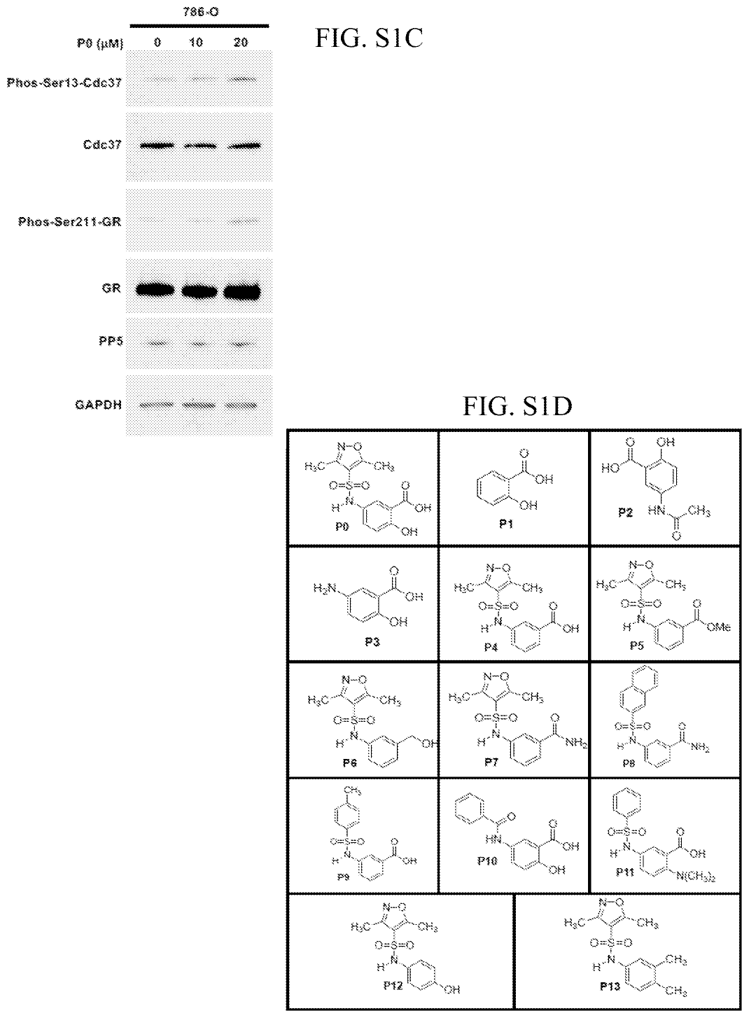

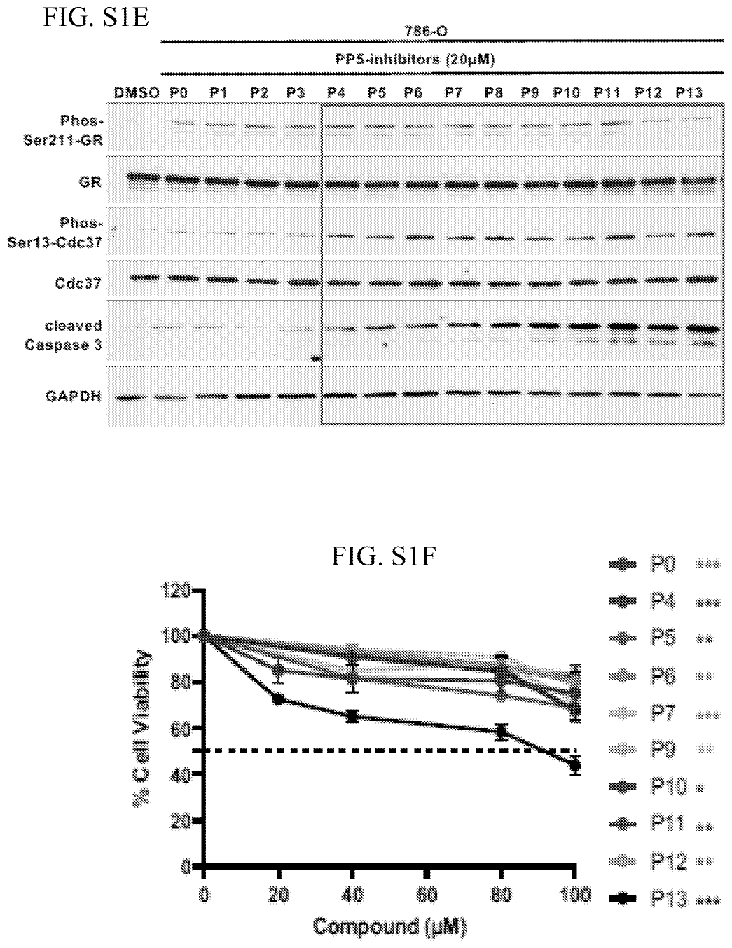

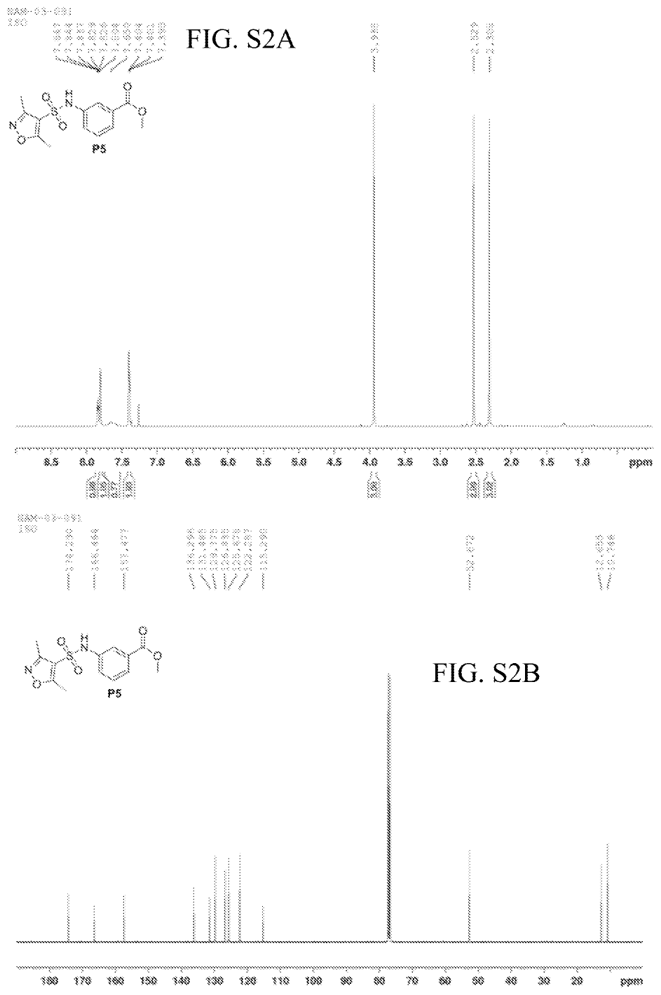

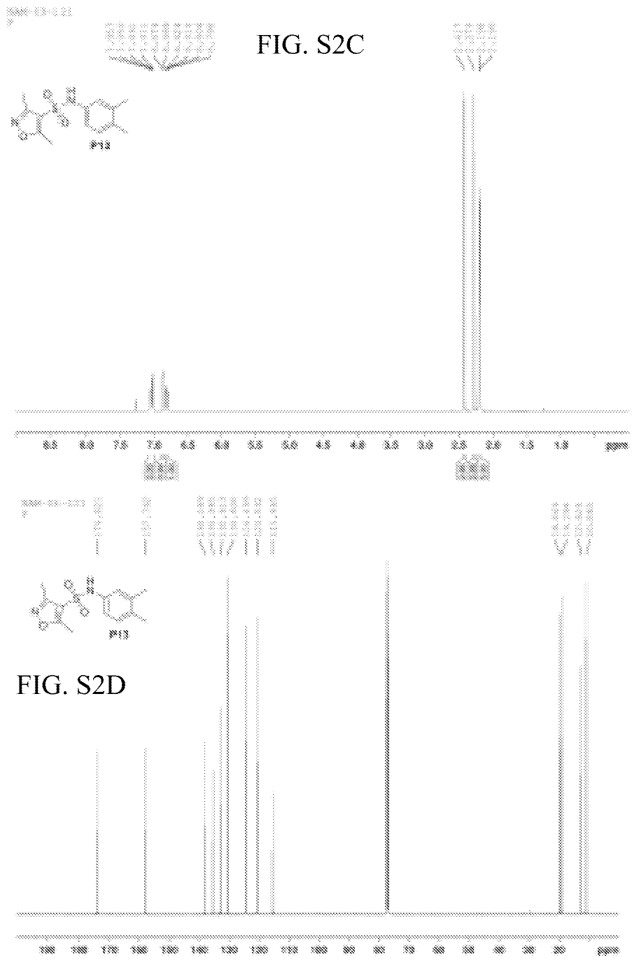

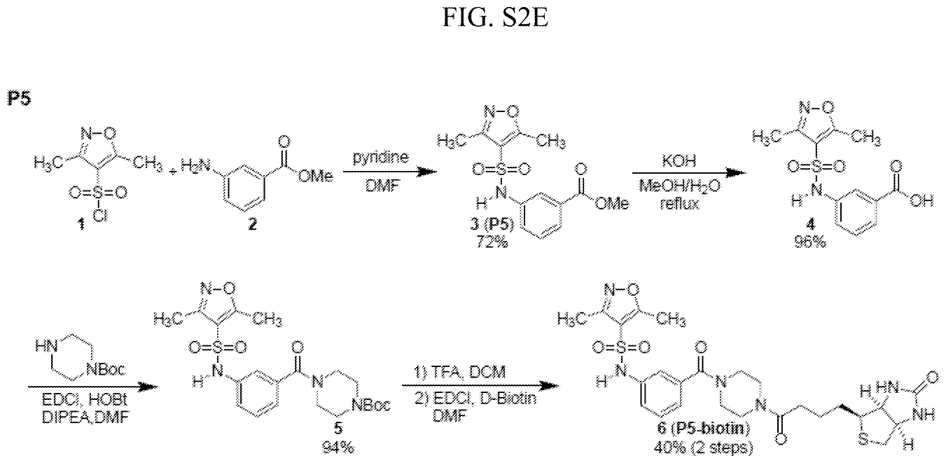

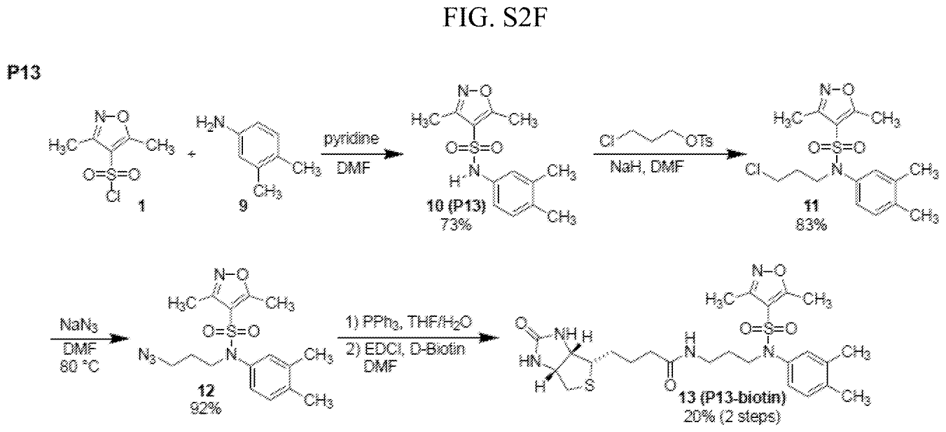

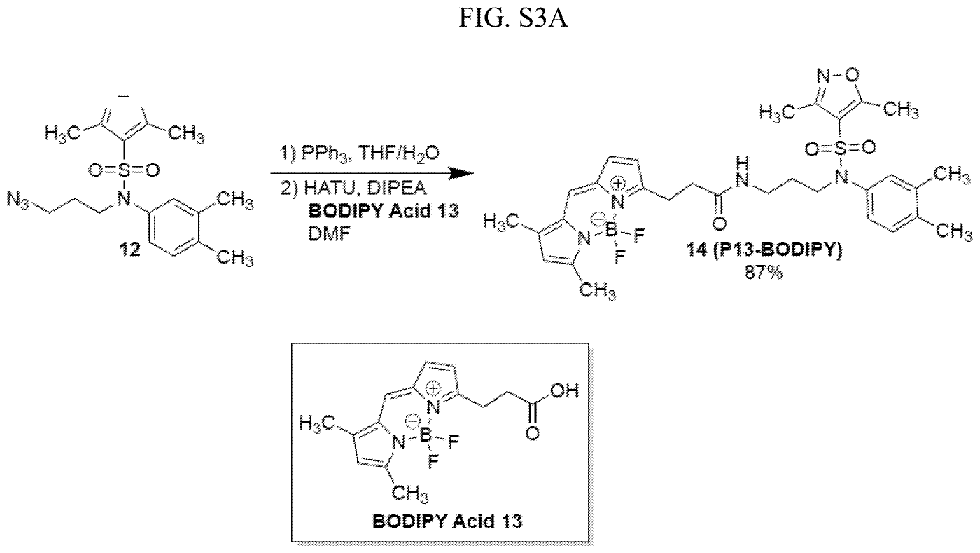

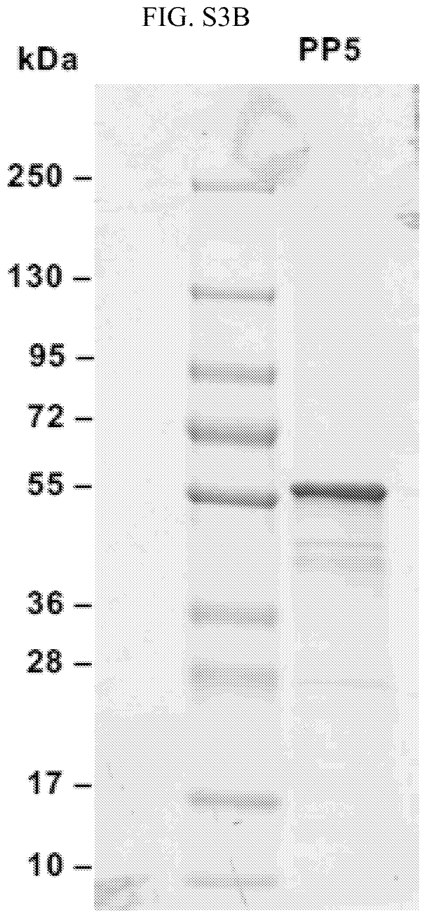

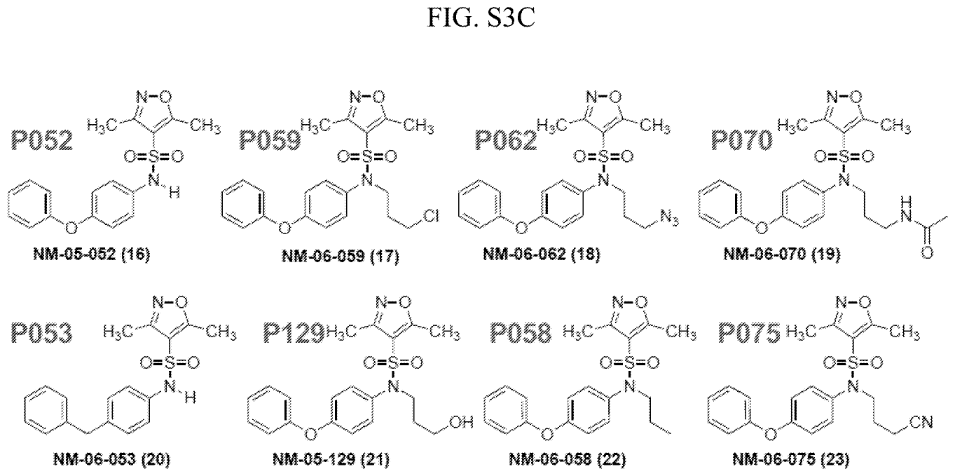

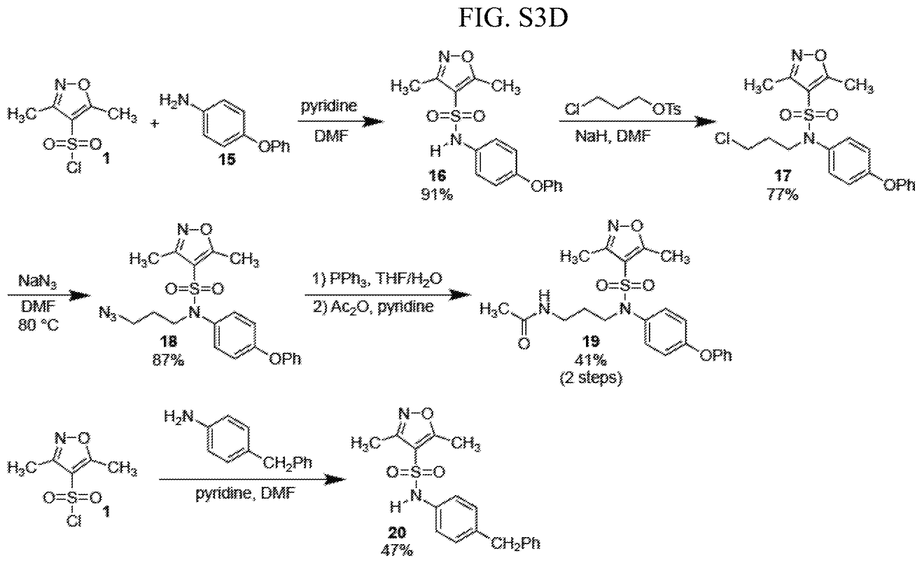

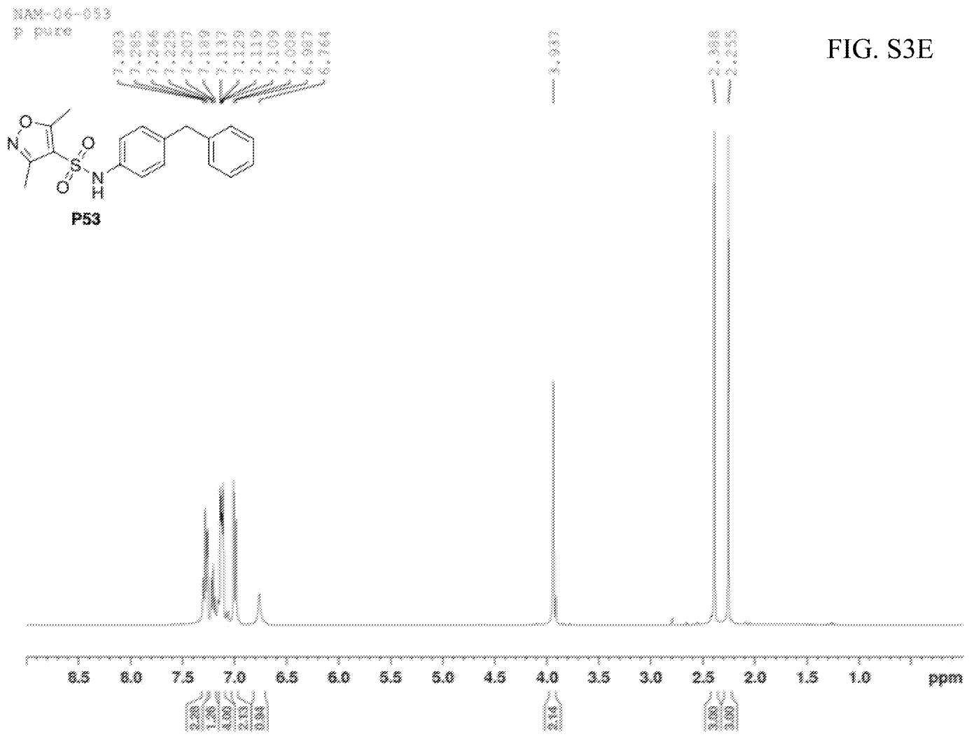

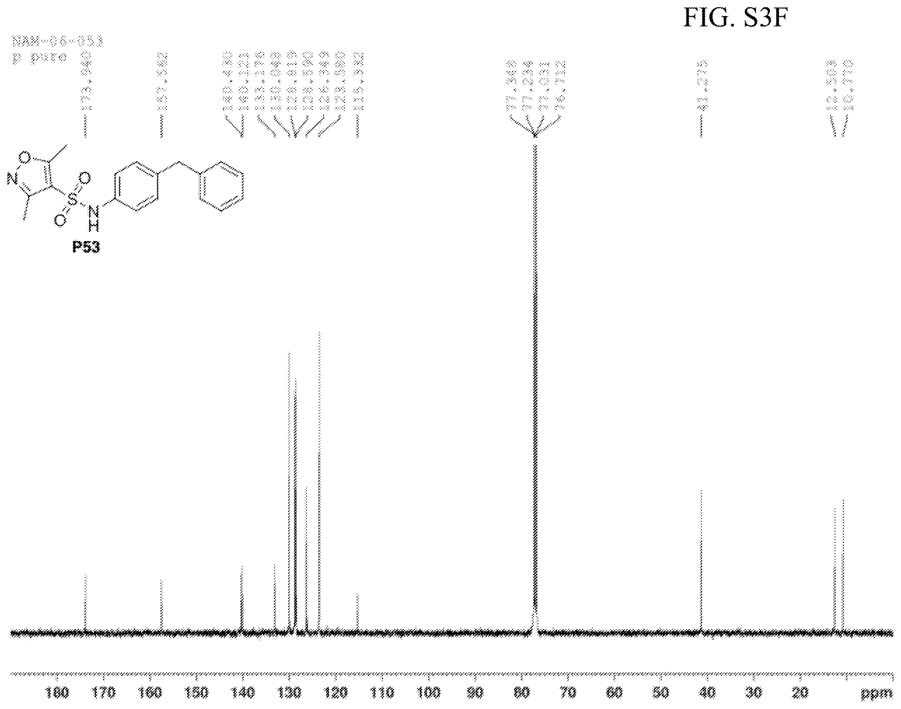

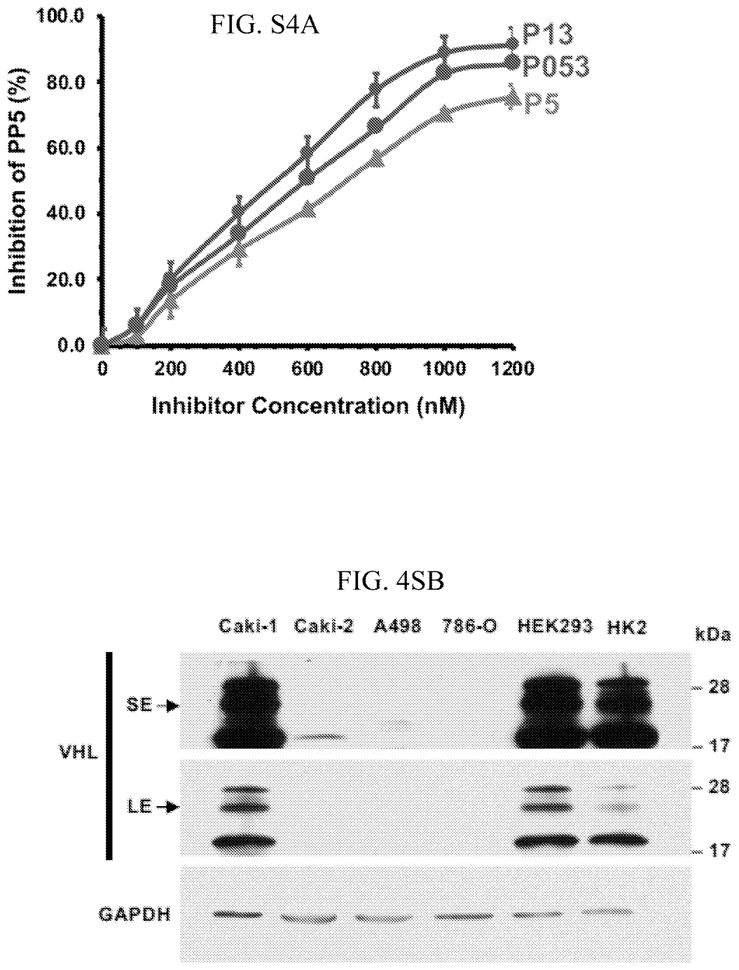

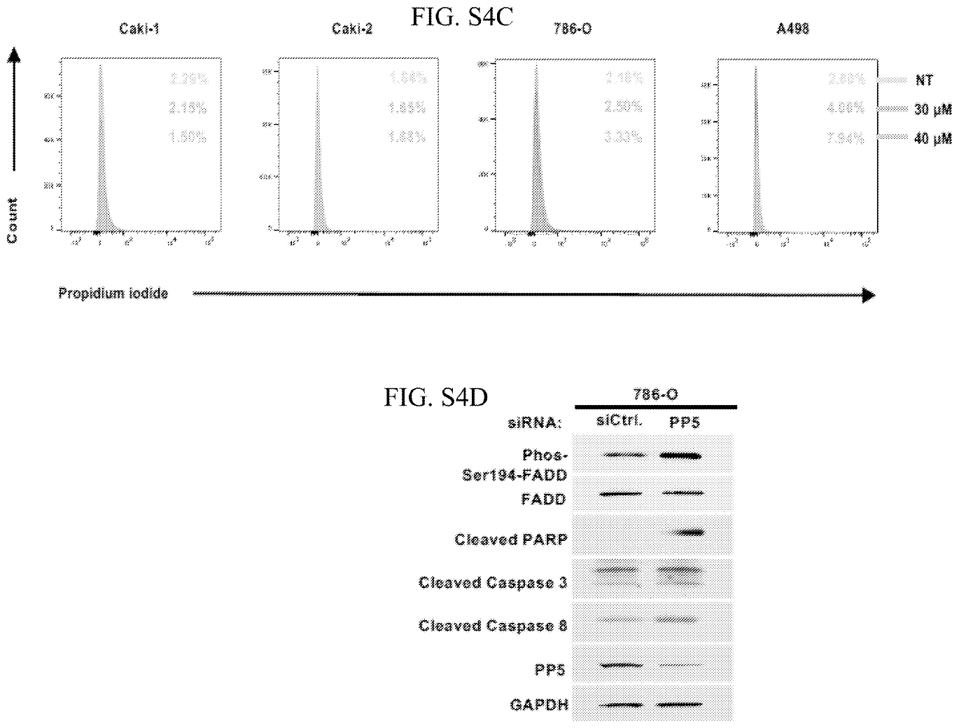

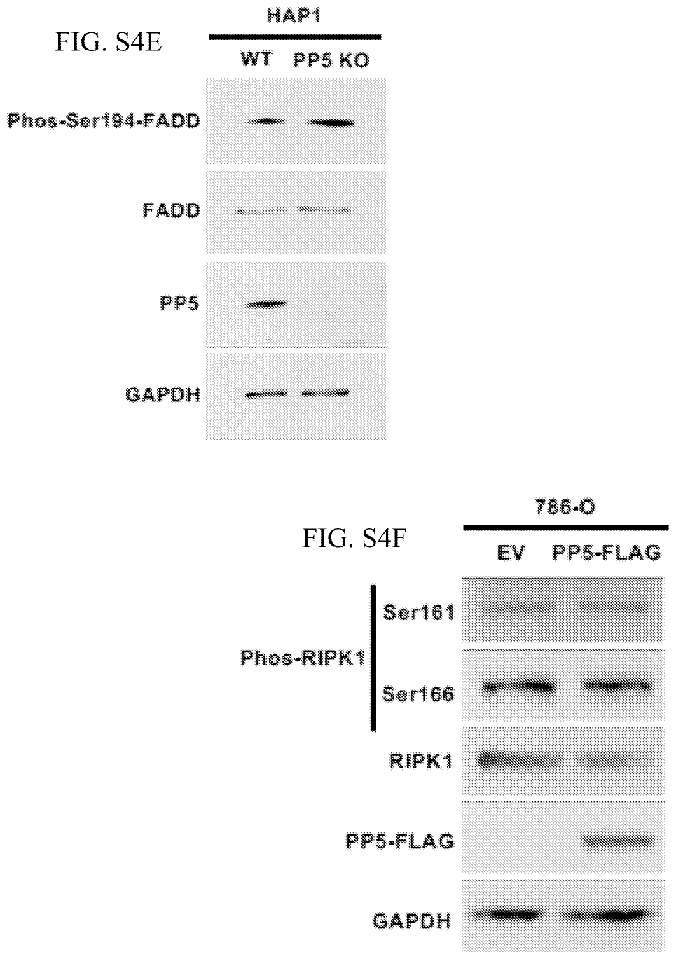

PP5 associates with intact Complex II: To gain further understanding of the dynamic of PP5 interaction with complex II, we used CRISPR/Cas9 mediated-knockout (KO) of PP5, FADD and RIPK1 in HAP1 cells. These haploid cell lines are a great resource for gene deletion in mammalian cells³⁶. Immunoprecipitation (IP) of FADD from PP5 KO cells showed FADD interaction with RIPK1 was abrogated (FIG. 4A). Similarly, IP of RIPK1 from these cells also demonstrated loss of interaction with FADD (FIG. 4A), therefore indicating that PP5 is necessary for mediating FADD:RIPK1 complex formation. We then confirmed this model in both FADD and RIPK1 KO HAP1 cells. Our data demonstrated interaction of PP5 and FADD was unaffected in RIPK1 KO cells (FIG. 4B). Likewise, interaction of PP5 and RIPK1 was maintained in FADD KO HAP1 cells (FIG. 4C).

We have shown that FADD and RIPK1 interaction is dependent on their death domains. In order to determine whether the death domain (DD) is involved in complex formation with PP5 in a cellular context we created truncated FADD (FADD-ΔDD) (FIG. 4D) and RIPK1 (RIPK1-ΔDD) (FIG. 4E) constructs that lack this death domain. PP5 failed only to co-IP with death domain-deleted FADD (FADD-ΔDD) (FIG. 4D), demonstrating the requirement of this interaction domain for complex II assembly with PP5.

Since a vast majority of PP5 substrates are also clients of the molecular chaperone Hsp90, we asked whether RIPK1 and FADD are clients of this chaperone. We therefore performed a time course assay using the Hsp90 inhibitor SNX-2112 (2 μM) in HEK293 cells. Upon inhibition of Hsp90 clients such as Tsc2, Akt and phos-Ser473-Akt were destabilized and degraded (FIG. 4F). Consistent with previously published work, we observed decreased RIPK1 protein levels after 16 hr treatment with SNX-2112 (FIG. 4F). Notably, we did not observe a decrease in FADD levels throughout the time course (FIG. 4F). This data suggests that RIPK1, but not FADD, is an Hsp90 client. To further understand the relationship between FADD and Hsp90, we immunoprecipitated endogenous FADD from 786-O cells and examined the co-IP of Hsp90 (FIG. 4G). Consistent with out previous results, we saw FADD interaction with PP5 however, we did not observe any interaction between FADD and Hsp90 (FIG. 4G). This data suggests that FADD and PP5 interact independently of Hsp90. Taken together, our data demonstrates PP5 functions to maintain complex II integrity by interacting with FADD and RIPK1 (FIG. 4H).

Discussion: PP5 plays a significant role in proliferation and survival of multiple cancers including kidney cancer. Our previous work has shown that downregulation of PP5 or pharmacologic inhibition of its regulator CK1δ caused induction of apoptosis. Here we dissected the induction of this pathway and demonstrated the direct involvement of PP5 in the extrinsic apoptotic pathway. Presence of functional PP5 appears to be important for interaction of FADD and RIPK1. Phosphorylation of S194-FADD has been shown previously to be important for its pro-apoptotic activity and suppresses tumorigenesis. Our data suggest that PP5 mediates dephosphorylation of S194-FADD, independent of Hsp90 and facilitates FADD binding to RIPK1 via their death domains. The presence of active PP5 in complex II appears to maintain suppression of extrinsic apoptosis in VHL-null ccRCC and blocks the cleavage of caspase 8. Downregulation or inhibition of PP5 leads to increased phosphorylation of S194-FADD as well as cleavage of caspase 8 and induction of the extrinsic apoptotic pathway in VHL-null ccRCC. Of note, it has also been shown that many ccRCCs exhibit lower levels of FADD than adjacent normal kidney, however, it is not yet clear whether this may be related to PP5 upregulation and how FADD is functioning in that context. Furthermore, S194-FADD phosphorylation has also been shown to regulate FADD function in cell cycle regulation. PP5 plays a number of roles in cell cycle regulation and this may be in part through FADD.

There are currently no reports of compounds that specifically inhibit PP5 function within cells. Zhang et al. designed a bifunctional molecule phosphatase recruiting chimera (PHORC) to specifically activate PP5 phosphatase activity toward the substrate Ask1. As PP5 is upregulated in a wide variety of cancers, a similar approach such as using Proteolysis Targeting Chimeras (PROTACs) to specifically degrade PP5 may be beneficial for cancer therapy. Interestingly, LB-100, which has been developed as a specific PP2A inhibitor and is now in both phase 1 and phase 2 clinical trials for various cancers, has also been shown to inhibit PP5 and PP1. There are numerous reports highlighting the role of PP5 in cancer progression and survival due to its functions in cell cycle regulation, DNA damage response, and signaling pathways. These observations suggest a therapeutic benefit of PP5 inhibitors for the clinical treatment of a wide variety cancers. Furthermore, the detailed molecular mechanism of the prosurvival role of PP5 in renal cancer prompted us to screen for and identify a small molecule inhibitor for this phosphatase. We took advantage of available X-ray crystal structures of the PP5 active site and performed threefold docking due to the promiscuity of PP5 to the metal ion in the active site, which can be a Zn, Mn or an Fe ion. We chose residues D271, N303, H304, M309, and W386 to define the “active site” for docking, as these residues are near the metal ions in the active site of PP5. Our in silico screen and further cell-based assays led us to the identification of two compounds, P5 and P13. We further developed and synthesized the P053 compound based on P13. These inhibitors have high affinity (nM range) towards PP5 and appear to make contact with the active-site residues H304, M309, W386 and R400 within the substrate-binding pocket. We also found these compounds can bind to PP5 from VHL-null ccRCC and cause apoptosis in these cells. Interestingly, although the PP5 active-site residues are conserved in other phosphatases such as PP2A, we did not observe any binding of our compounds to PP2AC, which is the catalytic subunit of PP2A, from cell lysate at the concentration at which they bound PP5.

The role of PP5 in cancer cell proliferation and survival as well as its unique structure make it an attractive therapeutic target. Important next steps for further evaluation of these PP5 inhibitors are to examine their pharmacokinetic and pharmacodynamic parameters as well as their effect on VHL-null ccRCC xenograft models. Examination in other cancers in which PP5 has been seen to play a pro-tumorigenic role is also warranted. Further refinement may be needed to optimize their on-target effects, but ultimately this work reveals a potential for small molecule PP5 inhibition in the clinic.

Limitations of the study: The PP5 inhibitors designed in this study bind specifically to and inhibit PP5 in vitro at nM range. However, in the cellular context, we used these PP5 inhibitors at low μM in order to achieve apoptosis in ccRCC cell lines. Although we do not currently have experimental evidence, there are a number of reasons that can potentially explain this phenomenon. For instance, these drugs must cross the plasma membrane to enter the cell and inhibit the target, and therefore higher amounts are needed to achieve a potent inhibitory effect. Additionally, the PP5 protein used in our biochemical analysis was expressed and purified from bacteria, and therefore it is devoid of any posttranslational modifications. However, PP5 targeted in ccRCC cells is subjected to various posttranslational modifications and this may potentially impact the binding affinity to the small molecule inhibitors of PP5.

Significance: Protein phosphatase 5 is a serine/threonine phosphatase and a co-chaperone of Hsp90 that helps regulate an array of cellular functions including stress response, proliferation, apoptosis, and DNA repair³. PP5 plays a significant role in survival and propagation of multiple cancers, which makes it a promising target for cancer therapy. Though there are several naturally occurring phosphatase inhibitors, none are specific for PP5. Additionally, the detailed molecular mechanism of PP5 prosurvival role in cancer has remained elusive. In this manuscript we have addressed these two overarching gaps in our knowledge. We previously solved the X-ray crystal structure of PP5 bound to its substrate peptide Cdc37⁶. We used this information as well as other X-ray crystal structures of PP5 to conduct an in silico drug screen. This led to identification and development of a selective and competitive inhibitors of PP5. To our knowledge, this is the first known compound that specifically targets only the PP5 phosphatase. The second part of this story focuses on dissecting the molecular mechanism of PP5 in cancer cell survival. We previously reported the prosurvival role of PP5 in kidney cancer 2. In this study we provide a mechanistic understanding of PP5 role in cells. We demonstrated that PP5 interacts with FADD, RIPK1 and caspase 8, components of the extrinsic apoptotic pathway complex II. Specifically, PP5 dephosphorylates and inactivates the death effector protein FADD in an Hsp90 independent manner, therefore preserving complex II integrity and regulating extrinsic apoptosis. Small molecule inhibition of PP5 activates this pathway, presenting a viable therapeutic strategy for renal cancer.

Experimental Model And Subject Details Cell lines: Cultured human embryonic kidney (HEK293) and human kidney 2 (HK2) cells were grown in Dulbecco's Modified Eagle Medium (DMEM, Sigma-Aldrich), 786-O cells in Roswell Park Memorial Institute (RPMI)1640 Medium (Sigma-Aldrich), A498 cells in Minimum Essential Medium (MEM, Sigma-Aldrich), Caki-1 and Caki-2 cells in McCoy's 5A Medium (Sigma-Aldrich) and wild-type (WT) HAP1 and knock-out (KO) cells in Isocove's Modified Dulbecco's Medium (IMDM, Gibco), all supplemented with 10% fetal bovine serum (FBS, Sigma-Aldrich). WT-HAP1 and KO cell lines were acquired from Horizon Discovery. All other cell lines were obtained from (American Type Culture Collection, ATCC). Cells were maintained in a CellQ incubator (Panasonic Healthcare) at 37° C. in an atmosphere containing 5% CO₂.

Plasmids: For mammalian expression, pcDNA3-PP5-FLAG and the M309C and W386F point mutations were created previously. Site-directed mutagenesis was performed to mutate R275A, H304Q, R400A, and Y451 F residues (see Table S1) and confirmed by DNA sequencing. The pcDNA3-FLAG-FADD and pcDNA3-HA-RIPK1 were purchased from Addgene. FLAG-FADD-ADD as well as HA-RIPK1-ADD constructs were subcloned using the primers listed in Table S1. For bacterial expression, we used our previously reported human PP5 gene in pGEX6P1 plasmid with an N-terminal GST tag and C-terminal His₆ tag.

Method Details Cell Transfection and Treatment: Cultured cells were split and then transfected the following day when about 40% confluent with each construct using Mirus TranslT-2020 (MirusBio) according to manufacturer's protocol. Cells were incubated at 37° C. and then extracted or collected for analysis after 24 hrs (HEK293) or 72 hrs (ccRCC cell lines). Short interfering RNA (siRNA) scramble control and PPP5C (PP5) targeting duplexes were purchased from OriGene (SKU: SR321403A, SR321403B, and SR321403C). Indicated cells were transiently transfected with the siRNA using Mirus TranslT-2020. For PP5 knock-down, either 30 nM of control siRNA or 10 nM of each PP5 siRNA duplex (A, B and C) were mixed prior to transfection. Cells were incubated at 37° C. for 72 hrs, then harvested for protein extraction. To inhibit PP5 activity, 786-O cells were incubated with indicated amount of IC261 (Abcam) for 16 hrs. Blockage of caspase activity was performed by treatment with 10 μM of z-VAD-fmk (Enzo Life Sciences) for 1 hr followed by IC261 treatment with the indicated amount for 16 hrs. Cells were then harvested for protein extraction.

Protein Extraction, Immunoprecipitation and Immunoblotting: Protein extraction from mammalian cells was carried out using methods previously described. Cell lysates were quantified using 1× Bradford reagent (Biorad). For immunoprecipitation, cell lysates were incubated with anti-FLAG antibody conjugated beads (Sigma) or anti-HA conjugated beads (ThermoFisher Scientific) at 4° C. for 2 hrs. Endogenous IPs were achieved by incubating lysate with anti-PP5 antibody (Cell Signaling), anti-FADD antibody (Cell Signaling), or anti-RIPK1 antibody (Cell Signaling) overnight followed by protein G agarose (Invitrogen) at 4° C. for 2 hrs. Immunopellets were washed 4 times with fresh lysis buffer (20 mM Tris-HCl (pH 7.4), 100 mM NaCl, 1 mM MgCl₂, 0.1% NP40, protease inhibitor cocktail (Roche), and PhosSTOP (Roche)) and eluted in 5× Laemmli buffer. Precipitated proteins were separated by SDS-PAGE and transferred to nitrocellulose membranes. Co-immunoprecipitated proteins or proteins from cell lysate were detected with antibodies recognizing FLAG, 6×-His (ThermoFisher Scientific), GAPDH (ENZO Life Sciences), Cdc37 (StressMarq), GR, phospho-GR (S211), PP5, caspase-3, cleaved caspase-3, caspase-8, cleaved caspase-8, cleaved caspase-7, cleaved-PARP, phospho-RIPK1 (S166), phospho-FADD (S194), PP2A-C, VHL (Cell Signaling), phospho-RIPK1 (S161) (Invitrogen), PP5 and phospho-Cdc37 (S13) (Abcam). Secondary antibodies raised against mouse and rabbit (Cell Signaling) were used (See Key Resources Table).

Bacterial Expression and Protein Purification of PP5: Human PPP5c was cloned into pGEX6P1 with an N-terminal GST tag and C-terminal His₆ tag. Transformed cells were grown at 37° C. in LB with 100 mg/L ampicillin until OD₆₀₀=0.6 and induced with 1 mM IPTG. Cells were harvested by centrifugation and lysed by sonication in lysis buffer (50 mM Hepes (pH 8.0), 150 mM NaCl, 0.5 mM TCEP and EDTA-free protease inhibitor cocktail tablet (Roche)). Lysate was incubated with talon resin (Takara Bio) for 1 hr at 4° C. The resin was washed three times with lysis buffer and PP5 was eluted with lysis buffer containing 250 mM imidazole. Precision protease was added to the elution overnight to cleave the GST tag and the sample was then mixed with Glutathione Sepharose resin (Cytiva) to remove the free GST and un-cleaved protein. The sample was applied to a Superdex S75 16/60 size exclusion column (GE Healthcare) and eluted in 100 mM NaCl, 20 mM Hepes pH 8, 02 mM TCEP.

Cell Viability Assay: Renal cancer cell lines 786-O, Caki-2, Caki-1, and A498 as well as the normal renal cell line HK-2 were plated at 10,000 cells per well in 96-well plates. Cells were treated with different amounts of inhibitors (P075, P059, P058, P13, P053) and DMSO was used as control (0 μM). After 24 or 48 hrs, cell viability assay was performed using the Quick Cell Proliferation Kit Plus (BioVision) according to the manufacturer's protocol. The absorbance at 450 nm was measured on a Tecan Infinite M200 Pro and proliferation rate was calculated.

In silico Docking: Virtual high throughput docking simulations were carried out using Dockblaster with the Zinc library of drug like compounds (˜3.7 million compounds). PDB structures of PP5 (PDB:3H60 and 3H66) were used as receptor structures. In preparation for docking the water molecules were eliminated, and any missing hydrogens and charges were added to the system to generate the receptor input file. The active site residues of D271, N303, H304, M309 and W386 were chosen to define the active site for docking, as these residues are nearby the active site. Once biological activity was of an inhibitor was confirmed, docking was performed again using Autodock Vina (⁵⁷ to generate docking poses which were used to guide synthetic efforts. The Autodock docking calculation was carried out using a grid per map with 40×40×40 Å points of (PDB: 3H60) in addition to a grid-point spacing of 0.375 Å, which was centered on the metals in the active site.

Synthesis of Small Molecules: General experimental information for the synthesis of small molecules: All anhydrous reactions were run under a positive pressure of argon. Dichloromethane (DCM) was dried by passage through an alumina column. 1,2-Dichloroethane (DCE) was freshly distilled from calcium hydride before use. Tetrahydrofuran (THF) was freshly distilled from Na/benzophenone still before use. DMF was distilled from calcium hydride under reduced pressure. Ethyl acetate (EA) and hexanes were purchased from commercial sources and used as received. Silica gel column chromatography was performed using 60 Å silica gel (230-400 mesh). Melting points were obtained on crystalline compounds and are uncorrected. The BODIPY acid 13 was prepared as reported previously.

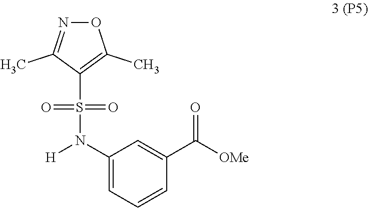 [SMILES: COC(=O)c1cccc(NS(=O)(=O)c2c(C)noc2C)c1]

Methyl 3-aminobenzoate 2 (4.23 g, 28.1 mmol) was dissolved in 52 mL DMF and cooled to 0° C. Pyridine (4.1 mL, 51 mmol) was then added and the mixture stirred for 10 minutes. 3,5-Dimethyl-isoxazole-4-sulfonyl chloride 1 (5.00 g, 25.5 mmol) was then added in portions over one hour. The reaction was then allowed to warm to room temperature (RT) and stirred for 16 hrs. The reaction was quenched by adding 1 M HCl until the pH remained below 2 (˜60 mL). The reaction mixture was then taken up in EA (400 mL) and washed with 1 M HCl (2×200 mL) and brine (2×200 mL). The organic layer was dried (MgSO₄) and concentrated to give a yellow solid. Purification using silica gel chromatography (50% EA/50% hexanes) provided pure 3 (5.65 g, 72%) as a yellow solid.

Methyl m-(3,5-dimethyl-4-isoxazolylsulfonylamino)benzoate 3 (P5). mp=140-142° C.; TLC Rf=0.45 (50% EA/50% hexanes); IR (ATR) 3226, 2970, 2359, 2341, 1692, 1588, 1339, 1306 cm⁻¹; ¹H NMR (400 MHz, CDCl₃) δ 7.84-7.81 (m, 2H), 7.79 (bs, 1H), 7.43-7.36 (m, 2H), 3.93 (s, 3H), 2.52 (s, 3H), 2.30 (s, 3H); ¹³C NMR (100 MHz, CDCl₃) δ 174.4, 166.6, 157.6, 136.5, 131.5, 129.9, 126.8, 125.7, 122.3, 115.4, 52.8, 12.7, 10.8; HRMS (ESI+) m/z calculated (calcd) for C₁₃H₁₄N₂O₅S [M+Na]⁺: 333.0516, found: 333.0515.

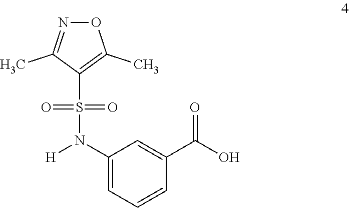 [SMILES: Cc1noc(C)c1S(=O)(=O)Nc1cccc(C(=O)O)c1]

Potassium hydroxide (8.5 g, 152 mmol) was dissolved in 40 mL of water and MeOH (160 mL) was added. The ester 3 (P5) (5.2 g, 16.9 mmol) was then added and the reaction mixture was stirred for 16 hrs at RT. The reaction mixture was then quenched by slowly adding 1M HCl until the pH remained below 2 (˜180 mL). The methanol was then removed in vacuo and the residue was taken up in water (200 mL). This mixture was extracted with EA (3×150 mL), and the combined organic extracts were dried (MgSO₄) and concentrated to provide carboxylic acid 4 (4.82 g, 96%) as an off white solid which was used without further purification.

m-(3,5-Dimethyl-4-isoxazolylsulfonylamino)benzoic acid 4. mp=184-187° C.; TLC Rf=0.51 (100% EA); IR (ATR) 3157, 2980, 2359, 1694, 1590, 1408, 1118 cm⁻¹; ¹H NMR (400 MHz, CD₃OD) δ 7.80-7.78 (m, 2H), 7.42-7.38 (m, 1H), 7.33-7.30 (m, 1H), 2.49 (s, 3H), 2.26 (s, 3H); ¹³C NMR (100 MHz, CD₃OD) δ 175.3, 168.8, 158.9, 138.6, 133.2, 130.6, 127.4, 126.7, 123.1, 116.8, 12.5, 10.7.

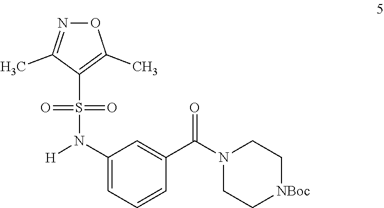 [SMILES: Cc1noc(C)c1S(=O)(=O)Nc1cccc(C(=O)N2CCN(C(=O)OC(C)(C)C)CC2)c1]

Carboxylic acid 4 (1.0 g, 3.37 mmol) was dissolved in 25 mL DMF and EDCI (0.79 g, 5.05 mmol), HOBt (80%, 0.95 g, 6.31 mmol) and diisopropylethylamine (0.88 mL, 5.05 mmol) were added. After aging for 30 min, tert-butyl piperazine-1-carboxylate (0.94 g, 5.05 mmol) was then added in one portion. The reaction mixture was stirred at RT for 3 hrs, and then quenched by the addition of brine (30 mL). The mixture was then taken up in EA (100 mL) and washed with water (2×60 mL) and brine (2×60 mL). The organic layer was then dried (MgSO₄) and concentrated. Purification of the residue using silica gel chromatography (60% EA/40% hexanes) gave amide 5 as a white solid (1.62 g, 94%).

tert-Butyl 4-[m-(3,5-dimethyl-4-isoxazolylsulfonylamino)benzoyl]-1-piperazine carboxylate 5. mp=187-189° C.; TLC Rf=0.61 (80% EA/20% hexanes); IR (ATR) 3080, 2974, 2359, 2341, 1618, 1614, 1165, 1120 cm⁻¹; ¹H NMR (400 MHz, CDCl₃) δ 8.59 (bs, 1H), 7.32-7.30 (m, 2H), 7.15-7.12 (m, 2H), 3.76-3.37 (m, 8H), 2.49 (s, 3H), 2.28 (s, 3H), 1.47 (s, 9H); ¹³C NMR (100 MHz, CDCl₃) δ 173.7, 169.7, 157.6, 154.5, 137.1, 136.2, 129.5, 123.8, 123.3, 121.4, 115.6, 80.6, 47.7, 47.6, 43.5, 42.5, 28.4, 12.6, 10.8.

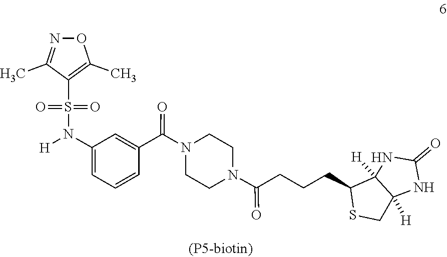 [SMILES: Cc1noc(C)c1S(=O)(=O)Nc1cccc(C(=O)N2CCN(C(=O)CCC[C@@H]3SC[C@@H]4NC(=O)N[C@@H]43)CC2)c1]

The Boc protected amine 5 (0.95 g, 20.43 mmol) was dissolved in 1:1 DCM:TFA (60 mL) and stirred for 1 hr. The solvent was then evaporated, and the crude TFA salt was used in the next step without further purification. Biotin (1.01 g, 2.19 mmol), EDCI (0.51 g, 3.3 mmol), HOBt (0.62 g, 4.1 mmol) were dissolved in 20 mL of DMF and diisopropylethylamine (0.77 mL, 4.4 mmol) was then added. After stirring for 10 min the crude TFA salt (0.80 g) was then added to the reaction mixture in portions. The reaction was then stirred for 16 hrs at RT. The reaction mixture was then taken up in brine (30 mL) and extracted with EA (3×30 mL). The combined organic layers were washed with water (2×30 mL) and brine (2×30 mL), dried (MgSO₄) and concentrated. Purification using silica gel chromatography (5% MeOH/95% DCM) gave the biotin-P5 6 as a white solid (0.53 g, 40%).

N-[3-(4-(4-[(3aS,4S,6aR)-2-Oxo-hexahydro-1H-thieno[3,4-d]imidazol-4-yl]butanoyl) piperazine-1-carbonyl)phenyl]-3,5-dimethyl-1,2-oxazole-4-sulfonamide 6 (biotin-P5). TLC Rf=0.40 (30% EA/70% toluene); IR (ATR) 3083, 2978, 2359, 2341, 1686, 1614, 1406, 1120 cm⁻¹; ¹H NMR (400 MHz, CDCl₃) δ 7.43 (t, J=7.9 Hz, 1H), 7.27-7.24 (m, 2H), 7.19-7.18 (m, 1H), 4.49 (dd, J=7.8, 4.7 Hz, 1H), 4.31 (dd, J=7.8, 4.4 Hz, 1H), 3.67-3.37 (m, 8H), 3.24-3.20 (m, 3H), 2.93 (dd, J=12.7, 4.9 Hz, 1H), 2.70 (d, J=12.7 Hz, 1H), 2.48-2.47 (m, 5H), 2.25 (s, 3H), 1.78-1.57 (m, 4H), 1.51-1.46 (m, 2H); ¹³C NMR (100 MHz, CDCl₃) δ 175.2, 174.2, 171.5, 166.0, 158.9, 138.7, 137.7, 131.0, 125.0, 124.5, 116.9, 63.3, 61.6, 57.0, 46.4, 46.3, 42.8, 42.6, 41.0, 33.6, 29.8, 29.5, 26.2, 12.6, 10.8; HRMS (ESI+) m/z calcd for C₂₆H₃₄N₆O₆S₂ [M+Na]⁺: 613.1879, found: 613.1872.

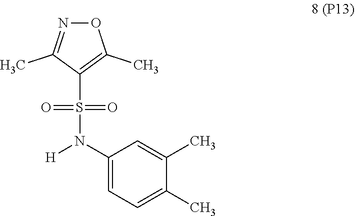

3,4-Dimethylaniline 7 (1.36 g, 11.2 mmol) was dissolved in 20 mL DMF and cooled to 0° C. Pyridine (1.7 mL, 20.4 mmol) was then added and the mixture stirred for 10 min. 3,5-Dimethyl-isoxazole-4-sulfonyl chloride 1 (2.00 g, 10.2 mmol) was then added in portions over one hour. The reaction was then allowed to warm to RT and stirred for 16 hrs. The reaction was quenched by adding 1 M HCl until the pH remained below 2 (˜25 mL). The reaction mixture was then taken up in EA (80 mL) and washed with 1 M HCl (2×50 mL) and brine (2×50 mL). The organic layer was then dried with MgSO₄. Filtration and evaporation of solvent yielded a red-brown solid which was further purified using silica gel chromatography (40% EA/60% hexanes) to provide sulfonamide 8 as a beige solid (2.04 g, 73%).

(3,5-Dimethyl-4-isoxazolylsulfonyl)(3,4-xylyl)amine 8 (P13). mp=90-92° C.; TLC Rf=0.48 (50% EA/50% hexanes); IR (ATR) 3250, 2920, 1591, 1327, 1118 cm⁻¹; ¹H NMR (400 MHz, CDCl₃) δ 7.06-7.01 (m, 2H), 6.87-6.86 (m, 1H), 6.81 (dd, J=8.0, 2.4 Hz, 1H), 2.42 (s, 3H), 2.27 (s, 3H), 2.20 (s, 3H), 2.19 (s, 3H); ¹³C NMR (100 MHz, CDCl₃) δ 174, 157.8, 138.2, 135.3, 133, 130.6, 124.4, 120.5, 115.5, 19.8, 19.3, 12.6, 10.9. HRMS (ESI+) m/z calcd for C₁₃H₁₆N₂O₃S [M+H]⁺: 281.0954, found: 281.0955.

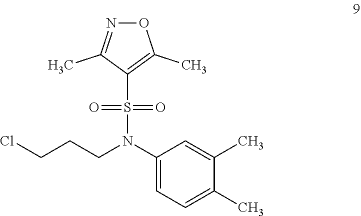 [SMILES: Cc1ccc(N(CCCCl)S(=O)(=O)c2c(C)noc2C)cc1C]

The sulfonamide 8 (P13) (1.80 g, 6.42 mmol) and K₂CO₃ (1.95 g, 14.12 mmol) were suspended in 22 mL DMF and stirred for 15 min at RT. 3-Chloropropyl p-toluenesulfonate (2.39 g, 9.63 mmol) was then added and the reaction was heated to 80° C. (oil bath temperature). After 20 hrs, the reaction mixture was allowed to cool to RT and 30 mL water was added. The reaction mixture was then extracted with EA (3×30 mL). The combined organic layers were washed with brine (100 mL), dried with MgSO₄ and filtered. After evaporating the solvent, the residue was purified using silica gel chromatography (5% EA/95% hexanes) to provide alkyl chloride 9 as a pale yellow solid (1.89 g, 83%).

(3-Chloropropyl)(3,5-dimethyl-4-isoxazolylsulfonyl)(3,4-xylyl)amine 9. mp=90-93° C.; TLC Rf=0.68 (30% EA/70% hexanes); IR (ATR) 2923, 2358, 1587, 1346, 1121, 689 cm⁻¹; ¹H NMR (400 MHz, CDCl₃) δ 7.09 (d, J=8.0 Hz, 1H), 6.94 (d, J=2.0 Hz, 1H), 6.85 (dd, J=8.4, 2.4 Hz, 1H), 3.75 (t, J=6.7 Hz, 2H), 3.57 (t, J=6.3 Hz, 2H), 2.27 (s, 3H), 2.26 (s, 3H), 2.23 (s, 3H), 1.96-1.90 (m, 2H); ¹³C NMR (100 MHz, CDCl₃) δ 173.6, 158, 138.1, 137.5, 135.5, 130.5, 129.9, 125.6, 114.8, 47.7, 41.6, 31.2, 19.8, 19.4, 12.5, 11.0.

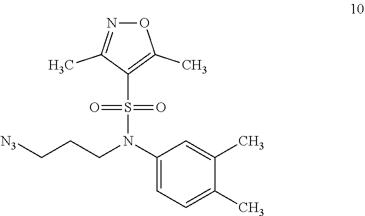 [SMILES: Cc1ccc(N(CCCN=[N+]=[N-])S(=O)(=O)c2c(C)noc2C)cc1C]

The alkyl chloride 19 (0.85 g, 2.2 mmol) was dissolved in 7 mL of DMF. Sodium azide (0.4 g, 6.6 mmol) was then added and the reaction mixture was heated to 80° C. (oil bath temperature). After 16 hrs the mixture was allowed to cool to RT and 30 mL water was added. The reaction mixture was then extracted with EA (2×50 mL). The combined the organic layers were washed with brine (100 mL), dried with MgSO₄, filtered and concentrated in vacuo. This gave azide 10 as a yellow foam (0.80 g, 92%) which was used without further purification.

(3-Azidopropyl)(3,5-dimethyl-4-isoxazolylsulfonyl)(3,4-xylyl)amine 10. TLC Rf=0.46 (30% EA/70% hexanes); IR (ATR) 2938, 2358, 2096, 1587, 1346, 1180 cm⁻¹; ¹H NMR (400 MHz, CDCl₃) δ 7.09 (d, J=8.0 Hz, 1H), 6.93 (d, J=2.1 Hz, 1H), 6.83 (dd, J=10.2, 2.2 Hz, 1H), 3.68 (t, J=6.6 Hz, 2H), 3.38 (t, J=6.6 Hz, 2H), 2.25-2.24 (m, 6H), 2.22 (s, 3H), 2.09 (s, 3H), 1.79-1.72 (m, 2H); ¹³C NMR (100 MHz, CDCl₃) δ 173.6, 158, 138.2, 137.6, 135.4, 130.5, 129.8, 125.6, 114.9, 48.4, 47.6, 27.8, 19.7, 19.4, 12.5, 11.0.

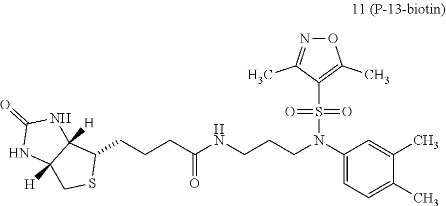

The azide 10 (1.00 g, 2.75 mmol) was dissolved in 20 mL of THF. Water (4 mL) was then added followed by triphenyl phosphine (0.79 g, 3.03 mmol). After 16 hrs at RT the solvent was evaporated and excess triphenyl phosphine was removed by passing the residue through a short plug of silica gel (20% EA/80% hexanes). This provided the amine product containing some triphenyl phosphine oxide, which was used without further purification. Biotin (1.32 g, 5.4 mmol), EDCI (0.84 g, 5.4 mmol) and diisopropylethylamine (0.95 mL, 5.4 mmol) were dissolved in 27 mL of DMF and stirred for 10 min. The crude amine (0.91 g) was then added to the reaction mixture. After 16 hrs at RT brine (30 mL) was added and the reaction mixture was extracted with EA (3×30 mL). The combined organic layers washed with water (1×30 mL) and brine (2×30 mL), dried (MgSO₄), filtered and concentrated. The residue was purified using silica gel chromatography (15% MeOH/85% DCM) to provide the amide 11 (biotin-P13) as a waxy yellow solid (0.30 g, 20%).

4-[(3aS,4S,6aR)-2-Oxo-hexahydro-1H-thieno[3,4-d]imidazol-4-yl]-N-(3-[N-(3,4-dimethylphenyl)3,5-dimethyl-1,2-oxazole-4-sulfonamido]propyl}butanamide 11 (biotin-P13). TLC Rf=0.32 (10% MeOH/90% DCM); IR (ATR) 3208, 2923, 2359, 2341, 1696, 1641, 1342, 1116, 688 cm⁻¹; ¹H NMR (400 MHz, CDCN₃) δ 7.14 (d, J=8.4 Hz, 1H), 7.00-6.99 (m, 1H), 6.92 (dd, J=8.0, 2.2 Hz, 1H), 6.47 (bs, 1H), 5.55 (bs, 1H). 5.23 (bs, 1H), 4.42-4.39 (m, 1H), 4.23-4.20 (m, 1H), 3.61 (t, J=5.1 Hz, 2H), 3.17-3.12 (m, 3H), 2.87 (dd, J=12.7, 4.9 Hz, 1H), 2.63 (d, J=12.7 Hz, 1H), 2.27 (s, 3H), 2.25 (s, 3H), 2.21 (s, 3H), 2.08 (t, J=7.4 Hz, 3H), 1.99 (s, 3H), 1.71-1.48 (m, 6H), 1.39-1.31 (m, 2H); ¹³C NMR (100 MHz, CDCN₃) δ 174.7, 173.7.9, 163.9, 158.8, 138.8, 138.3, 136.5, 131.0, 130.7, 127.0, 115.5, 62.3, 60.7, 56.3, 48.7, 41.1, 36.9, 36.3, 29, 28.9, 28.8, 26.4, 19.7, 19.4, 12.8, 11.1; HRMS (ESI+) m/z calcd for C₂₆H₃₇N₅O₅S₂ [M+Na]⁺: 586.2134, found: 586.2119.

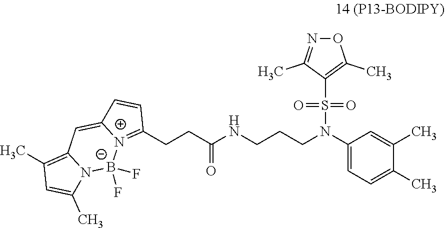

The azide 12 (1.00 g, 2.75 mmol) was dissolved in 20 mL of THF. Water (4 mL) was then added followed by triphenyl phosphine (0.79 g, 3.03 mmol). After 16 hrs at RT the solvent was evaporated and excess triphenyl phosphine was removed by passing the residue through a short plug of silica gel (20% EA/80% hexanes). This provided the amine product containing some triphenyl phosphine oxide, which was used without further purification. 4,4-Difluoro-5,7-dimethyl-4-bora-3a,4a-diaza-s-indacene-3-propionic acid (BODIPY Acid 13) (0.043 g, 0.148 mmol) was dissolved in DMF (2 mL) and HATU (0.112 g, 0.296 mmol) was added. After 5 min the amine from the previous step (0.050 g, 0.148 mmol) was added followed by addition of diisopropylethylamine (0.05 mL, 0.296 mmol). The reaction mixture was stirred at RT for 16 hrs, and was then quenched by addition of sat. NH₄Cl (10 mL) and then extracted with DCM (3×5 mL). The combined organic layers washed with water (1×5 mL) and brine (2×5 mL), dried (MgSO₄), filtered and concentrated. Purification of the residue by column chromatography (30% DCM/EA) yielded the amide 14 (BODIPY-P13) as dark red crystals (0.078 g, 87%).

12-[2-((3-[N-(3,4-Dimethylphenyl)3,5-dimethyl-1,2-oxazole-4-sulfonamido]propyl}carbamoyl)ethyl]-2,2-difluoro-4,6-dimethyl-1λ⁵,3-diaza-2-boratricyclo[7.3.0.0^{3,7}]dodeca 1(12), 4,6,8,10-pentaen-1-ylium-2-uide 14 (BODIPY-P13). mp=175-177° C.; TLC Rf=0.68 (30% DCM/EA); ¹H NMR (400 MHz, CDCl₃) δ 7.08 (s, 1H), 7.06 (s, 1H), 6.91 (s, 1H), 6.85 (d, J=3.7 Hz, 1H), 6.81 (d, J=7.9 Hz, 1H), 6.27 (d, J=3.7 Hz, 1H), 6.13 (s, 1H), 5.99 (bs, 1H), 3.57 (t, J=6.4 Hz, 2H), 3.33-3.24 (m, 4H), 2.62 (t, J=7.5 Hz, 2H), 2.56 (s, 3H), 2.26 (s, 3H), 2.24 (s, 6H), 2.22 (s, 3H), 2.06 (s, 3H), 1.61-1.54 (m, 2H); ¹³C NMR (100 MHz, CDCl₃) δ 173.4, 171.8, 160.3, 158, 157.4, 143.9, 138, 137.4, 135.3, 135.1, 133.3, 130.4, 129.9, 128.1, 125.7, 123.8, 120.4, 117.4, 115, 47.8, 36, 35.9, 27.8, 24.8, 19.8, 19.4, 14.9, 12.5, 11.3, 11.0.

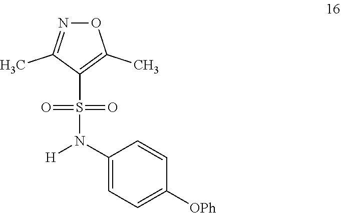 [SMILES: Cc1noc(C)c1S(=O)(=O)Nc1ccc(Oc2ccccc2)cc1]

4-Phenoxy aniline 15 (1.00 g, 5.4 mmol) was dissolved in 15 mL DMF and cooled to 0° C. Pyridine (0.90 mL, 10.8 mmol) was then added. After 10 min, 3,5-dimethyl-isoxazole-4-sulfonyl chloride 1 (1.16 g, 5.94 mmol) was added in portions over 15 min. After the addition was complete the ice bath was removed and the reaction mixture was allowed to warm to RT. After 16 hrs the reaction was quenched by adding 1M HCl until the pH remained below 2 (˜15 mL). The reaction mixture was then extracted with EA (2×20 mL). The combined organic layers were washed with 1M HCl (2×20 mL) and brine (2×20 mL), dried (MgSO4) and concentrated. The residue was purified using silica gel chromatography (40% EA/60% hexanes) that provided sulfonamide 16 as an off-white powder (1.58 g, 91%).

(3,5-Dimethyl-4-isoxazolylsulfonyl)(p-phenoxyphenyl)amine 16 (P052). mp=106-109° C.; TLC Rf=0.30 (50% EA/hexanes); ¹H NMR (400 MHz, CDCl₃) δ 7.35 (t, J=7.5 Hz, 2H), 7.14 (t, J=7.4 Hz, 1H), 7.05 (d, J=7.4, 2H), 6.98 (d, J=8.3 Hz, 2H), 6.93 (d, J=9.0 Hz, 2H), 6.44 (bs, 1H), 2.42 (s, 3H), 2.29 (s, 3H); ¹³C NMR (100 MHz, CDCl₃) δ 173.9, 157.6, 156.6, 156.5, 129.9, 129.8, 126, 123.9, 119.4, 119.2, 115.2, 12.5, 10.8; Anal. Calcd for C₁₇H₁₆N₂O₄S: C, 59.29; H, 4.68; N, 8.13. Found: C, 59.23; H, 4.78; N, 8.17.

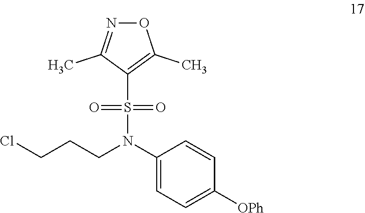 [SMILES: Cc1noc(C)c1S(=O)(=O)N(CCCCl)c1ccc(Oc2ccccc2)cc1]

The sulfonamide 16 (0.2 g, 0.58 mmol) and K₂CO₃ (0.24 g, 1.74 mmol) were suspended in 25 mL of MeCN and stirred for 15 min at rt. 3-Chloropropyl p-toluenesulfonate (0.29 g, 1.16 mmol (prepared as described in ⁵⁹) was then added and the reaction mixture warmed to reflux. After 20 hrs the reaction was allowed to cool to RT and 30 mL water was added. The reaction mixture was the extracted with EA (3× is 30 mL). The combined organic extracts were washed with brine (100 mL), dried (MgSO₄), filtered and concentrated. Purification of the residue using silica gel chromatography (50% EA/50% hexanes) gave alkyl chloride 17 as an off-white powder (0.190 g, 77%).

(3-Chloropropyl)(3,5-dimethyl-4-isoxazolylsulfonyl)(p-phenoxyphenyl)amine 17 (P059). mp=95-98° C.; TLC Rf=0.21 (50% DCM/hexanes); ¹H NMR (400 MHz, CDCl₃) δ 7.38 (t, J=7.8 Hz, 2H), 7.17 (t, J=7.4 Hz, 1H), 7.11 (d, J=8.8 Hz, 2H), 7.01 (d, J=7.8 Hz, 2H), 6.96 (d, J=8.8 Hz, 1H), 3.77 (t, J=6.8 Hz, 2H), 3.59 (t, J=6.3 Hz, 2H), 2.32 (s, 3H), 2.15 (s, 3H), 2.00-1.94 (m, 2H); ¹³C NMR (100 MHz, CDCl₃) δ 173.7, 157.9, 156, 132.5, 130.2, 130, 124.3, 119.5, 118.9, 114.7, 47.9, 41.5, 31.3, 12.5, 11.0; Anal. Calcd for C₂₀H₂₁ClN₂O₄S: C, 57.07; H, 5.03; N, 6.66. Found: C, 56.98; H, 5.10; N, 6.60.

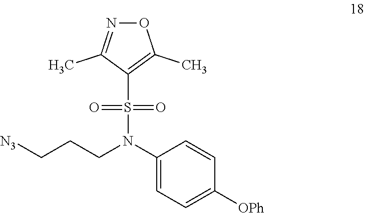 [SMILES: Cc1noc(C)c1S(=O)(=O)N(CCCN=[N+]=[N-])c1ccc(Oc2ccccc2)cc1]

The alkyl chloride 17 (0.100 g, 0.24 mmol) was dissolved in 1 mL of DMF. Sodium azide (0.050 g, 0.71 mmol) was then added and the reaction mixture was warmed to 80° C. After 16 hrs the reaction mixture was allowed to cool to RT and 10 mL water was added. The reaction mixture was extracted with EA (3×5 mL). The combined organic extracts were washed with brine (10 mL), dried (MgSO₄), filtered and concentrated. This provided the azide 18 a tan solid (0.090 g, 87%).

(3-Azidopropyl)(3,5-dimethyl-4-isoxazolylsulfonyl)(p-phenoxyphenyl)amine 18 (P062). mp=72-76° C.; TLC Rf=0.63 (30% EA/70% hexanes); IR (ATR) 3076, 2935, 2091, 1585, 1484, 1345 cm⁻¹; ¹H NMR (400 MHz, CDCl₃) δ 7.37 (t, J=7.7 Hz, 2H), 7.17 (t, J=7.4 Hz, 1H), 7.11 (d, J=8.8 Hz, 2H), 7.01 (d, J=8.2 Hz, 2H), 6.97 (d, J=8.7 Hz, 2H), 3.71 (t, J=6.7 Hz, 2H), 3.34 (t, J=6.6 Hz, 2H), 2.31 (s, 3H), 2.15 (s, 3H), 1.79-1.72 (m, 2H); ¹³C NMR (100 MHz, CDCl₃) δ 173.6, 157.9, 157.8, 156, 132.4, 130.2, 130, 124.3, 119.5, 118.9, 114.7, 48.4, 47.8, 27.9, 12.5, 11.0; Anal. Calcd for C₂₀H₂₁N₅O₄S: C, 56.19; H, 4.95; N, 16.38. Found: C, 56.15; H, 4.87; N, 16.45.

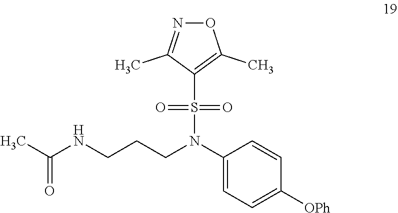 [SMILES: CC(=O)NCCCN(c1ccc(Oc2ccccc2)cc1)S(=O)(=O)c1c(C)noc1C]

The azide 18 (0.55 g, 1.29 mmol) was dissolved in 10 mL of THF and water (2 mL) was added followed by triphenyl phosphine (0.38 g, 1.42 mmol). After 16 hrs at RT the solvent was evaporated and the residue was put through a plug of silica gel (20% EA/80% hexanes). This gave the amine product containing small amounts of triphenyl phosphine oxide. This amine (0.05 g, 0.12 mmol) was dissolved in 0.5 mL of DCM and cooled to 0° C. Pyridine (0.06 mL, 0.07 mmol) was then added, followed by acetic anhydride (0.013 mL, 0.14 mmol). After 3 hrs at RT 5 mL water was added and the reaction mixture was extracted with DCM (3×5 mL). The combined organic extracts were washed with sat. aq. NaHCO₃ (5 mL) and brine (5 mL), dried (MgSO₄), filtered and concentrated. Purification of the residue using silica gel chromatography (10% MeOH/DCM) provided acetamide 19 as a white foam (0.040 g, 73%).

1-(3-[(3,5-Dimethyl-4-isoxazolylsulfonyl)(p-phenoxyphenyl)amino]propylamino)-1-ethanone 19 (P070). TLC Rf=0.44 (100% EA); IR (ATR) 3265, 3097, 2936, 1629, 1502, 1343, 1246 cm⁻¹; ¹H NMR (400 MHz, CDCl₃) δ 7.36 (t, J=7.6 Hz, 2H), 7.16 (t, J=7.4 Hz, 1H), 7.36 (d, J=8.8 Hz, 2H), 6.93 (d, J=7.8 Hz, 2H), 6.95 (d, J=8.8 Hz, 1H), 3.67 (t, J=6.3 Hz, 2H), 3.36 (q, J=6.2 Hz, 2H), 2.30 (s, 3H), 2.14 (s, 3H), 1.99 (s, 3H), 1.69-1.62 (m, 2H); ¹³C NMR (100 MHz, CDCl₃) δ 173.5, 170.5, 157.9, 157.7, 156, 132, 130.3, 130, 124.3, 119.6, 118.9, 114.8, 47.9, 36, 27.5, 23.2, 12.5, 11.0; Anal. Calcd for C₂₂H₂₅N₃O₅S: C, 59.58; H, 5.68; N, 9.47. Found: C, 59.63; H, 5.74; N, 9.26.

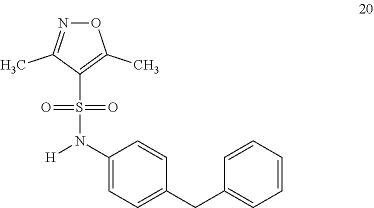 [SMILES: Cc1noc(C)c1S(=O)(=O)Nc1ccc(Cc2ccccc2)cc1]

4-Benzylaniline (0.5 g, 2.73 mmol) was dissolved in 10 mL of DMF and cooled to 0° C. Pyridine (0.44 mL, 5.46 mmol) was then added. After 10 min, 3,5-dimethyl-isoxazole-4-sulfonyl chloride 1 (0.59 g, 3 mmol) was added in portions over 15 min. After 16 hrs the reaction was quenched by adding 1 M HCl until the pH was maintained below 2. The reaction mixture was then taken up in EA (50 mL) and washed with 1 M HCl (2×20 mL) and brine (2×20 mL). The organic layer was then dried (MgSO₄), filtered and concentrated. The residue was purified using silica gel chromatography (10% EA/90% hexanes) to obtain sulfonamide 20 as a light brown foam (0.482 g, 47%).

(3,5-Dimethyl-4-isoxazolylsulfonyl)(p-benzylphenyl)amine 20 (P053). mp=111-115° C.; IR (ATR) 3227, 2951, 1588, 1490, 1337, 1179 cm⁻¹; ¹H NMR (400 MHz, CDCl₃) δ 7.28 (t, J=7.2 Hz, 2H), 7.20 (t, J=7.3 Hz, 1H), 7.14-7.11 (m, 4H), 6.99 (d, J=8.4 Hz, 2H), 6.8 (bs, 1H), 3.94 (s, 2H), 2.39 (s, 3H), 2.25 (s, 3H); ¹³C NMR (100 MHz, CDCl₃) δ 173.9, 157.6, 140.4, 140.1, 133.2, 130, 128.8, 128.6, 126.3, 123.6, 115.3, 41.3, 12.5, 10.7; Anal. Calcd for C₁₈H₁₈N₂O₃S: C, 63.14; H, 5.30; N, 8.18. Found: C, 63.19; H, 5.27; N, 7.99.

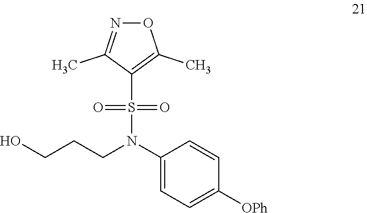 [SMILES: Cc1noc(C)c1S(=O)(=O)N(CCCO)c1ccc(Oc2ccccc2)cc1]

The sulfonamide 16 (0.2 g, 0.58 mmol) and K₂CO₃ (0.18 g, 1.28 mmol) were suspended in 2.5 mL of MeCN and stirred for 15 min at RT. 3-Chloropropanol (0.082 g, 0.087 mmol) was then added and the reaction mixture was heated to reflux. After 20 is hrs the reaction mixture was allowed to cool to RT and 30 mL water was added. The reaction mixture was extracted with EA (3×10 mL) and the combined organic layers were washed with brine (20 mL), dried (MgSO₄), filtered and concentrated. Purification of the residue using silica gel chromatography (10% EA/90% hexanes) yielded alcohol 21 as a white powder (0.09 g, 39%).

3-[(3,5-Dimethyl-4-isoxazolylsulfonyl)(p-phenoxyphenyl)amino]propanol 21 (P129). TLC Rf=0.90 (50% EA/50% hexanes); ¹H NMR (400 MHz, CDCl₃) δ 7.37 (t, J=7.6 Hz, 2H), 7.17 (t, J=7.4 Hz, 1H), 7.12 (d, J=9.1 Hz, 2H), 6.96 (d, J=8.9 Hz, 2H), 3.79-3.75 (m, 4H), 2.33 (s, 3H), 2.16 (s, 3H), 1.72-1.66 (m, 4H); ¹³C NMR (100 MHz, CDCl₃) δ 173.5, 157.8, 157.7, 156.1, 132.4, 130.4, 130, 124.2, 119.5, 118.9, 114.9, 58.8, 47.3, 30.7, 12.6, 11.1; Anal. Calcd for C₂₀H₂₂N₂O₅S: C, 59.69; H, 5.51; N, 6.96. Found: C, 59.61; H, 5.63; N, 6.79.

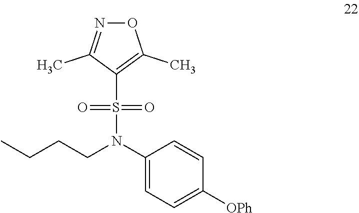 [SMILES: CCCCN(c1ccc(Oc2ccccc2)cc1)S(=O)(=O)c1c(C)noc1C]

The sulfonamide 16 (0.2 g, 6.42 mmol) and K₂CO₃ (0.24 g, 1.74 mmol) were dissolved in 20 mL of MeCN and stirred for 15 min at RT. 1-Bromobutane (0.12 mL, 1.16 mmol) was then added and the reaction was heated to 80° C. After 20 hrs, 30 mL water was added and reaction mixture was extracted with EA (3×10 mL). The combined organic layers were washed with brine (50 mL) dried (MgSO₄), filtered and concentrated. Purification of the residue with silica gel chromatography (10% EA/90% hexanes) gave sulfonamide 22 as an off-white foam (0.19 g, 83%).

N-Butyl(3,5-dimethyl-4-isoxazolylsulfonyl)(p-phenoxyphenyl)amine 22 (P058). TLC Rf=0.61 (30% EA/70% hexanes); IR (ATR) 3065, 2951, 2868, 1588, 1503, 1340, 1180 cm⁻¹; ¹H NMR (400 MHz, CDCl₃) δ 7.41 (t, J=7.6 Hz, 2H), 7.19 (t, J=7.4 Hz, 1H), 7.12 (d, J=8.9 Hz, 2H), 7.04 (d, J=7.7 Hz, 2H), 6.98 (d, J=8.9 Hz, 2H), 3.63 (t, J=6.8 Hz, 2H), 2.32 (s, 3H), 2.18 (s, 3H), 1.50-1.34 (m, 4H), 0.92 (t, J=7.2 Hz, 2H); ¹³C NMR (100 MHz, CDCl₃) δ 173.4, 157.9, 157.6, 156.2, 132.6, 130.4, 130, 124.1, 119.4, 118.8, 115.0, 50.1, 30.2, 19.5, 13.5, 12.5, 11.0; Anal. Calcd for C₂₁H₂₄N₂O₄S: C, 62.98; H, 6.04; N, 6.99. Found: C, 62.90; H, 6.14; N, 6.85.

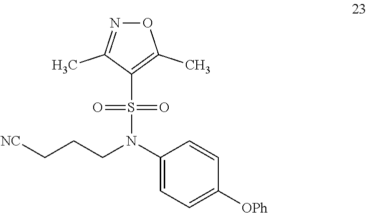 [SMILES: Cc1noc(C)c1S(=O)(=O)N(CCCC#N)c1ccc(Oc2ccccc2)cc1]

Alkyl chloride 17 (0.70 g, 1.67 mmol) was dissolved in DMF (15 mL) and NaCN (0.25 g, 5 mmol) was added. The reaction was then heated to 85° C. (oil bath temperature). After 18 hrs the reaction mixture was allowed to cool to RT and poured into water (50 mL). The resulting mixture was extracted with EA (3×30 mL). The combined organic extracts were washed with water (50 mL) and brine (2×50 mL), dried (MgSO₄), filtered and concentrated. The residue was purified using silica gel chromatography (30% EA/70% hexanes) to obtain nitrile 23 as an off-white crystalline solid (0.62 g, 91%).

4-[(3,5-Dimethyl-4-isoxazolylsulfonyl)(p-phenoxyphenyl)amino]butyronitrile 23 (P075). mp=78-83° C.; TLC Rf=0.32 (30% EA/70% hexanes); IR (ATR) 3384, 3225, 2938, 2246, 1732, 1586, 1487 cm⁻¹; ¹H NMR (400 MHz, CDCl₃) δ 7.41 (t, J=7.6 Hz, 2H), 7.20 (t, J=7.4 Hz, 1H), 7.13 (d, J=8.9 Hz, 2H), 7.04 (d, J=7.7 Hz, 2H), 6.99 (d, J=8.9 Hz, 1H), 3.76 (t, J=6.5 Hz, 2H), 2.51 (t, J=7.3 Hz, 2H), 2.33 (s, 3H), 2.16 (s, 3H), 1.93-1.87 (m, 2H); ¹³C NMR (100 MHz, CDCl₃) δ 173.8, 158.1, 157.8, 155.9, 132, 130.2, 130, 124.4, 119.7, 118.9, 118.7, 114.5, 49.2, 24.5, 14.4, 12.5, 11.0; Anal. Calcd for C₂₁H₂₁N₃O₄S: C, 61.30; H, 5.14; N, 10.21. Found: C, 61.25; H, 5.17; N, 10.06.

Binding Measurements and Anisotropy: Recombinant PP5-His₆ at the indicated concentrations was incubated on ice in 100 mM NaCl, 20 mM HEPES pH 8.0, 1% glycerol, 0.2 mM tris(2-carboxyethyl)phosphine (TCEP) and 0.5 mM MnCl2 with 10 nM BODIPY-labeled P13 in 2% DMSO for 30 min in opaque black 96 well plates (Corning). Uncalibrated fluorescence anisotropy was then measured using a SpectraMax i3 equipped with fluorescein anisotropy module (Molecular Devices). Curves were fit to a one-site binding equation using GraphPad Prism version 9.5.0. y=y₀+A*x/(K_{d}+x) where y is measured uncalibrated anisotropy, y₀ is the y intercept, A is the amplitude of the curve, x is the concentration of PP5 used, and K_{d} is the measured dissociation constant. Data are presented as mean±SEM.

PP5 Phosphatase Activity and Inhibition Assay: The phosphatase activity of the recombinant PP5-His₆ was measured using the PiPer™ Phosphate Assay Kit (Thermo Fisher Scientific) as described in the manufacturer's protocol. Standard curve with linear fit line was created from 0-1 nM Pifinal concentration reactions. 1 nM of PP5-His₆ was added to each reaction with indicated amounts of custom synthesized substrate phospho-S211-glucocorticoid receptor (PhosS211-GR) peptide (see Key Resources Table) as specific substrate (Thermo Fisher Scientific). Reactions were run in triplicate and incubated at 37° C. for over 10 min. Reaction was also performed in the presence of different amounts (100-1200 nM) of PP5 inhibitors (P5, P13 and P053). Enzyme kinetics were calculated and plotted using Lineweaver Burk plot and web-based tool (https://www.aatbio.com/tools/ic50-calculator) for calculating IC₅₀ and (https://bioinfo-abcc.ncifcrf.gov/IC50_Ki_Converter/index.php) for converting IC₅₀ to Kᵢ values for inhibitors of enzyme activity and ligand binding.

Flow Cytometric Analysis: Fluorescence-activated cell sorting (FACS) analysis was performed according to the protocol in the Annexin V:FITC kit (Bio-Rad). In brief, cells were plated in 10 cm dish at 0.5×10⁶ and incubated at 37° C. for 18 hrs. Cells were subsequently treated with compound P053 at the indicated concentrations for 18 hrs. Cells were trypsinized, collected and washed once with 1× binding buffer (included in the kit). Propidium iodide was added, then the cells were immediately run on a Becton Dickinson LSRFortessa instrument (BD Biosciences). Data were analyzed using FlowJo software version 10.7.1 for Windows (BD Biosciences).

Biotin-P5 and biotin-P13 Pulldown: HEK293 cells were transiently transfected with PP5-FLAG or active site point mutants and protein lysate extracted. Lysate was incubated with 0.01-10 μM biotin-P5 or biotin P-13 as indicated at 4° C. for 1 hr then added to streptavidin-conjugated agarose and incubated at 4° C. for 1 hr. Following three washes with fresh extraction buffer bound proteins were eluted in 5× Laemmli buffer and analyzed by Western blot. Competition experiment with P053 was conducted with protein lysate from untreated 786-O cells. Lysate was incubated with 1 μM biotin-P5 or biotin-P13 for 1 hr followed by competition with 1 μM P053 at 4° C. for 30 min This was then incubated with streptavidin-conjugated agarose at 4° C. for 1 hr prior to washing 3× with fresh extraction buffer and elution in 5× Laemmli buffer. Samples were run by SDS PAGE, transferred to nitrocellulose membrane, and analyzed by Western blot.

Quantification, statistical analysis and reproducibility: The data presented are representative of three biological replicates, unless otherwise specified. All statistics were performed using GraphPad Prism version 9.5.0 for Windows (GraphPad Software at graphpad.com). Statistical significance was ascertained between individual samples using a parametric unpaired t-test. Significance is denoted by asterisks in each figure: *P<0.05; **P<0.01; ***P<0.001; ****P<0.0001. Error bars represent the standard deviation for three independent experiments, unless otherwise indicated.

### Resources Table:

| | | |
|---|---|---|
| REAGENT or RESOURCE | SOURCE | IDENTIFIER |

Antibodies

| | | |
|---|---|---|
| Rabbit anti-FLAG tag | Thermo Scientific | Cat# PA1-984B; |
| | | RRID: AB_347227 |
| Rabbit anti-HA tag (C29F4) | Cell Signaling | Cat# 3724; RRID: AB_1549585 |
| | Technology | |
| Mouse anti-6x-His | Thermo Scientific | Cat# MA1-21315; |
| epitope tag (HIS.H8) | | RRID: AB_557403 |
| Mouse anti-GAPDH (1D4) | Enzo Life Sciences | Cat# ADI-CSA-335; |
| | | RRID: AB_10617247 |
| Rabbit anti-PP5 | Cell Signaling | Cat# 2289; RRID: AB_2168757 |
| | Technology | |
| Mouse anti-PP5 (2E12) | Abcam | Cat# ab123919; |
| | | RRID: AB_10976136 |
| Rabbit anti-FADD | Cell Signaling | Cat# 2782; RRID: AB_2100484 |
| | Technology | |
| Rabbit anti-phos-Ser194- | Cell Signaling | Cat# 2781; RRID: AB_2100485 |
| FADD | Technology | |
| Rabbit anti-RIPK1 | Cell Signaling | Cat# 3493; RRID: AB_2305314 |
| | Technology | |
| Rabbit anti-phos-Ser161- | Invitrogen | Cat# PA5-105640; |
| RIPK1 | | RRID: AB_2817068 |
| Rabbit anti-phos-Ser166- | Cell Signaling | Cat# 44590; RRID: AB_2799268 |
| RIPK1 | Technology | |
| Rabbit anti-phos-Ser13- | Abcam | Cat# ab108360; |
| Cdc37 (EPR4979) | | RRID: AB_10859480 |
| Rabbit anti-Cdc37 | StressMarq | Cat# SPC-142; |
| | Biosciences | RRID: AB_2570605 |
| Rabbit anti-GR (D6H2L) | Cell Signaling | Cat# 12041; RRID: AB_2631286 |
| | Technology | |
| Mouse anti-GR (D4X9S) | Cell Signaling | Cat# 47411; RRID: AB_2799324 |
| | Technology | |
| Rabbit anti-phospho-GR S211 | Cell Signaling | Cat# 4161; RRID: AB_2155797 |
| | Technology | |
| Rabbit anti-cleaved-PARP | Cell Signaling | Cat# 5625; RRID: AB_10699459 |
| | Technology | |
| Rabbit anti-caspase-3 | Cell Signaling | Cat# 9665; RRID: AB_2069872 |
| | Technology | |
| Rabbit anti-cleaved caspase-3 | Cell Signaling | Cat# 9664; RRID: AB_2070042 |
| | Technology | |
| Rabbit anti-cleaved caspase-7 | Cell Signaling | Cat# 9491; RRID: AB_2068144 |
| | Technology | |
| Mouse anti-caspase-9 | Cell Signaling | Cat# 9508; RRID: AB_2068620 |
| | Technology | |
| Mouse anti-caspase-8 | Cell Signaling | Cat# 9746; RRID: AB_2275120 |
| | Technology | |
| Rabbit anti-cleaved | Cell Signaling | Cat# 9496; RRID: AB_561381 |
| caspase-8 | Technology | |
| Rabbit anti-PP2A C Subunit | Cell Signaling | Cat# 2038; RRID: AB_2169495 |
| | Technology | |
| Rabbit anti-VHL | Cell Signaling | Cat# 68547; RRID: AB_2716279 |
| | Technology | |
| Rabbit anti-phospho-Akt S473 | Cell Signaling | Cat# 2289; RRID: AB_2315049 |
| (D9E) | Technology | |
| Mouse anti-Akt (2H10) | Cell Signaling | Cat# 2967; RRID: AB_331160 |
| | Technology | |
| Rabbit Tuberin/TSC2 | Cell Signaling | Cat# 4308; RRID: AB_10547134 |
| (D93F12) | Technology | |
| XP ® | | |
| Rat anti-Hsp90 (16F1) | Enzo Life Sciences | Cat# ADI-SPA-835; |
| | | RRID: AB_11181205 |
| Anti-mouse secondary | Cell Signaling | Cat# 7076; RRID: AB_330924 |
| | Technology | |
| Anti-rabbit secondary | Cell Signaling | Cat# 7074; RRID: AB_2099233 |
| | Technology | |

Bacterial and Virus Strains

| | | |
|---|---|---|
| BL21(DE3) | EMD Millipore | Cat# 69450 |
| DH5-alpha | Goldbio | Cat# CC-203 |
| Electrocompetent E coli | | |

Biological Samples
Chemicals, Peptides, and Recombinant Proteins

| | | |
|---|---|---|
| IC261 | Abcam | Cat# ab145189 |
| Z-VAD-FMK pan- | Enzo Life Sciences | Cat# ALX-260-020-M001 |
| caspase Inhibitor | | |
| PP5 (PPP5C) Human | OriGene Technologies | Cat# SR321403; SKU# |
| siRNA Oligo Duplex | | SR321403A; SKU# |
| | | SR321403B; SKU# SR321403C |
| Universal scrambled negative | OriGene Technologies | Cat# SR30004 |
| control siRNA duplex | | |
| Compound P0-P13 | This Paper | |
| Biotin-P5 | This Paper | |
| Biotin-P13 | This Paper | |
| BODIPY-P13 | This Paper | |
| P13 derivatives | This Paper | |
| Phos-Ser211-GR peptide | ThermoFisher | This paper |
| ([NH2]PGKETNE[pS]PWRSDL | Scientific custom | |
| L[COOH]) | synthesized | |
| SNX2112 | Duke University; | CAS# 908112-43-6 |
| | Dr. Timothy | |
| | Haystead 61 | |

Critical Commercial Assays

| | | |
|---|---|---|
| Mirus TransIT-2020 | MirusBio | Cat# MIR5405 |
| Anti-FLAG M2 affinity gel | Sigma-Aldrich | Cat# A2220 |
| Protein G agarose | ThermoFisher | Cat# 15-920-010 |
| | Scientific | |
| Pierce Anti-HA Agarose | ThermoFisher | Cat# PI26182 |
| | Scientific | |
| Ni-NTA Agarose | ThermoFisher | Cat# 88221 |
| | Scientific | |
| Quick Cell Proliferation Kit | BioVision | Cat# K302-500; CAS# 150849- |
| Plus | | 52-8 |
| PiPer ™ Phosphate Assay Kit | ThermoFisher | Cat# P22061 |
| | Scientific | |
| ANNEXIN V: FITC assay Kit | BIO-RAD | Cat# ANNEX300F |

Experimental Models: Cell Lines

| | | |
|---|---|---|
| 786-O | ATCC | Cat# CRL-1932 |
| A498 | ATCC | Cat# HTB-44 |
| WT HAP-1 | Horizon Discovery | Cat# C631 |
| PP5 HAP-1 KO | Horizon Discovery | Cat# HZGHC003163c001 |
| FADD HAP-1 KO | Horizon Discovery | Cat# HZGHC002596c006 |
| RIPK1 HAP1-KO | Horizon Discovery | Cat# HZGHC000060c015 |
| HEK293 | ATCC | Cat# CRL-1573 |
| HK-2 | ATCC | Cat# CRL-2190 |
| Caki-1 | ATCC | Cat# HTB-46 |
| Caki-2 | ATCC | Cat# HTB-47 |

Experimental Models: Organisms/Strains
Oligonucleotides

| | | |
|---|---|---|
| DNA primers | Eurofins Genomics | See Supplemental Table S1 |

Recombinant DNA

| | | |
|---|---|---|
| pcDNA3-PP5-FLAG | 2 | n/a |
| pcDNA3-HA-RIPK1 | 38 | Addgene plasmid # |
| | | 78834; |
| | | RRID: Addgene_78834 |
| pcDNA3-FLAG-FADD | 31 | Addgene plasmid # |
| | | 78802; |
| | | RRID: Addgene_78802 |
| pcDNA3-PP5-FLAG-M309C | 5 | n/a |
| pcDNA3-PP5-FLAG-W386F | 5 | n/a |
| pGEX6P1-PP5 | 5 | n/a |

Software and Algorithms

| | |
|---|---|
| Biorender | https://biorender.com/ |
| PyMOL version 2.5.4 for | https://pymol.org/2/ |
| windows | |
| GraphPad Prism version | GraphPad Software, La Jolla, |
| 9.5.0 for windows | California, USA, |
| | www.graphpad.com |
| FlowJo 10.7.1 for windows | https://www.flowjo.com/ |
| Chemdraw 20.1 | https://perkinelmerinformatics.com/ |
| | products/research/chemdraw |

REFERENCES (All references are herein entirely incorporated by reference): Golden, T., Aragon, I. V., Rutland, B., Tucker, J. A., Shevde, L. A., Samant, R. S., Zhou, G., Amable, L., Skarra, D., and Honkanen, R. E. (2008). Elevated levels of Ser/Thr protein phosphatase 5 (PP5) in human breast cancer. Biochim Biophys Acta 1782, 259-270. 10.1016/j.bbadis.2008.01.004. Dushukyan, N., Dunn, D. M., Sager, R. A., Woodford, M. R., Loiselle, D. R., Daneshvar, M., Baker-Williams, A. J., Chisholm, J. D., Truman, A. W., Vaughan, C. K., et al. (2017). Phosphorylation and Ubiquitination Regulate Protein Phosphatase 5 Activity and Its Prosurvival Role in Kidney Cancer. Cell reports 21, 1883-1895. 10.1016/j.celrep.2017.10.074; Sager, R. A., Dushukyan, N., Woodford, M., and Mollapour, M. (2020). Structure and function of the co-chaperone protein phosphatase 5 in cancer. Cell Stress Chaperones 25, 383-394. 10.1007/s12192-020-01091-3; Oberoi, J., Guiu, X. A., Outwin, E. A., Schellenberger, P., Roumeliotis, T. I., Choudhary, J. S., and Pearl, L. H. (2022). HSP90-CDC37-PP5 forms a structural platform for kinase dephosphorylation. Nature communications 13, 7343. 10.1038/s41467-022-35143-2; Jaime-Garza, M., Nowotny, C. A., Coutandin, D., Wang, F., Tabios, M., and Agard, D. A. (2023). Hsp90 provides a platform for kinase dephosphorylation by PP5. Nature communications 14, 2197. 10.1038/s41467-023-37659-7; Oberoi, J., Dunn, D. M., Woodford, M. R., Mariotti, L., Schulman, J., Bourboulia, D., Mollapour, M., and Vaughan, C. K. (2016). Structural and functional basis of protein phosphatase 5 substrate specificity. Proc Natl Acad Sci USA. 10.1073/pnas.1603059113; Shi, Y. (2009). Serine/threonine phosphatases: mechanism through structure. Cell 139, 468-484. 10.1016/j.cell.2009.10.006; Schopf, F. H., Biebl, M. M., and Buchner, J. (2017). The HSP90 chaperone machinery. Nat Rev Mol Cell Biol 18, 345-360. 10.1038/nrm.2017.20; Cox, M. B., and Johnson, J. L. (2011). The Role of p23, Hop, Immunophilins, and Other Co-chaperones in Regulating Hsp90 Function. Methods Mol Biol 787, 45-66. 10.1007/978-1-61779-295-3_4; Cliff, M. J., Harris, R., Barford, D., Ladbury, J. E., and Williams, M. A. (2006). Conformational diversity in the TPR domain-mediated interaction of protein phosphatase 5 with Hsp90. Structure 14, 415-426. S0969-2126(06)00072-4 [pii]10.1016/j.str.2005.12.009; Haslbeck, V., Eckl, J. M., Drazic, A., Rutz, D. A., Lorenz, O. R., Zimmermann, K., Kriehuber, T., Lindemann, C., Madl, T., and Richter, K. (2015). The activity of protein phosphatase 5 towards native clients is modulated by the middle- and C-terminal domains of Hsp90. Scientific reports 5, 17058. 10.1038/srep17058; Kang, H., Sayner, S. L., Gross, K. L., Russell, L. C., and Chinkers, M. (2001). Identification of amino acids in the tetratricopeptide repeat and C-terminal domains of protein phosphatase 5 involved in autoinhibition and lipid activation. Biochemistry 40, 10485-10490; Ramsey, A. J., and Chinkers, M. (2002). Identification of potential physiological activators of protein phosphatase 5. Biochemistry 41, 5625-5632. bi016090h [pii]; Yang, J., Roe, S. M., Cliff, M. J., Williams, M. A., Ladbury, J. E., Cohen, P. T., and Barford, D. (2005). Molecular basis for TPR domain-mediated regulation of protein phosphatase 5. EMBO J 24, 1-10. 7600496 [pii]10.1038/sj.emboj.7600496; Zeke, T., Morrice, N., Vazquez-Martin, C., and Cohen, P. T. (2005). Human protein phosphatase 5 dissociates from heat-shock proteins and is proteolytically activated in response to arachidonic acid and the microtubule-depolymerizing drug nocodazole. Biochem J 385, 45-56. 10.1042/BJ20040690 BJ20040690 [pii]; Vaughan, C. K., Mollapour, M., Smith, J. R., Truman, A., Hu, B., Good, V. M., Panaretou, B., Neckers, L., Clarke, P. A., Workman, P., et al. (2008). Hsp90-dependent activation of protein kinases is regulated by chaperone-targeted dephosphorylation of Cdc37. Mol Cell 31, 886-895. S1097-2765(08)00541-8 [pii]10.1016/j.molcel.2008.07.021; Chatterjee, A., Wang, L., Armstrong, D. L., and Rossie, S. (2010). Activated Rac1 GTPase translocates protein phosphatase 5 to the cell membrane and stimulates phosphatase activity in vitro. J Biol Chem 285, 3872-3882. 10.1074/jbc.M109.088427; Clifford, S. C., Astuti, D., Hooper, L., Maxwell, P. H., Ratcliffe, P. J., and Maher, E. R. (2001). The pVHL-associated SCF ubiquitin ligase complex: molecular genetic analysis of elongin B and C, Rbx1 and HIF-1alpha in renal cell carcinoma. Oncogene 20, 5067-5074. 10.1038/sj.onc.1204602; Maxwell, P. H., Wiesener, M. S., Chang, G. W., Clifford, S. C., Vaux, E. C., Cockman, M. E., Wykoff, C. C., Pugh, C. W., Maher, E. R., and Ratcliffe, P. J. (1999). The tumour suppressor protein VHL targets hypoxia-inducible factors for oxygen-dependent proteolysis. Nature 399, 271-275. 10.1038/20459; Kondo, K., Klco, J., Nakamura, E., Lechpammer, M., and Kaelin, W. G., Jr. (2002). Inhibition of HIF is necessary for tumor suppression by the von Hippel-Lindau protein. Cancer Cell 1, 237-246; Woodford, M. R., Backe, S. J., Wengert, L. A., Dunn, D. M., Bourboulia, D., and Mollapour, M. (2021). Hsp90 chaperone code and the tumor suppressor VHL cooperatively regulate the mitotic checkpoint. Cell Stress Chaperones. 10.1007/s12192-021-01240-2; Hasanov, E., Chen, G., Chowdhury, P., Weldon, J., Ding, Z., Jonasch, E., Sen, S., Walker, C. L., and Dere, R. (2017). Ubiquitination and regulation of AURKA identifies a hypoxia-independent E3 ligase activity of VHL. Oncogene. 10.1038/onc.2016.495; Bluyssen, H. A., Lolkema, M. P., van Beest, M., Boone, M., Snijckers, C. M., Los, M., Gebbink, M. F., Braam, B., Holstege, F. C., Giles, R. H., and Voest, E. E. (2004). Fibronectin is a hypoxia-independent target of the tumor suppressor VHL. FEBS Lett 556, 137-142. 10.1016/s0014-5793(03)01392-9; Linehan, W. M., Schmidt, L. S., Crooks, D. R., Wei, D., Srinivasan, R., Lang, M., and Ricketts, C. J. (2019). The Metabolic Basis of Kidney Cancer. Cancer Discov. 10.1158/2159-8290.CD-18-1354; Choueiri, T. K., and Kaelin, W. G., Jr. (2020). Targeting the HIF2-VEGF axis in renal cell carcinoma. Nat Med 26, 1519-1530. 10.1038/s41591-020-1093-z; Iliopoulos, O., Kibel, A., Gray, S., and Kaelin, W. G., Jr. (1995). Tumour suppression by the human von Hippel-Lindau gene product. Nat Med 1, 822-826; Bertini, I., Calderone, V., Fragai, M., Luchinat, C., and Talluri, E. (2009). Structural basis of serine/threonine phosphatase inhibition by the archetypal small molecules cantharidin and norcantharidin. J Med Chem 52, 4838-4843. 10.1021/jm900610k; Irwin, J. J. (2008). Using ZINC to acquire a virtual screening library. Curr Protoc Bioinformatics Chapter 14, Unit 14 16. 10.1002/0471250953.bi1406s22; Irwin, J. J., Raushel, F. M., and Shoichet, B. K. (2005). Virtual screening against metalloenzymes for inhibitors and substrates. Biochemistry 44, 12316-12328. 10.1021/bi050801k; Irwin, J. J., Shoichet, B. K., Mysinger, M. M., Huang, N., Colizzi, F., Wassam, P., and Cao, Y. (2009). Automated docking screens: a feasibility study. J Med Chem 52, 5712-5720. 10.1021/jm9006966; Alappat, E. C., Feig, C., Boyerinas, B., Volkland, J., Samuels, M., Murmann, A. E., Thorburn, A., Kidd, V. J., Slaughter, C. A., Osborn, S. L., et al. (2005). Phosphorylation of FADD at serine 194 by CKlalpha regulates its nonapoptotic activities. Mol Cell 19, 321-332. 10.1016/j.molcel.2005.06.024; Lee, E. W., Kim, J. H., Ahn, Y. H., Seo, J., Ko, A., Jeong, M., Kim, S. J., Ro, J. Y., Park, K. M., Lee, H. W., et al. (2012). Ubiquitination and degradation of the FADD adaptor protein regulate death receptor-mediated apoptosis and necroptosis. Nature communications 3, 978. 10.1038/ncomms1981; Lee, E. W., Seo, J., Jeong, M., Lee, S., and Song, J. (2012). The roles of FADD in extrinsic apoptosis and necroptosis. BMB Rep 45, 496-508. 10.5483/bmbrep.2012.45.9.186; Lee, H. J., Pyo, J. O., Oh, Y., Kim, H. J., Hong, S. H., Jeon, Y. J., Kim, H., Cho, D. H., Woo, H. N., Song, S., et al. (2007). AK2 activates a novel apoptotic pathway through formation of a complex with FADD and caspase-10. Nature cell biology 9, 1303-1310. 10.1038/ncb1650; Shimada, K., Matsuyoshi, S., Nakamura, M., Ishida, E., Kishi, M., and Konishi, N. (2004). Phosphorylation of FADD is critical for sensitivity to anticancer drug-induced apoptosis. Carcinogenesis 25, 1089-1097. 10.1093/carcin/bgh130; Kotecki, M., Reddy, P. S., and Cochran, B. H. (1999). Isolation and characterization of a near-haploid human cell line. Exp Cell Res 252, 273-280. 10.1006/excr.1999.4656; Meng, H., Liu, Z., Li, X., Wang, H., Jin, T., Wu, G., Shan, B., Christofferson, D. E., Qi, C., Yu, Q., et al. (2018). Death-domain dimerization-mediated activation of RIPK1 controls necroptosis and RIPK1-dependent apoptosis. Proc Natl Acad Sci USA 115, E2001-E2009. 10.1073/pnas.1722013115; Vanden Berghe, T., van Loo, G., Saelens, X., Van Gurp, M., Brouckaert, G., Kalai, M., Declercq, W., and Vandenabeele, P. (2004). Differential signaling to apoptotic and necrotic cell death by Fas-associated death domain protein FADD. J Biol Chem 279, 7925-7933. 10.1074/jbc.M307807200 M307807200 [pii]; Seo, J., Lee, E. W., Sung, H., Seong, D., Dondelinger, Y., Shin, J., Jeong, M., Lee, H. K., Kim, J. H., Han, S. Y., et al. (2016). CHIP controls necroptosis through ubiquitylation- and lysosome-dependent degradation of RIPK3. Nature cell biology 18, 291-302. 10.1038/ncb3314; Woodford, M. R., Sager, R. A., Marris, E., Dunn, D. M., Blanden, A. R., Murphy, R. L., Rensing, N., Shapiro, O., Panaretou, B., Prodromou, C., et al. (2017). Tumor suppressor Tsc1 is a new Hsp90 co-chaperone that facilitates folding of kinase and non-kinase clients. EMBO J. 10.15252/embj.201796700; Barrott, J. J., Hughes, P. F., Osada, T., Yang, X. Y., Hartman, Z. C., Loiselle, D. R., Spector, N. L., Neckers, L., Rajaram, N., Hu, F., et al. (2013). Optical and radioiodinated tethered Hsp90 inhibitors reveal selective internalization of ectopic Hsp90 in malignant breast tumor cells. Chem Biol 20, 1187-1197. 10.1016/j.chembiol.2013.08.004; Yang, C. K., and He, S. D. (2016). Heat shock protein 90 regulates necroptosis by modulating multiple signaling effectors. Cell Death Dis 7, e2126. 10.1038/cddis.2016.25; D'Arcy, B. M., Prakash, A., and Honkanen, R. E. (2019). Targeting phosphatases in cancer: suppression of many versus the ablation of one. Oncotarget 10, 6543-6545. 10.18632/oncotarget.27201; Jang, M. S., Lee, S. J., Kim, C. J., Lee, C. W., and Kim, E. (2011). Phosphorylation by polo-like kinase 1 induces the tumor-suppressing activity of FADD. Oncogene 30, 471-481. 10.1038/onc.2010.423; Xu, H., He, L., Feng, X., Kapoor, A., and Tang, D. (2009). Specific reduction of fas-associated protein with death domain (FADD) in clear cell renal cell carcinoma. Cancer Invest 27, 836-843. 10.1080/07357900902849681; Seyrek, K., Ivanisenko, N. V., Richter, M., Hillert, L. K., Konig, C., and Lavrik, I. N. (2020). Controlling Cell Death through Post-translational Modifications of DED Proteins. Trends Cell Biol 30, 354-369. 10.1016/j.tcb.2020.02.006; Vilmont, V., Filhol, O., Hesse, A. M., Coute, Y., Hue, C., Remy-Tourneur, L., Mistou, S., Cochet, C., and Chiocchia, G. (2015). Modulatory role of the anti-apoptotic protein kinase CK2 in the sub-cellular localization of Fas associated death domain protein (FADD). Biochim Biophys Acta 1853, 2885-2896. 10.1016/j.bbamcr.2015.08.001; Swingle, M. R., and Honkanen, R. E. (2014). Development and validation of a robust and sensitive assay for the discovery of selective inhibitors for serine/threonine protein phosphatases PP1alpha (PPP1C) and PP5 (PPP5C). Assay and drug development technologies 12, 481-496. 10.1089/adt.2014.603; Liu, J. Y., Chen, X. E., and Zhang, Y. L. (2015). Insights into the key interactions between human protein phosphatase 5 and cantharidin using molecular dynamics and site-directed mutagenesis bioassays. Scientific reports 5, 12359. 10.1038/srep12359; Swingle, M., Volmar, C. H., Saldanha, S. A., Chase, P., Eberhart, C., Salter, E. A., D'Arcy, B., Schroeder, C. E., Golden, J. E., Wierzbicki, A., et al. (2017). An Ultra-High-Throughput Screen for Catalytic Inhibitors of Serine/Threonine Protein Phosphatases: Types 1 and 5 (PP1C and PP5C). SLAS Discov 22, 21-31. 10.1177/1087057116668852; Zhang, Q., Wu, X., Zhang, H., Wu, Q., Fu, M., Hua, L., Zhu, X., Guo, Y., Zhang, L., You, Q., and Wang, L. (2023). Protein Phosphatase 5-Recruiting Chimeras for Accelerating Apoptosis-Signal-Regulated Kinase 1 Dephosphorylation with Antiproliferative Activity. J Am Chem Soc 145, 1118-1128. 10.1021/jacs.2c10759; Hong, C. S., Ho, W., Zhang, C., Yang, C., Elder, J. B., and Zhuang, Z. (2015). LB100, a small molecule inhibitor of PP2A with potent chemo- and radio-sensitizing potential. Cancer Biol Ther 16, 821-833. 10.1080/15384047.2015.1040961; D'Arcy, B. M., Swingle, M. R., Papke, C. M., Abney, K. A., Bouska, E. S., Prakash, A., and Honkanen, R. E. (2019). The Antitumor Drug LB-100 Is a Catalytic Inhibitor of Protein Phosphatase 2A (PPP2CA) and 5 (PPP5C) Coordinating with the Active-Site Catalytic Metals in PPP5C. Mol Cancer Ther 18, 556-566. 10.1158/1535-7163.MCT-17-1143; Shuhaibar, L. C., Kaci, N., Egbert, J. R., Horville, T., Loisay, L., Vigone, G., Uliasz, T. F., Dambroise, E., Swingle, M. R., Honkanen, R. E., et al. (2021). Phosphatase inhibition by LB-100 enhances BMN-111 stimulation of bone growth. JCI Insight 6. 10.1172/jci.insight.141426; Mollapour, M., Tsutsumi, S., Donnelly, A. C., Beebe, K., Tokita, M. J., Lee, M. J., Lee, S., Morra, G., Bourboulia, D., Scroggins, B. T., et al. (2010). Swe1Wee1-dependent tyrosine phosphorylation of Hsp90 regulates distinct facets of chaperone function. Mol Cell 37, 333-343. S1097-2765(10)00030-4 [pii]10.1016/j.molcel.2010.01.005; Woodford, M. R., Truman, A. W., Dunn, D. M., Jensen, S. M., Cotran, R., Bullard, R., Abouelleil, M., Beebe, K., Wolfgeher, D., Wierzbicki, S., et al. (2016). Mps1 Mediated Phosphorylation of Hsp90 Confers Renal Cell Carcinoma Sensitivity and Selectivity to Hsp90 Inhibitors. Cell reports 14, 872-884. 10.1016/j.celrep.2015.12.084; Trott, O., and Olson, A. J. (2010). AutoDock Vina: improving the speed and accuracy of docking with a new scoring function, efficient optimization, and multithreading. J Comput Chem 31, 455-461. 10.1002/jcc.21334; Krajcovicova, S., Stankova, J., Dzubak, P., Hajduch, M., Soural, M., and Urban, M. (2018). A Synthetic Approach for the Rapid Preparation of BODIPY Conjugates and their use in Imaging of Cellular Drug Uptake and Distribution. Chemistry 24, 4957-4966. 10.1002/chem.201706093; White, J., and Whiteley, C. G. (1993). Use of Organoboranes in the Synthesis of Pheromones: A Convenient Synthesis of (Z)-6-Heneicosen-11-one and (Z)-5-Undecen-2-one, Pheromones from the Douglas Fir Tussock Moth and the Bontebok, Respectively. Synthesis 1993, 1141-1144. 10.1055/s-1993-26016; Cer, R. Z., Mudunuri, U., Stephens, R., and Lebeda, F. J. (2009). IC50-to-Ki: a web-based tool for converting IC50 to Ki values for inhibitors of enzyme activity and ligand binding. Nucleic Acids Res 37, W441-445. 10.1093/nar/gkp253.

The entire disclosure of all applications, patents, and publications cited herein are herein incorporated by reference in their entirety. While the foregoing is directed to embodiments of the present disclosure, other and further embodiments of the disclosure may be devised without departing from the basic scope thereof.

What is claimed is:

1. A method of treating cancer, comprising: a) providing i) a subject with cancer, ii) one or more protein phosphatase-5 inhibitors, and b) treating said subject with the one or more PP5 inhibitors.

2. The method of claim 1, wherein treating comprises administering a therapeutically effective amount of one or more protein phosphatase-5 inhibitors to the subject.

3. The method of claim 1, wherein the cancer is renal cell carcinoma.

4. The method of claim 3, wherein the renal cancer is clear cell renal cell carcinoma.

5. The method of claim 1, wherein the one or more protein phosphatase-5 inhibitors are characterized as a PP5 specific inhibitor.

6. The method of claim 1, wherein the one or more protein phosphatase-5 inhibitors are selected from the group consisting of: 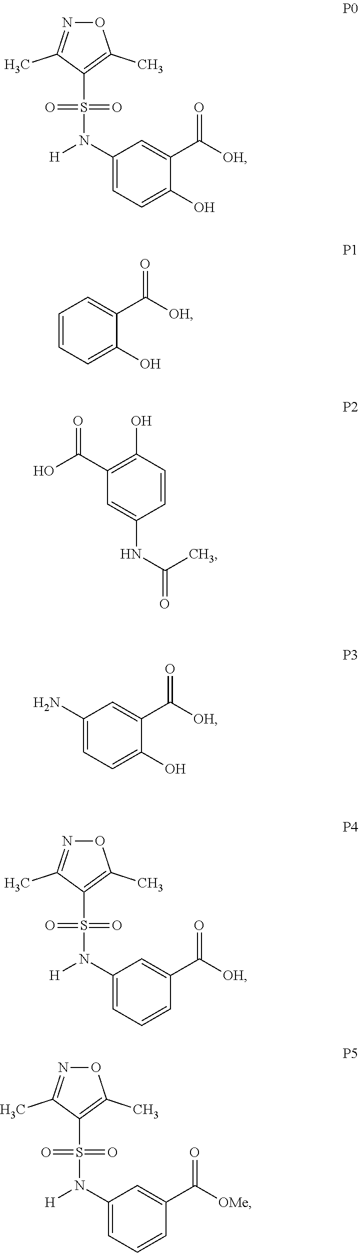 [SMILES: Cc1noc(C)c1S(=O)(=O)Nc1ccc(O)c(C(=O)O)c1 ; O=C(O)c1ccccc1O ; CC(=O)Nc1ccc(O)c(C(=O)O)c1 ; Nc1ccc(O)c(C(=O)O)c1 ; Cc1noc(C)c1S(=O)(=O)Nc1cccc(C(=O)O)c1 ; COC(=O)c1cccc(NS(=O)(=O)c2c(C)noc2C)c1] 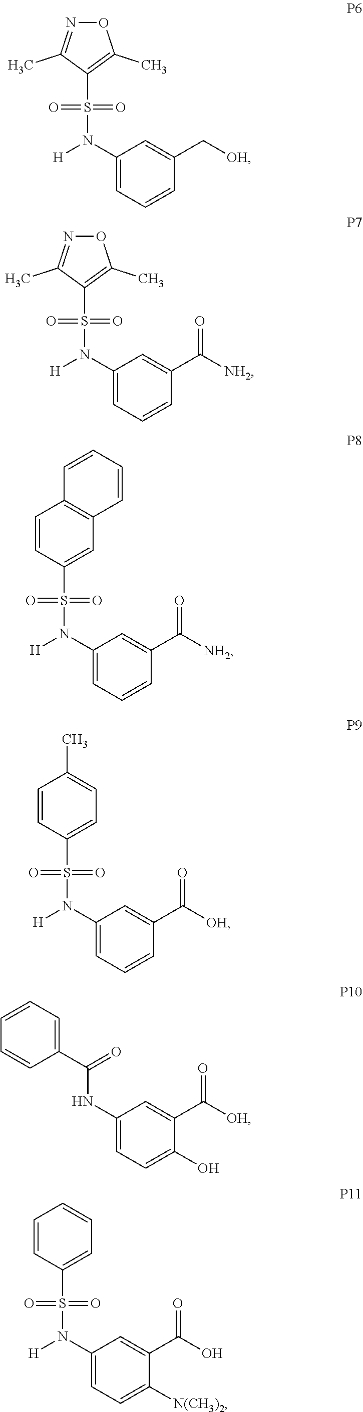 [SMILES: Cc1noc(C)c1S(=O)(=O)Nc1cccc(CO)c1 ; Cc1noc(C)c1S(=O)(=O)Nc1cccc(C(N)=O)c1 ; NC(=O)c1cccc(NS(=O)(=O)c2ccc3ccccc3c2)c1 ; Cc1ccc(S(=O)(=O)Nc2cccc(C(=O)O)c2)cc1 ; O=C(Nc1ccc(O)c(C(=O)O)c1)c1ccccc1 ; CN(C)c1ccc(NS(=O)(=O)c2ccccc2)cc1C(=O)O] 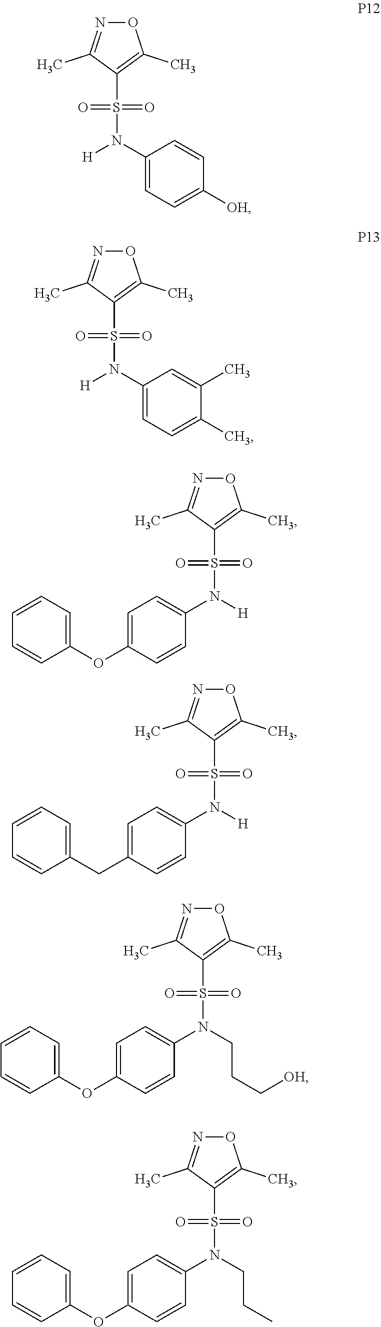 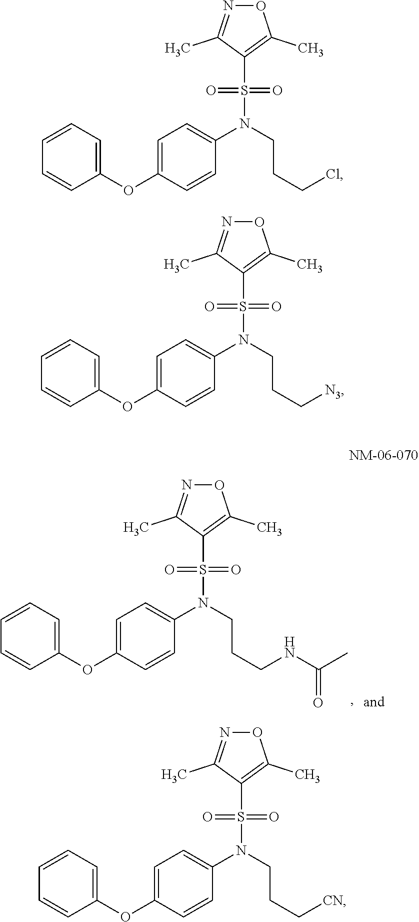 [SMILES: Cc1noc(C)c1S(=O)(=O)N(CCCCl)c1ccc(Oc2ccccc2)cc1 ; Cc1noc(C)c1S(=O)(=O)N(CCCN=[N+]=[N-])c1ccc(Oc2ccccc2)cc1 ; CC(=O)NCCCN(c1ccc(Oc2ccccc2)cc1)S(=O)(=O)c1c(C)noc1C ; Cc1noc(C)c1S(=O)(=O)N(CCCC#N)c1ccc(Oc2ccccc2)cc1]
or a pharmaceutically acceptable salt thereof, and combinations thereof.

7. The method of claim 1, wherein treating the subject with the one or more protein phosphatase-5 inhibitors is sequential.

8. The method of claim 1, wherein treating the subject with the one or more protein phosphatase-5 inhibitors is simultaneous.

9. The method of claim 1, wherein treating with the one or more protein phosphatase-5 inhibitors results in reduced proliferation of at least some of the cancer cells within said subject.

10. A method of treating cancer, comprising: a) providing i) a subject with cancer and one or more compounds of: 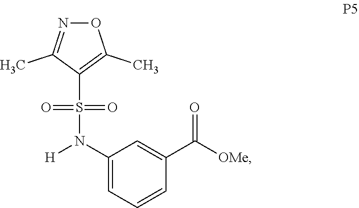 [SMILES: COC(=O)c1cccc(NS(=O)(=O)c2c(C)noc2C)c1]
or a pharmaceutically acceptable salt thereof, 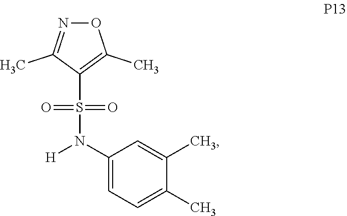
or a pharmaceutically acceptable salt thereof,
and b) treating the subject with the one or more compounds or pharmaceutically acceptable salts thereof.

11. A method of treating cancer, comprising: a) providing i) a subject with cancer and one or more compounds of: 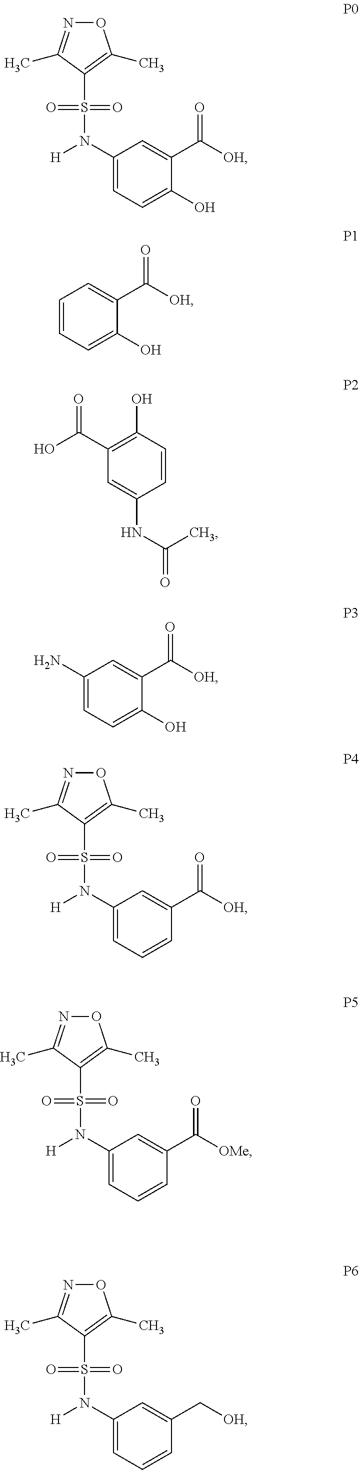 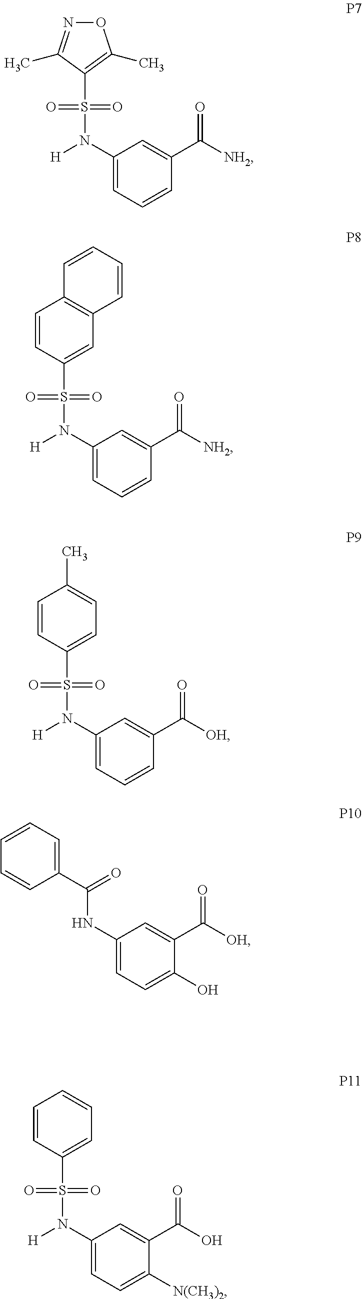 [SMILES: Cc1noc(C)c1S(=O)(=O)Nc1cccc(C(N)=O)c1 ; NC(=O)c1cccc(NS(=O)(=O)c2ccc3ccccc3c2)c1 ; Cc1ccc(S(=O)(=O)Nc2cccc(C(=O)O)c2)cc1 ; O=C(Nc1ccc(O)c(C(=O)O)c1)c1ccccc1 ; CN(C)c1ccc(NS(=O)(=O)c2ccccc2)cc1C(=O)O] 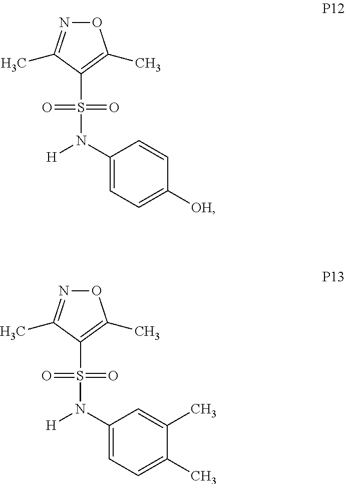 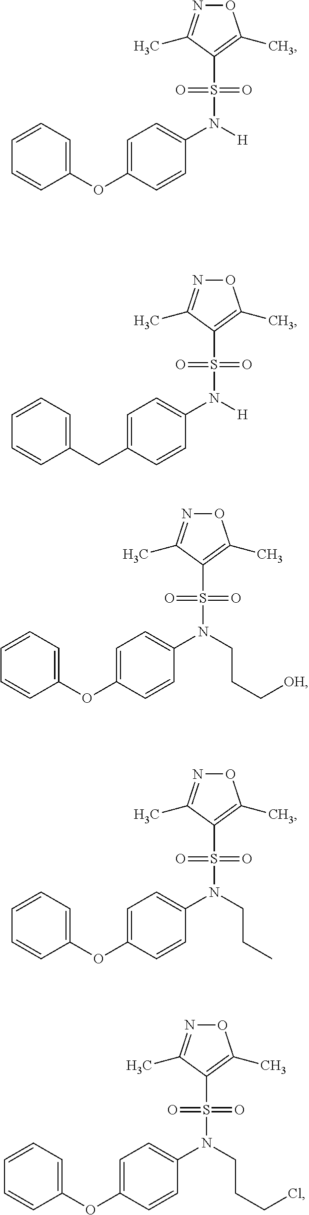 [SMILES: Cc1noc(C)c1S(=O)(=O)Nc1ccc(Oc2ccccc2)cc1 ; Cc1noc(C)c1S(=O)(=O)Nc1ccc(Cc2ccccc2)cc1 ; Cc1noc(C)c1S(=O)(=O)N(CCCO)c1ccc(Oc2ccccc2)cc1 ; CCCN(c1ccc(Oc2ccccc2)cc1)S(=O)(=O)c1c(C)noc1C ; Cc1noc(C)c1S(=O)(=O)N(CCCCl)c1ccc(Oc2ccccc2)cc1] 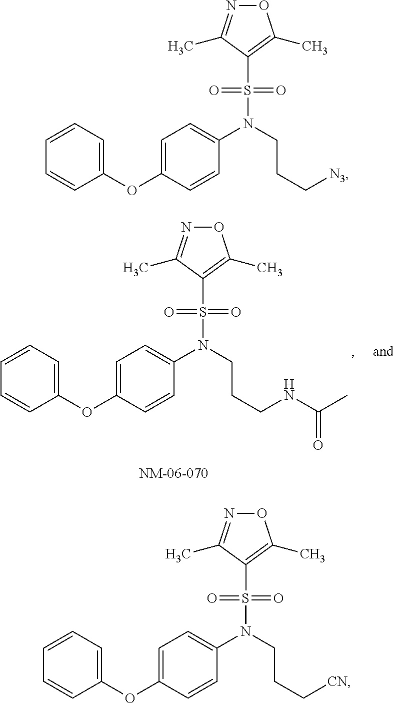
and b) treating the subject with the one or more compounds or pharmaceutically acceptable salts thereof.

11. A pharmaceutical anticancer composition comprising one or more PP5 inhibitors, or pharmaceutically acceptable salts thereof.

12. The pharmaceutical anticancer composition of claim 11, wherein the composition is characterized as PP5 specific.

13. The pharmaceutical anticancer composition of claim 11, wherein the one or more PP5 inhibitors are selected from the group consisting of: 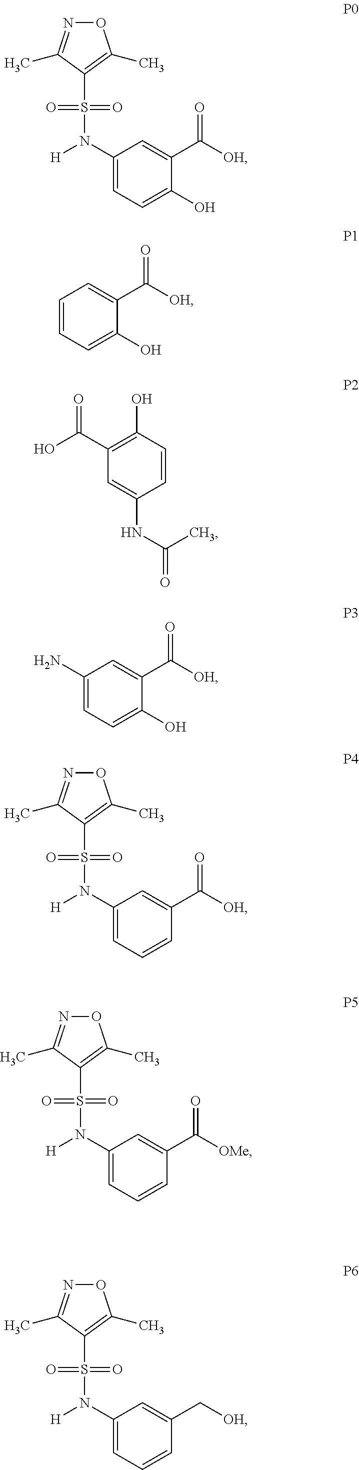 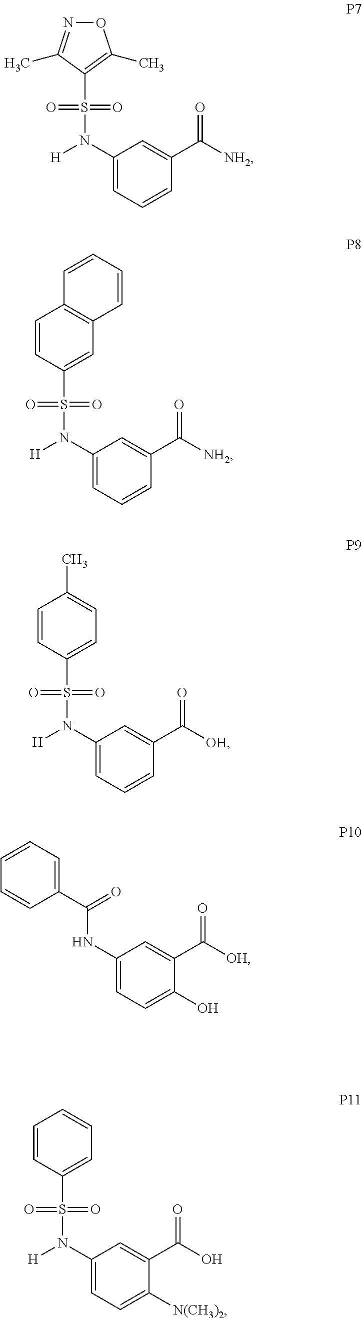 [SMILES: Cc1noc(C)c1S(=O)(=O)Nc1cccc(C(N)=O)c1 ; NC(=O)c1cccc(NS(=O)(=O)c2ccc3ccccc3c2)c1 ; Cc1ccc(S(=O)(=O)Nc2cccc(C(=O)O)c2)cc1 ; O=C(Nc1ccc(O)c(C(=O)O)c1)c1ccccc1 ; CN(C)c1ccc(NS(=O)(=O)c2ccccc2)cc1C(=O)O] 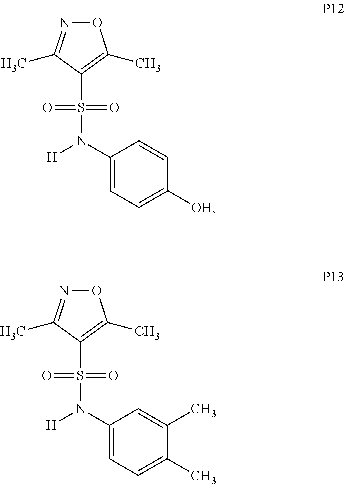 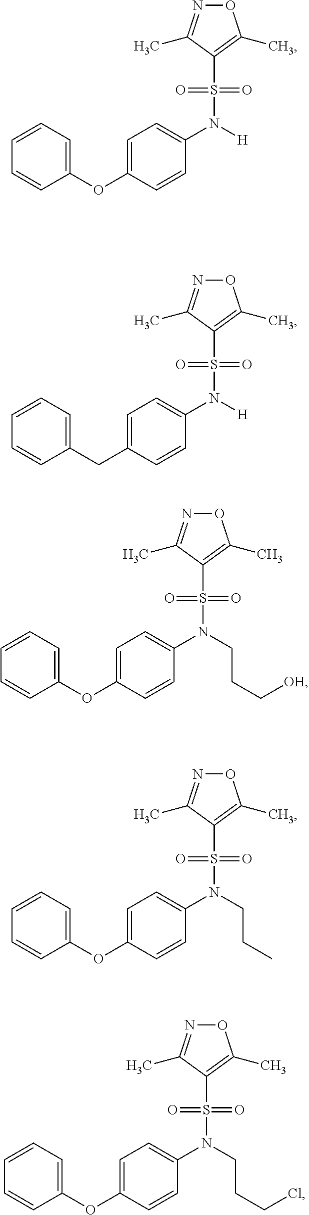 [SMILES: Cc1noc(C)c1S(=O)(=O)Nc1ccc(Oc2ccccc2)cc1 ; Cc1noc(C)c1S(=O)(=O)Nc1ccc(Cc2ccccc2)cc1 ; Cc1noc(C)c1S(=O)(=O)N(CCCO)c1ccc(Oc2ccccc2)cc1 ; CCCN(c1ccc(Oc2ccccc2)cc1)S(=O)(=O)c1c(C)noc1C ; Cc1noc(C)c1S(=O)(=O)N(CCCCl)c1ccc(Oc2ccccc2)cc1] 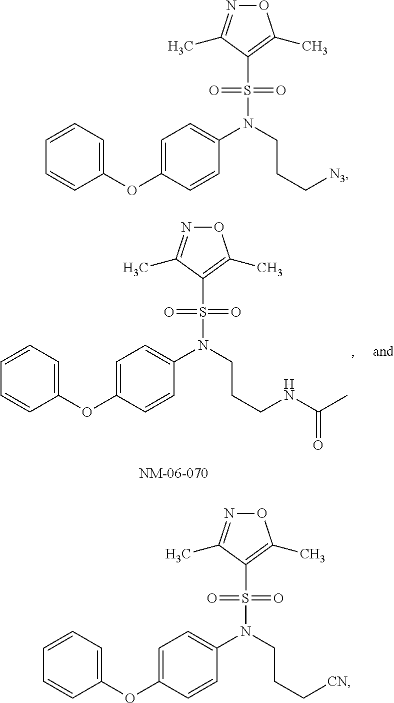
or a pharmaceutically acceptable salt thereof, and combinations thereof.

14. A pharmaceutical anticancer composition comprising one or more PP5 inhibitors selected from the group consisting of: 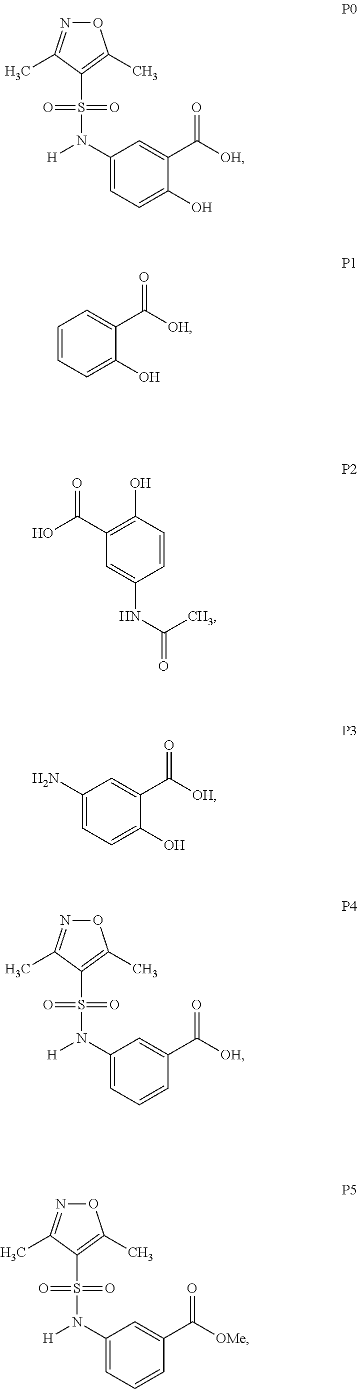 [SMILES: Cc1noc(C)c1S(=O)(=O)Nc1ccc(O)c(C(=O)O)c1 ; O=C(O)c1ccccc1O ; CC(=O)Nc1ccc(O)c(C(=O)O)c1 ; Nc1ccc(O)c(C(=O)O)c1 ; Cc1noc(C)c1S(=O)(=O)Nc1cccc(C(=O)O)c1 ; COC(=O)c1cccc(NS(=O)(=O)c2c(C)noc2C)c1] 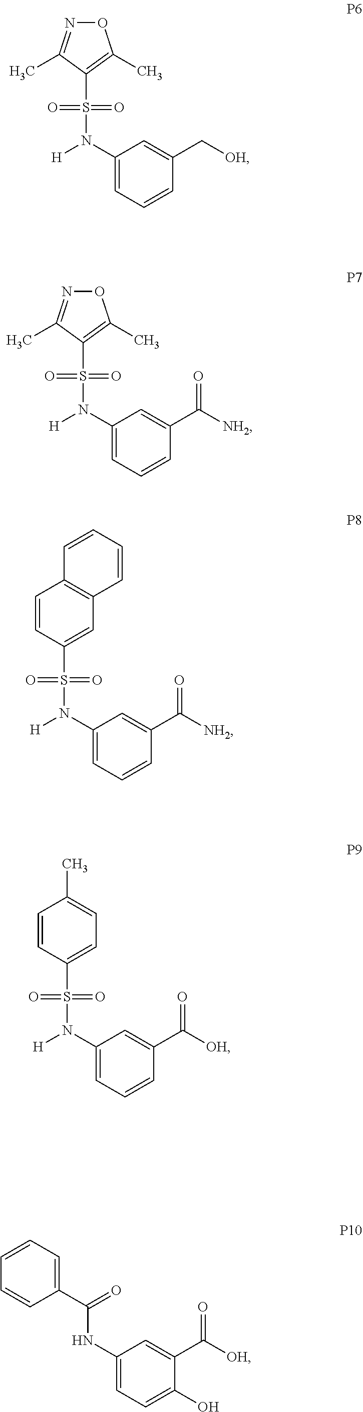 [SMILES: Cc1noc(C)c1S(=O)(=O)Nc1cccc(CO)c1 ; Cc1noc(C)c1S(=O)(=O)Nc1cccc(C(N)=O)c1 ; NC(=O)c1cccc(NS(=O)(=O)c2ccc3ccccc3c2)c1 ; Cc1ccc(S(=O)(=O)Nc2cccc(C(=O)O)c2)cc1 ; O=C(Nc1ccc(O)c(C(=O)O)c1)c1ccccc1] 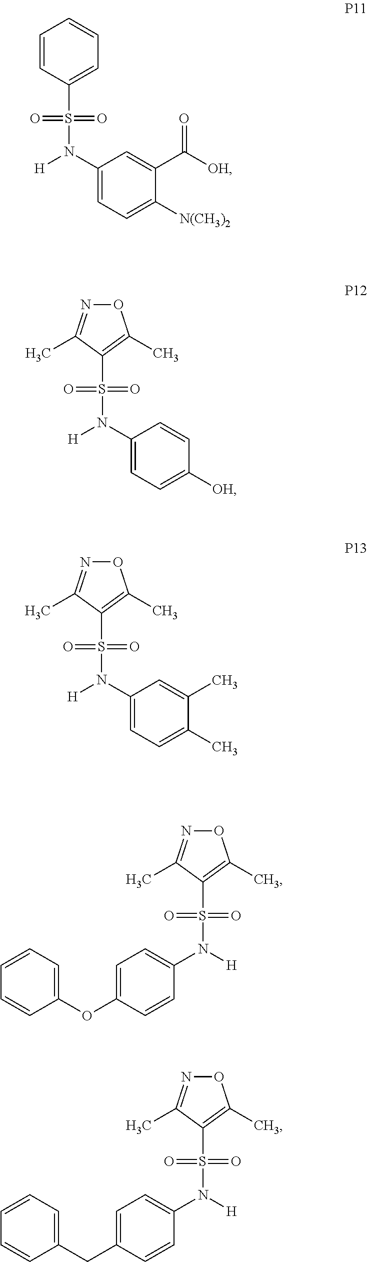 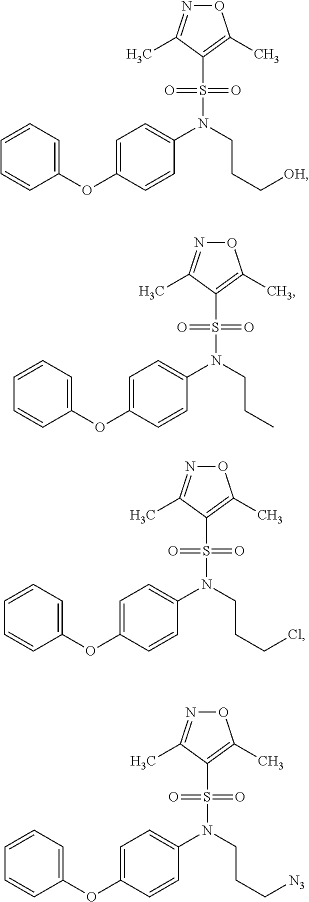 [SMILES: Cc1noc(C)c1S(=O)(=O)N(CCCO)c1ccc(Oc2ccccc2)cc1 ; CCCN(c1ccc(Oc2ccccc2)cc1)S(=O)(=O)c1c(C)noc1C ; Cc1noc(C)c1S(=O)(=O)N(CCCCl)c1ccc(Oc2ccccc2)cc1 ; Cc1noc(C)c1S(=O)(=O)N(CCCN=[N+]=[N-])c1ccc(Oc2ccccc2)cc1] 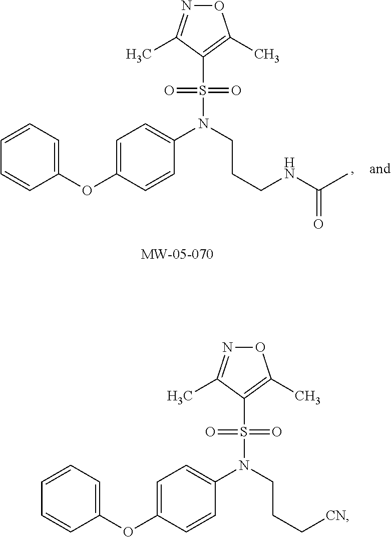 [SMILES: CC(=O)NCCCN(c1ccc(Oc2ccccc2)cc1)S(=O)(=O)c1c(C)noc1C ; Cc1noc(C)c1S(=O)(=O)N(CCCC#N)c1ccc(Oc2ccccc2)cc1]
and combinations thereof.
